(19) United States
(12) Patent Application Publication
(10) Pub. No.: US 2011/0150885 A1
(43) Pub. Date: Jun. 23, 2011

(54) AMINOACYL TRNA SYNTHETASES FOR MODULATING HEMATOPOIESIS

(75) Inventors: Jeffry Dean Watkins, Encinitas, CA (US); Alain P. Vasserot, Carlsbad, CA (US); Rajesh Belani, San Diego, CA (US); Leslie Ann Greene, San Diego, CA (US); Ryan Andrew Adams, San Diego, CA (US)

(73) Assignee: ATYR PHARMA, INC., San Diego, CA (US)

(21) Appl. No.: 12/965,655

(22) Filed: Dec. 10, 2010

Related U.S. Application Data
(60) Provisional application No. 61/285,907, filed on Dec. 11, 2009.
(60) Provisional application No. 61/304,283, filed on Feb. 12, 2010.
(60) Provisional application No. 61/334,127, filed on May 12, 2010.
(60) Provisional application No. 61/359,767, filed on Jun. 29, 2010.

(51) Int. Cl. A61K 39/395; A61K 38/53; C12N 5/078; A61K 35/12; C07H 21/00; C12N 15/63; C12N 5/10; C12N 9/00; C07K 16/18; C07K 14/00; A61P 7/00

(52) U.S. Cl. 424/139.1; 424/94.5; 435/377; 424/93.7; 536/23.2; 435/320.1; 435/325; 435/183; 530/387.9; 530/300

(57) ABSTRACT

Hematopoietic-modulating compositions are provided comprising aminoacyl-tRNA synthetase polypeptides, including active fragments and/or variants thereof, as well as compositions comprising related agents such as antibodies and other binding agents. Also provided are methods of using such compositions in the treatment of conditions that benefit from the modulation of hematopoiesis.

### CROSS REFERENCE TO RELATED APPLICATIONS

This application claims benefit under 35 U.S.C. §119(e) to U.S. Provisional Application No. 61/285,907, filed Dec. 11, 2009; U.S. Provisional Application No. 61/304,283, filed Feb. 12, 2010; U.S. Provisional Application No. 61/334,127, filed May 12, 2010; and U.S. Provisional Application No. 61/359,767, filed Jun. 29, 2010, which are incorporated by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 120161_{—}419_SEQUENCE_LISTING.txt. The text file is 102 KB, was created on Dec. 10, 2010, and is being submitted electronically via EFS-Web.

### BACKGROUND

1. Technical Field

The present invention relates generally to compositions comprising hematopoiesis-modulating aminoacyl-tRNA synthetase (AARS) polypeptides, related agents such as antibodies and binding agents that modulate the activity of the AARS polypeptides, and methods of using such compositions and agents for modulating hematopoiesis.

2. Description of the Related Art

Aminoacyl-tRNA synthetases, which catalyze the aminoacylation of tRNA molecules, are essential for decoding genetic information during the process of translation. In higher eukaryotes, some aminoacyl-tRNA synthetases associate with other polypeptides to form supramolecular multienzyme complexes. Each of the eukaryotic tRNA synthetases consists of a core enzyme, which is closely related to the prokaryotic counterpart of the tRNA synthetase, and one or more additional domains that are appended to the amino-terminal or carboxyl-terminal end of the core enzyme. Human tyrosyl-tRNA synthetase (YRS), for example, has a carboxyl-terminal domain that is not part of prokaryotic and lower eukaryotic YRS molecules.

Aminoacyl tRNA synthetases, such as tyrosyl-tRNA synthetase, tryptophan-tRNA synthetase, and others, are associated with expanded functions in mammalian cells, including activities in signal transduction pathways, among others.

### BRIEF SUMMARY

The present invention stems from the unexpected finding that compositions comprising aminoacyl-tRNA synthetase (AARS) polypeptides, including truncated fragments, splice variants, proteolytic fragments, and variants thereof, are capable of modulating hematopoiesis. In certain instances, these polypeptides reduce erythropoiesis, or the formation of progenitor cells of the erythroid lineage. In certain instances, these polypeptides enhance megakaryopoiesis, thrombopoiesis and/or the formation of megakaryocyte progenitor cells, either in vivo or ex vivo. Other aspects are detailed herein, including the use of agonists and antagonists of these hematopoiesis-modulating AARS polypeptides.

Accordingly, embodiments of the present invention relate to compositions for modulating hematopoiesis, comprising one or more isolated aminoacyl-tRNA synthetase (AARS) polypeptides, or biologically active fragments or variants thereof, wherein the polypeptides modulate hematopoiesis. In certain embodiments, the AARS polypeptide is a tyrosyl-tRNA synthetase (YRS), a tryptophanyl-tRNA synthetase (WRS), a glutaminyl-tRNA synthetase (QRS), a glycyl-tRNA synthetase (GlyRS), a histidyl-tRNA synthetase (His RS), a seryl-tRNA synthetase, a phenylalanyl-tRNA synthetase, an alanyl-tRNA synthetase, an asparaginyl-tRNA synthetase (AsnRS), an aspartyl-tRNA synthetase (AspRS), a cysteinyl-tRNA synthetase (CysRS), a glutamyl-tRNA synthetase, a prolyl-tRNA synthetase (ProRS), an arginyl-tRNA synthetase, an isoleucyl-tRNA synthetase, a leucyl-tRNA synthetase, a lysyl-tRNA synthetase, a threonyl-tRNA synthetase, a methionyl-tRNA synthetases, or a valyl-tRNA synthetase.

Certain embodiments include a proteolytic fragment of the AARS polypeptide. In certain embodiments, the sequence of the proteolytic fragment is derived by incubating the polypeptide with a protease in vitro. In certain embodiments, the sequence of the proteolytic fragment is derived by recombinantly expressing the AARS polypeptide in a cell, wherein the cell comprises one or more recombinant or endogenous proteases. In certain embodiments, the proteolytic fragment comprises the sequence of an endogenous, naturally-occurring human or mouse AARS proteolytic fragment.

In certain embodiments, the aminoacyl-tRNA synthetase is a YRS polypeptide. In certain embodiments, the YRS polypeptide is truncated at its C-terminus. In certain embodiments, the YRS polypeptide comprises the amino acid sequence of SEQ ID NO: 1, 2, 3, 6, 8, 10, 12, or 14, wherein at least about 1-50, 50-100, 100-150, 150-200, or about 200-250 amino acid residues are truncated from its C-terminus.

In certain embodiments, the YRS polypeptide is truncated at its N-terminus. In certain embodiments, the YRS polypeptide comprises the amino acid sequence of SEQ ID NO: 1, 2, 3, 6, 8, 10, 12, or 14, wherein at least about 1-50, 50-100, 50-100, 100-150, 150-200, or about 200-250 amino acid residues are truncated from its N-terminus.

In certain embodiments, the YRS polypeptide comprises an amino acid sequence at least 80%, 90%, 95%, 98%, or 100% identical to the amino acid sequence set forth in SEQ ID NO:2, wherein the alanine at position 341 is not substituted with a tyrosine. In certain embodiments, the YRS polypeptide comprises an amino acid sequence at least 80%, 90%, 95%, 98%, or 100% identical to the amino acid sequence set forth in SEQ ID NO: 1, 2, 3, 6, 8, 10, 12, or 14.

In certain embodiments, the aminoacyl-tRNA synthetase is a GlyRS polypeptide. In certain embodiments, the GlyRS polypeptide is a fragment of the full length human glycyl-tRNA synthetase sequence set forth in SEQ ID NO:16. In certain embodiments, the fragment comprises amino acid residues 367-438 of SEQ ID NO:16, or an active variant thereof. In certain embodiments, the GlyRS polypeptide comprises an amino acid sequence at least 80%, 90%, 95%, 98%, or 100% identical to the amino acid sequence set forth in SEQ ID NO:16. In certain embodiments, the GlyRS polypeptide comprises amino acid residues 57-685, 214-685, 239-685, 311-685, 439-685, 511-658, 214-438, 367-438, 214-420, 214-338, 85-127 1-213, 1-61, 85-214, 333-685, 128-685, 265-685, 483-685 or 25-56 of SEQ ID NO:16, or an active fragment thereof.

In certain embodiments, the aminoacyl-tRNA synthetase is a QRS polypeptide. In certain embodiments, the QRS polypeptide comprises an amino acid sequence at least 80%, 90%, 95%, 98%, or 100% identical to the amino acid sequence set forth in SEQ ID NO:25. In certain embodiments, the QRS polypeptide is truncated at its C-terminus. In certain embodiments, the QRS polypeptide comprises the amino acid sequence of SEQ ID NO:25, wherein at least about 1-50, 50-100, 50-100, 100-150, 150-200, 200-250, 250-300, 300-350, 350-400, 400-450, 450-500, or about 500-550 amino acid residues are truncated from its C-terminus. In certain embodiments, the QRS polypeptide comprises amino acid residues 1-183, 1-220, 1-249, or 1-200 of SEQ ID NO:25, or any one or more of SEQ ID NOS:36-108.

In certain embodiments, the aminoacyl-tRNA synthetase is a HisRS polypeptide. Certain embodiments comprise HisRS splice variant polypeptide. In certain embodiments, the HisRS polypeptide comprises at least the WHEP domain of HisRS. In certain embodiments, the HisRS polypeptide comprises at least the anticodon binding domain of HisRS. In certain embodiments, the HisRS polypeptide lacks a functional aminoacylation domain. In certain embodiments, the HisRS polypeptide comprises at least the WHEP domain of HisRS and the anticodon binding domain of HisRS but lacks a functional aminoacylation domain. In certain embodiments, the HisRS polypeptide comprises the sequence set forth in SEQ ID NO:28, 30, or 32. In certain embodiments, the HisRS polypeptide comprises an amino acid sequence at least 80%, 90%, 95%, 98%, or 100% identical to the amino acid sequence set forth in SEQ ID NO:28, 30, or 32. In certain embodiments, the HisRS polypeptide comprises at least 20 contiguous amino acid residues of the sequence set forth in SEQ ID NO:28, 30, or 32.

In certain embodiments, the aminoacyl-tRNA synthetase is a WRS polypeptide. In certain embodiments, the WRS polypeptide comprises an amino acid sequence at least 80%, 90%, 95%, 98%, or 100% identical to the amino acid sequence set forth in any one or more of SEQ ID NOS:33-35. In certain embodiments, the WRS polypeptide comprises a biologically active fragment of any one or more of SEQ ID NOS:33-35.

Certain embodiments include pharmaceutical compositions for modulating hematopoiesis in a subject, comprising an aminoacyl-tRNA synthetase (AARS) polypeptide as in any one of claims 1-51 and a pharmaceutically acceptable carrier.

Certain embodiments include methods of modulating hematopoiesis, comprising contacting a cell with an effective concentration of an aminoacyl-tRNA synthetase (AARS) polypeptide having a hematopoiesis-modulating activity, thereby modulating hematopoiesis. In certain embodiments, the cell is a stem cell or a progenitor cell. In certain embodiments, the progenitor cell is a megakaryocyte progenitor cell, an erythrocyte progenitor cell, a lymphocyte progenitor cell, a granulocyte progenitor cell, a monocyte, or an endothelial progenitor cell.

Certain embodiments comprise stimulating the production of at least one of megakaryocyte progenitor cells, erythrocyte progenitor cells, lymphocyte progenitor cells, granulocyte progenitor cells, or monocytes. Certain embodiments comprise stimulating the production of at least one of megakaryocytes, platelets, erythrocytes, lymphocytes, granulocytes, or macrophages. Certain embodiments comprise reducing the production of at least one of megakaryocyte progenitor cells, erythrocyte progenitor cells, lymphocyte progenitor cells, granulocyte progenitor cells, or monocytes. Certain embodiments comprise reducing the production of at least one of megakaryocytes, platelets, erythrocytes, lymphocytes, granulocytes, or macrophages.

Certain embodiments comprise contacting the cell in vitro or ex vivo. Certain embodiments comprise administering the contacted cells to a subject. Certain embodiments comprise contacting the cell in a subject by directly administering the AARS polypeptide to the subject.

In certain embodiments, the subject is about to undergo, is undergoing, or has undergone a transplant therapy. In certain embodiments, the transplant therapy is a bone marrow transplant, a cord blood transplant, a hematopoietic stem cell transplant, an autologous peripheral blood cell progenitor transplant, or a liver transplant.

In certain embodiments, the subject is about to undergo, is undergoing, or has undergone chemotherapy or radiotherapy. In certain embodiments, chemotherapy comprises treatment with an agent selected from chlorambucil, cyclophosphamide, lomustine (CCNU), melphalan, procarbazine, thiotepa, carmustine (BCNU), busulfan, daunorubicin, doxorubicin, idarubicin, epirubicin, mitoxantrone, bleomycin, cisplatin, carboplatin, oxaliplatin, camptothecins, irinotecan, topotecan, amsacrine, etoposide, etoposide phosphate, teniposide, vincristine, vinblastine, vinorelbine, vindesine, paclitaxel, mechlorethamine, ifosfamide, nitrosurea, dactinomycin, plicomycin, mitomycin, tamoxifen, raloxifene, estrogen receptor binding agents, gemcitabien, navelbine, farnesyl-protein transferase inhibitors, transplatinum, 5-fluorouracil, methotrexate, and temazolomide. In certain embodiments, chemotherapy comprises high-dose chemotherapy. In certain embodiments, high-dose chemotherapy further comprises a transplant therapy.

In certain embodiments, the transplant therapy is a bone marrow transplant, a cord blood transplant, a hematopoietic stem cell transplant, or an autologous peripheral blood cell progenitor transplant.

In certain embodiments, the subject has a condition that is associated with increased red blood cell count. In certain embodiments, the conditions is living at a high altitude, smoking, congenital heart disease, failure of the right side of the heart, pulmonary fibrosis, polycythemia vera, dehydration, kidney disease, kidney, cancer, exposure to carbon monoxide, anabolic steroid use, COPD, or erythropoietin (EPO) doping. In certain embodiments, the subject has a condition that is associated with decreased red blood count.

In certain embodiments, the subject has a condition that is associated with increased lymphocyte count. In certain embodiments, the subject has a condition that is associated with decreased lymphocyte count. In certain embodiments, the subject has a condition that is associated with increased granulocyte count. In certain embodiments, the subject has a condition that is associated with decreased granulocyte count. In certain embodiments, the subject has a condition that is associated with increased platelet count. In certain embodiments, the subject has a condition that is associated with decreased platelet count.

Certain embodiments include methods of modulating hematopoiesis in a subject, comprising administering to the subject an effective concentration of an aminoacyl-tRNA synthetase (AARS) polypeptide, thereby modulating hematopoiesis in the subject.

Certain embodiments comprise increasing the hematopoiesis-stimulatory activity of at least one of an osteoblast cell or a vascular cell. Certain embodiments comprise reducing the hematopoiesis-stimulatory activity of at least one of an osteoblast cell or a vascular cell. Certain embodiments comprise increasing the hematopoiesis-inhibitory activity of at least one of an osteoblast cell or a vascular cell. Certain embodiments comprise reducing the hematopoiesis-inhibitory activity of at least one of an osteoblast cell or a vascular cell.

Certain embodiments comprise increasing the mobilization of hematopoietic cells from the bone marrow to the periphery of the subject. In certain embodiments, the subject is a peripheral blood cell donor.

In certain embodiments, the subject is about to undergo, is undergoing, or has undergone a transplant therapy. In certain embodiments, the subject is about to undergo, is undergoing, or has undergone chemotherapy or radiotherapy. In certain embodiments, the subject has a condition that is associated with at least one of increased red blood cell, increased lymphocyte count, increased granulocyte count, or increased platelet count. In certain embodiments, the subject has a condition that is associated with at least one of decreased red blood cell count, decreased lymphocyte count, decreased granulocyte count, or decreased platelet count.

Also included are methods of co-administering to the subject one or more thrombopoiesis-stimulatory agents, such as thrombopoietin (TPO), a TPO agonist, a TPO mimetic, an mpl-signaling agonist, a cytokine, a chemokine, a chemokine receptor ligand, or an adhesion molecule. In some embodiments, the co-administration of the agent achieves a synergistic effect. In particular embodiments, the agent is TPO, a TPO agonist, a TPO mimetic, or an mpl-signaling agonist, and the synergistic effect comprises increased thrombopoiesis. In specific embodiments, the agent is eltrombopag or romiplostim. In certain embodiments, the AARS polypeptide is a YRS polyeptide comprising the sequence set forth in SEQ ID NO:2 (Y341A).

Also included are compositions, comprising an AARS polypeptide and one or more thrombopoiesis-stimulatory agents, such as thrombopoietin (TPO), a TPO agonist, a TPO mimetic, an mpl-signaling agonist, a cytokine, a chemokine, a chemokine receptor ligand, or an adhesion molecule. In some embodiments, the agent is TPO, a TPO agonist, a TPO mimetic, or an mpl-signaling agonist. In specific embodiments, the agent is eltrombopag or romiplostim. In certain embodiments, the AARS polypeptide is a YRS polyeptide comprising the sequence set forth in SEQ ID NO:2 (Y341A).

Certain embodiments include isolated polynucleotides encoding an AARS polypeptide provided herein. Certain embodiments include vectors comprising said polynucleotides. Certain embodiments include host cells comprising said vectors. Certain embodiments include fusion polypeptides comprising an AARS polypeptide provided herein and a heterologous fusion partner.

Also included are antibodies that exhibit binding specificity for an isolated YRS polypeptide of SEQ ID NO:2 or 3, a cellular binding partner of the YRS polypeptide, or both. In specific embodiments, affinity of the antibody for the YRS polypeptide of SEQ ID NO:2 or 3 is at least about 10× stronger than its affinity for the YRS polypeptide of SEQ ID NO:1.

Also included are binding agents that exhibit binding specificity for an isolated YRS polypeptide of SEQ ID NO:2 or 3, a cellular binding partner of the YRS polypeptide, or both. In specific embodiments, affinity of the binding agent for the YRS polypeptide of SEQ ID NO:2 or 3 is at least about 10× stronger than its affinity for the YRS polypeptide of SEQ ID NO:1. In certain embodiments, the binding agent is selected from a peptide, a soluble receptor, an adnectin, a small molecule, and an aptamer.

In some embodiments, the antibody or binding agent antagonizes a hematopoiesis-modulating activity of the YRS polypeptide of SEQ ID NO:2 or 3. In other embodiments, the antibody or binding agent agonizes a hematopoiesis-modulating activity of the YRS polypeptide of SEQ ID NO:2 or 3. In certain embodiments, the hematopoiesis-modulating is a thrombopoietic activity.

Also included are methods of reducing platelet count in a subject, comprising administering to the subject an antibody or binding agent that antagonizes the thrombopoietic activity of a YRS polypeptide, such as the YRS polypeptides of SEQ ID NOS:2 or 3, thereby reducing platelet count in the subject. In some embodiments, the subject has thrombocythemia or thrombocytosis. Specific embodiments include methods of reducing cell division of one or more platelets, comprising contacting the platelet(s) with an antibody or binding agent that antagonizes the thrombopoietic activity of a YRS polypeptide, such as the YRS polypeptides of SEQ ID NOS:2 or 3, thereby reducing cell division of the platelet(s).

As noted above, also included are methods of reducing platelet count in a subject, comprising administering to the subject an AARS polypeptide, wherein the AARS polypeptide reduces thrombopoiesis or megakaryopoiesis, thereby reducing platelet count in the subject. In some embodiments, the AARS polypeptide is a WRS polypeptide or a selected YRS polypeptide. In specific embodiments, the YRS polypeptide is a variant of SEQ ID NO:2 which reduces thrombopoiesis or megakaryopoiesis.

### Sequence Listing

SEQ ID NO:1 is the full-length amino acid sequence of human tyrosyl-tRNA synthetase (YRS).

SEQ ID NO:2 is the amino acid sequence of a Y341A variant of full-length human YRS.

SEQ ID NO:3 is the amino acid sequence of a C-terminally truncated (amino acids 1-364) human YRS.

SEQ ID NO:4 is a polynucleotide sequence that encodes the full-length amino acid sequence of human YRS (SEQ ID NO:1).

SEQ ID NO:5 shows the sequence of an eight amino acid tag.

SEQ ID NO:6 is the amino acid sequence of the SP1 human YRS splice variant.

SEQ ID NO:7 is the polynucleotide sequence that encodes the SP1 human YRS splice variant (SEQ ID NO:6).

SEQ ID NO:8 is the amino acid sequence of the SP2 human YRS splice variant.

SEQ ID NO:9 is the polynucleotide sequence that encodes the SP2 human YRS splice variant (SEQ ID NO:8)

SEQ ID NO:10 is the amino acid sequence of the SP3 human YRS splice variant.

SEQ ID NO:11 is the polynucleotide sequence that encodes the SP3 human YRS splice variant (SEQ ID NO:10).

SEQ ID NO:12 is the amino acid sequence of the SP4 human YRS splice variant.

SEQ ID NO:13 is the polynucleotide sequence that encodes the SP4 human YRS splice variant (SEQ ID NO:12).

SEQ ID NO:14 is the amino acid sequence of the SP5 human YRS splice variant.

SEQ ID NO:15 is the polynucleotide sequence that encodes the SP5 human YRS splice variant (SEQ ID NO:14).

SEQ ID NO:16 is the full length amino acid sequence of human cytoplasmic glycyl-tRNA synthetase (GlyRS).

SEQ ID NO:17 is a nucleic acid sequence encoding the GlyRS polypeptide of SEQ ID NO:16.

SEQ ID NOS:18-24 represent illustrative peptide sequences analyzed in determining GlyRS fragment boundaries (see Example 9 & Table 1).

SEQ ID NO:25 is the full-length amino acid sequence of human glutaminyl-tRNA synthetase (QRS).

SEQ ID NOS:26 and 27 represent illustrative peptide sequences analyzed in determining QRS fragment boundaries (see Example 10 & Table 2).

SEQ ID NO:28 is the full-length amino acid sequence of the histidyl-tRNA synthetase (HisRS) protein (NP_{—}002100.2).

SEQ ID NO:29 is a nucleic acid coding sequence of the HisRS-SV9 splice variant.

SEQ ID NO:30 is the amino acid sequence of the HisRS-SV9 splice variant polypeptide encoded by SEQ ID NO:29.

SEQ ID NO:31 is a nucleic acid coding sequence of the HisRS-SV11 splice variant.

SEQ ID NO:32 is the amino acid sequence of the HisRS-SV11 splice variant polypeptide encoded by SEQ ID NO:31.

SEQ ID NO:33 is the amino acid sequence of the main isoform of human tryptophanyl-tRNA synthetase (WRS).

SEQ ID NO:34 is the amino acid sequence of a fragment (T2) of human WRS.

SEQ ID NO:35 is the amino acid sequence of a fragment (Toltrup) of human WRS.

SEQ ID NOS:36-103 represent various endogenous peptide fragments of human QRS.

SEQ ID NO:104 is the amino acid sequence of a human phenylalanyl-tRNA synthetase (PheRS) splice variant (PheRS_SV1P).

SEQ ID NO:105 is the amino acid sequence of a full-length human aspartyl-tRNA synthetase (AspRS) polypeptide.

SEQ ID NO:106 is the amino acid sequence of an N-terminal fragment (F1; amino acids 1-471) of human WRS.

SEQ ID NO:107 is the amino acid sequence of a splice variant (mini-WRS; amino acids 48-471) of human WRS.

SEQ ID NO:108 is the amino acid sequence of a fragment (T1; amino acids 71-471) of human WRS).

### BRIEF DESCRIPTION OF THE DRAWINGS

FIG. 1 (A-B) shows the in vivo effects on platelet production following administration of a C-terminally truncated human-tyrosyl-tRNA synthetase polypeptide (SEQ ID NO:3) having an eight amino acid C-terminal tag, L-E-H-H-H-H-H-H (SEQ ID NO:5). FIG. 1(A) shows he platelet count for the experiment in which mice were injected with 1, 3, and 10 μg/kg of the truncated tyrosyl-tRNA synthetase polypeptide, as compared to a phosphate-buffer saline (PBS) control. FIG. 1(B) shows the platelet count for the experiment in which mice were injected with 3 μg/kg of the truncated tyrosyl-tRNA synthetase polypeptide, as compared to a PBS control. See Example 1.

FIG. 2 shows the in vivo effects on megakaryocyte number following administration of a C-terminally truncated human-tyrosyl-tRNA synthetase polypeptide (SEQ ID NO:3) having an eight amino acid C-terminal tag, L-E-H-H-H-H-H-H (SEQ ID NO:5). See Example 1.

FIG. 3 shows the in vivo effects on platelet production following administration of two YRS polypeptide variants, mini-YRS and Y341A.

FIG. 4 (A-D) shows the domain structure and amino acid sequence of GlyRS (SEQ ID NO:16), and illustrates the SDS-PAGE separation of fragments of GlyRS generated by controlled proteolysis of the full-length GlyRS protein with human neutrophil elastase. See Example 9.

FIG. 5 (A-C) shows the domain structure and amino acid sequence of QRS (SED ID NO:25), and illustrates the SDS-PAGE separation of fragments of QRS (SEQ ID Nos. 26 and 27) generated by endogenous or controlled proteolysis of the full-length QRS. See Example 10.

FIG. 6 shows the QRS fragments that were cloned into an E. coli protein expression vector for over-expression and purification. See Example 10.

FIG. 7 shows the impact of a variety of AARS polypeptide on the in vitro formation of the most primitive lineage-restricted (early) megakaryocyte progenitors (FIG. 7(A)), and the more mature (late) megakaryocyte progenitors (FIG. 7(B)). See Example 11.

FIG. 8 shows the impact of a variety of AARS polypeptides on the formation of erythroid progenitor cells (CFU-E). See Example 12.

FIG. 9 illustrates the alternate gene splicing of wild-type (WT) human tyrosyl-tRNA synthetase, as represented by the cDNA sequence of alternate splice variants SP1 to SP5.

FIG. 10 provides the NCBI annotation of the cDNA sequences for human tyrosyl-tRNA synthetase splice variants SP1 to SP5.

FIG. 11 depicts the protein sequence alignment of the predicted and reported open reading frames for the SP1 to SP5 YRS polypeptides as compared to the full-length human YRS polypeptide.

FIG. 12 illustrates mRNA transcripts of wild type, full length HisRS, HRS-SV9 and HRS-SV11. FIG. 12A shows an illustration of mRNA transcripts showing that HRS-SV9 has an insertion from Intron 2 and HRS-SV11 has a deletion from Exon 3 to Exon 10. FIG. 12B shows protein structural information encoded by the mRNA transcripts, showing that HRS-SV9 has only the first 60 amino acids of HisRS, including the intact WHEP domain, whereas HRS-SV11 has a deletion of the whole aminoacylation domain, leaving only the WHEP and anticodon domains.

### DETAILED DESCRIPTION

The present invention stems from the discovery that aminoacyl-tRNA synthetases (AARS) and certain polypeptides derived therefrom possess non-canonical biological activities of therapeutic relevance. Therefore, according to one aspect, the present invention provides isolated AARS polypeptides having at least one non-canonical biological activity, as well as active fragments and variants thereof which substantially retain said non-canonical activity.

“Non-canonical” activity,” as used herein, refers generally to an activity possessed by an AARS polypeptide of the invention that is other than the addition of an amino acid onto a tRNA molecule. As detailed herein, in certain embodiments, a non-canonical biological activity exhibited by an AARS polypeptide of the invention may include, but is not limited to, the modulation of hematopoiesis. Examples of hematopoietic-modulating activities include increasing or reducing any one or more of megakaryopoiesis, thrombopoiesis, erythropoiesis, granulopoiesis, or lymphopoiesis. Hence, embodiments of the present invention include aminoacyl-tRNA synthetase polypeptides, including truncations, splice variants, proteolytic fragments, and variants thereof, which regulate or modulate hematopoiesis, and thereby possess therapeutically beneficial activity.

Advantages of the use of AARS polypeptides over other treatments include, for example, a different mechanism of action than traditional treatments, synergism with hematopoietic-based signaling, higher potency, and the benefits associated with using a de-immunized molecule, which possibly possesses homeostasis functions. Other advantages will be apparent to a person skilled in the art.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); A Practical Guide to Molecular Cloning (B. Perbal, ed., 1984).

All publications, patents and patent applications cited herein are hereby incorporated by reference in their entirety.

### DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length.

An “agonist” refers to a molecule that intensifies or mimics a hematopoiesis-modulating activity of an AARS. Agonists may include antibodies and other binding agents such as proteins, nucleic acids, carbohydrates, small molecules, or any other compound or composition that modulates the activity of an AARS either by directly interacting with the AARS or its binding partner, or by acting on components of the biological pathway in which the AARS participates. Included are partial and full agonists.

The term “antagonist” refers to a molecule that reduces or attenuates a hematopoiesis-modulating activity of an AARS. Antagonists may include antibodies and other binding agents such as proteins, nucleic acids, carbohydrates, small molecules, or any other compound or composition that modulates the activity of an AARS or its binding partner, either by directly interacting with the AARS or its binding partner or by acting on components of the biological pathway in which the AARS participates. Included are partial and full antagonists.

The term “biologically active fragment”, as applied to fragments of a reference polynucleotide or polypeptide sequence, refers to a fragment that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity of a reference sequence. Included within the scope of the present invention are biologically active fragments of at least about 18, 19, 20, 21, 22, 23, 24, 25, 26, 27, 28, 29, 30, 40, 50, 60, 70, 80, 90, 100, 120, 140, 160, 180, 200, 220, 240, 260, 280, 300, 320, 340, 360, 380, 400, 420, 440, 460, 480, 500 or more contiguous nucleotides or amino acid residues in length, including all integers in between, which comprise or encode a hematopoietic-modulating activity of a reference amino-acyl tRNA transferase polynucleotide or polypeptide, such as the exemplary reference polypeptide sequences of SEQ ID NOS: 1, 2, 3, 6, 8, 10, 12, 14, 16, 25, 28, 30, and 32-108, or exemplary the reference nucleotide sequences of SEQ ID NOS: 4, 7, 9, 11, 13, 15, 17, 19, and 31.

Biologically active fragments also include naturally occurring splice variants of a reference AARS sequence, as well as proteolytic fragments of AARS polypeptides.

“Proteolytic fragments,” or the sequence of proteolytic fragments, can be identified or derived according to a variety of techniques. For instance, as exemplified herein, proteolytic fragments can be identified in vitro, such as by incubating QRS polypeptides with selected proteases, or they can be identified endogenously (i.e., in vivo). In certain embodiments, endogenous proteolytic fragments can be generated or identified, for instance, by recombinantly expressing QRS polypeptides in a selected microorganism or eukaryotic cell that has been either modified to contain one or more selected proteases, or that naturally contains one or more proteases that are capable of acting on a QRS polypeptide, and isolating and characterizing the endogenously produced proteolytic fragments therefrom. Examples of such proteolytic fragments include Q1-Q4, as described herein, as well as the proteolytic fragments illustrated in Tables C-G, including variants thereof.

In certain embodiments, naturally-occurring endogenous proteolytic fragments can be generated or identified, for instance, from various cellular fractions (e.g., cytosolic, membrane, nuclear) and/or growth medium of various cell-types, including, for example, macrophages such as RAW macrophages (e.g., RAW 264.7 macrophages; see Example 5), T-cells, including primary T-cells and T-cell lines such as Jurkats, and natural killer (NK) cells, among others. In certain embodiments, endogenous proteolytic fragments, however generated, can be identified by techniques such as mass-spectrometry, or equivalent techniques. Once an in vitro or endogenously identified proteolytic fragment has been generated or identified, then it can be sequenced and cloned into an expression vector for recombinant production, or produced synthetically.

Representative biologically active fragments generally participate in an interaction, e.g., an intramolecular or an inter-molecular interaction. An inter-molecular interaction can be a specific binding interaction or an enzymatic interaction. An inter-molecular interaction can be between an AARS polypeptide and target molecule, such as another AARS polypeptide or a target molecule involved in modulating the process of hematopoiesis (e.g., megakaryopoiesis, erythropoiesis). Biologically active fragments of an AARS polypeptide include polypeptide fragments comprising amino acid sequences with sufficient similarity or identity to, or which are derived from, the amino acid sequences of any of SEQ ID NOS: 1, 2, 3, 6, 8, 10, 12, 14, 16, 25, 28, 30, or 32-108, including biologically active portions thereof, or are encoded by a nucleotide sequences of SEQ ID NOS: 4, 7, 9, 11, 13, 15, 17, 19, or 31.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties (e.g., pegylation) or by post-translational modification techniques as would be understood in the art. The term “derivative” also includes within its scope alterations that have been made to a parent sequence including additions or deletions that provide for functionally equivalent molecules.

As used herein, the terms “function” and “functional” and the like refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (Deveraux et al., 1984, Nucleic Acids Research 12, 387-395), which is incorporated herein by reference. In this way sequences of a similar or substantially different length to those cited herein could be compared by insertion of gaps into the alignment, such gaps being determined, for example, by the comparison algorithm used by GAP.

The term “host cell” includes an individual cell or cell culture that can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a recombinant host cell.

By “isolated” is meant material that is substantially or essentially free from components that normally accompany it in its native state. For example, an “isolated polynucleotide,” as used herein, includes a polynucleotide that has been purified from the sequences that flank it in its naturally-occurring state, e.g., a DNA fragment which has been removed from the sequences that are normally adjacent to the fragment. Alternatively, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances.

By “obtained from” is meant that a sample such as, for example, a polynucleotide extract or polypeptide extract is isolated from, or derived from, a particular source of the subject. For example, the extract can be obtained from a tissue or a biological fluid isolated directly from the subject.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

The term “operably linked” as used herein means placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e., the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived.

The recitation “polynucleotide” or “nucleic acid” as used herein designates mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

The terms “polynucleotide variant” and “variant” and the like refer to polynucleotides displaying substantial sequence identity with a reference AARS polynucleotide sequence or polynucleotides that hybridize to an AARS reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. Polynucleotide variants include, for example, polynucleotides having at least 50% (and at least 51% to at least 99% and all integer percentages in between) sequence identity with the sequence set forth in SEQ ID NO:4, 7, 9, 11, 13, 15, 17, 19, or 31, or portions thereof that encode a biologically active fragment of an AARS polypeptide. The terms “polynucleotide variant” and “variant” also include naturally occurring allelic variants.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The term “aminoacyl-tRNA synthetase” (AARS) refers generally to enzymes that in their natural or wild-type form are capable of catalyzing the esterification of a specific amino acid or its precursor to one of all its compatible cognate tRNAs to form an aminoacyl-tRNA. In this “canonical” activity, aminoacyl-tRNA synthetases catalyse a two-step reaction: first, they activate their respective amino acid by forming an aminoacyl-adenylate, in which the carboxyl of the amino acid is linked in to the alpha-phosphate of ATP by displacing pyrophosphate, and then, when the correct tRNA is bound, the aminoacyl group of the aminoacyl-adenylate is transferred to the 2′ or 3′ terminal OH of the tRNA.

Class I aminoacyl-tRNA synthetases typically have two highly conserved sequence motifs. These enzymes aminoacylate at the 2′-OH of an adenosine nucleotide, and are usually monomeric or dimeric. Class II aminoacyl-tRNA synthetases typically have three highly conserved sequence motifs. These enzymes aminoacylate at the 3′-OH of the same adenosine, and are usually dimeric or tetrameric. The active sites of class II enzymes are mainly made up of a seven-stranded anti-parallel β-sheet flanked by α-helices. Although phenylalanine-tRNA synthetase is class II, it aminoacylates at the 2′-OH.

AARS polypeptides include tyrosyl-tRNA synthetases (YRS), tryptophanyl-tRNA synthetases (WRS), glutaminyl-tRNA synthetases (QRS), glycyl-tRNA synthetases (GlyRS), histidyl-tRNA synthetases, seryl-tRNA synthetases, phenylalanyl-tRNA synthetases, alanyl-tRNA synthetases, asparaginyl-tRNA synthetases (AsnRS), aspartyl-tRNA synthetases (AspRS), cysteinyl-tRNA synthetases (CysRS), glutamyl-tRNA synthetases, prolyl-tRNA synthetases (ProRS), arginyl-tRNA synthetases, isoleucyl-tRNA synthetases, leucyl-tRNA synthetases, lysyl-tRNA synthetases, threonyl-tRNA synthetases, methionyl-tRNA synthetases, and valyl-tRNA synthetases. The full-length wild-type sequences of these AARS polypeptides are known in the art. Also included within the meaning of AARS polypeptides are aminoacyl tRNA synthetase-interacting multifunctional proteins (AIMPs), including AIMP-1 (or p43), AIMP-2 (or p38), and AIMP-3 (or p18).

The recitations “polypeptides” “polypeptide fragments,” “truncated polypeptides” or “variants thereof” encompass, without limitation, polypeptides having the amino acid sequence that shares at least 50% (and at least 51% to at least 99% and all integer percentages in between) sequence identity with a reference AARS sequence, such as the amino acid sequence of a human AARS polypeptide, including biologically active fragments thereof, such as fragments having at least about 10, 20, 30, 40, 50, 60, 70, 80, 90, 100, 120, 140, 160, 180, 200, 220, 240, 260, 280, 300, 350, 400, 450, 500, 550, 600, 650, 700 or more contiguous amino acids of the reference sequences, including all integers in between. These recitations further encompass natural allelic variation of AARS polypeptides that may exist and occur from one genus or species to another. Illustrative reference sequences include those set forth in any one of SEQ ID NOS: 1, 2, 3, 6, 8, 10, 12, 14, 16, 25, 28, 30, and 32-108.

AARS polypeptides, including truncations, fragments, and/or variants thereof, encompass polypeptides that exhibit at least about 10%, 20%, 30%, 40%, 50%, 60%, 70%, 80%, 90%, 100%, 110%, 120%, 130%, 140%, 150%, 200%, 300%, 400%, 500%, 600%, 700%, 800%, 900%, 1000% or more of the specific biological activity of a reference AARS polypeptide (e.g., a hematopoietic-modulating activity in a subject or in vitro). Merely by way of illustration, AARS-related biological activity may be quantified, for example, by measuring the ability of an AARS polypeptide to increase the megakaryocyte number in a subject (see, e.g., Example 1), or to impact the formation of megakaryocyte progenitor cells (see, e.g., Example 11). Suitable in vitro models for assaying megakaryocyte colony formation are described herein, and exemplified in Dessypris et al., Exp Hematol. 18:754-7, 1990. AARS polypeptides, including truncations and/or variants thereof, having substantially reduced biological activity relative to a reference AARS polypeptide are those that exhibit less than about 25%, 10%, 5% or 1% of the specific activity of a reference AARS polypeptide (i.e., having a non-canonical biological activity).

The recitation polypeptide “variant” refers to polypeptides that are distinguished from a reference polypeptide by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The present invention contemplates the use in the methods described herein of variants of full-length AARS polypeptides (e.g., a full-length YRS polypeptide having a Y341A substitution), truncated fragments of full-length AARS polypeptides, splice variants, proteolytic fragments, including endogenous proteolytic fragments, and variants of such fragments, as well as their related biologically active fragments. Biologically active fragments of an AARS polypeptide include peptides comprising amino acid sequences sufficiently similar to, or derived from, the amino acid sequences of a (putative) full-length AARS polypeptide sequence, such as SEQ ID NO:1, or portions thereof, such as the polypeptides of SEQ ID NOS: 3, 6, 8, 10, 12, and 14.

Typically, biologically active fragments comprise a domain or motif with at least one activity of an AARS polypeptide and may include one or more (and in some cases all) of the various active domains, and include fragments having a hematopoietic-modulating activity. In some cases, biologically active fragments of an AARS polypeptide have a biological activity (e.g., megakaryopoiesis-modulating activity, erythropoiesis-modulating activity) that is unique to the particular, truncated fragment, such that the full-length AARS polypeptide may not have that activity. In certain cases, the biological activity may be revealed by separating the biologically active AARS polypeptide fragment from the other full-length AARS polypeptide sequences, or by altering certain residues (e.g., Y341A of the YRS polypeptide) of the full-length AARS wild-type polypeptide sequence to unmask the biologically active domains. A biologically active fragment of a truncated AARS polypeptide can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 550, 600, 650, 700, 750 or more contiguous or non-contiguous (e.g., splice variants may not be contiguous) amino acids, including all integers in between, of the amino acid sequences set forth in any one of SEQ ID NOS: 1, 2, 3, 6, 8, 10, 12, 14, 16, 25, 28, 30, or 32-108, or the known amino acid sequences of the various human AARS polypeptides. In certain embodiments, a biologically active fragment comprises a hematopoiesis modulating sequence, domain, or motif. Suitably, the biologically-active fragment has no less than about 1%, 10%, 25%, or 50% of an activity of the biologically-active (i.e., non-canonical activity) polypeptide from which it is derived.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology,” John Wiley & Sons Inc, 1994-1998, Chapter 15.

A “subject,” as used herein, includes any animal that exhibits a symptom, or is at risk for exhibiting a symptom, which can be treated with either an AARS polypeptide of the invention, cells (e.g., bone marrow stem cells) that have been treated ex vivo or in vitro with an AARS polypeptide, such as to increase the proportion or number of megakaryocyte progenitors, or both. Suitable subjects (patients) include laboratory animals (such as mouse, rat, rabbit, or guinea pig), farm animals, and domestic animals or pets (such as a cat or dog). Non-human primates and, preferably, human patients, are included. Typical subjects include animals that exhibit, or are at risk for exhibiting increased or reduced platelet count, increased or reduced red blood cell count, increased or reduced lymphocyte or granulocyte count, or other hematopoietic-related symptom. As one example, a “subject” may also be about to undergo, is undergoing, or has undergone, a transplant procedure, such as a stem cell or bone marrow transplant.

An “effective concentration” of an aminoacyl-tRNA synthetase polypeptide refers to an amount that is capable of modulating or regulating hematopoiesis in any desired way, as compared to a control polypeptide or no polypeptide, whether in one or more cells in vitro or ex vivo, or in subject. One example of a hematopoiesis-modulating activity includes increasing megakaryopoiesis, as typically measured by increased platelet levels, maintained platelet levels, increased megakaryocyte numbers, and/or increased neutrophil production. Another example of a hematopoiesis-modulating activity includes reducing or increasing erythropoiesis, as typically measured by the formation of erythroid progenitor cells or erythrocytes (red blood cells). Other examples will be apparent from the description provided herein and the understanding in the art.

A “megakaryocyte” refers generally to a bone marrow cell that is responsible for the production of blood thrombocytes (i.e., platelets), which are necessary for normal blood clotting. Megakaryocytes typically account for 1 out of 10,000 bone marrow cells. Megakaryocytes are derived from pluripotent hematopoietic stem cell precursor cells in the bone marrow. Thrombopoietin (TPO) is the primary regulator of megakaryocyte production, i.e., TPO is sufficient but not absolutely necessary for inducing differentiation of progenitor cells in the bone marrow towards a final megakaryocyte phenotype. Other factors regulating megakaryocyte differentiation include GM-CSF, IL-3, IL-6, IL-11, chemokines (SDF-1; FGF-4), and erythropoietin.

Megakaryocytes are believed to develop through the following lineage: CFU-Me (pluripotential hematopoietic stem cell or hemocytoblast)-> megakaryoblast-> promegakaryocyte-> megakaryocyte. At the megakaryoblast stage, the cell loses its ability to divide, but is still able to replicate its DNA and continue development, becoming polyploid. Upon maturation, megakaryocytes begin the process of producing platelets, or thrombocytes. Thrombopoietin plays a role in inducing the megakaryocyte to form small proto-platelet processes, or cytoplasmic internal membranes for storing platelets prior to release. Upon release, each of these proto-platelet processes can give rise to 2000-5000 new platelets. Overall, about ⅔ of the newly-released platelets will remain in circulation and about ⅓ will be sequestered by the spleen. After releasing the platelets, the remaining cell nucleus typically crosses the bone marrow barrier to the blood and is consumed in the lung by alveolar macrophages. Megakaryocytopenia, also referred to as megakaryophthisis, is a scarcity of megakaryocytes in the bone marrow.

An “erythrocyte” refers to a red blood cell that consists mainly of hemoglobin, a complex metalloprotein containing heme groups whose iron atoms temporarily link to oxygen molecules (O₂) in the lungs. Erythrocytes are produced by a process called erythropoiesis, in which they develop from committed stem cells through reticulocytes to mature erythrocytes in about 7 days and live a total of about 100-120 days. “Polycythemias” (or erythrocytoses) are diseases characterized by a surplus of erythrocytes, in which the increased viscosity of the blood can cause a number of symptoms. “Anemias” are diseases characterized by low oxygen transport capacity of the blood, because of low red cell count or some abnormality of the red blood cells or the hemoglobin.

A “granulocyte” refers to a white blood cell that is characterized by the presence of granules in its cytoplasm. Granulocytes are also referred to as polymorphonuclear leukocytes (PMN or PML), because of the varying shapes of the nuclei. Examples of granulocytes include neutrophils, eosinophils, and basophils.

A “neutrophil,” or neutrophil granulocyte, refers generally to an abundant type of white blood cells in humans, which, together with basophils and eosinophils, form part of the polymorphonuclear cell family (PMNs). Neutrophils can be readily identified according to their unique staining characteristics on hematoxylin and eosin (H&E) histological or cytological preparations. Neutrophils are normally found in the blood stream, but are one of the first group of inflammatory cells to migrate toward inflammation sites during the beginning (i.e., acute) phase of inflammation, mainly as a result of infection or cancer. Typically, neutrophils first migrate through the blood vessels, and then through interstitial tissues, following chemical signals (e.g., interleukin-8 (IL-8), interferon-gamma (IFN-gamma), and C5a) that originate at the site of inflammation. “Neutropenia” refers to the presence of low neutrophil counts, which may result from a congenital (genetic) disorder, or may develop due to other conditions, as in the case of aplastic anemia or some kinds of leukemia. “Neutrophilia” refers to an abnormally high neutrophil count.

“Eosinophils,” also called eosinophilic leukocytes, refer to leukocytes that have coarse round granules of uniform size within their cytoplasm, and which typically have a bilobate (two-lobed) nucleus. The cytoplasmic granules of eosinophils stain red with the dye eosin. Eosinophils normally constitute about 1% to about 3% of the peripheral blood leukocytes, at a count of about 350 to 650 per cubic millimeter. Eosinophil counts in blood often rise above the normal range during allergic reactions and parasitic infections, such as worms. “Eosinopenia” refers to a form of agranulocytosis in which the number of eosinophil granulocyte is lower than expected. “Eosinophilia” refers to an abnormally high number of eosinophils in the blood. For example, eosinophilia can be categorized as mild (less than about 1500 eosinophils per cubic millimeter), moderate (about 1500 to about 5000 per cubic millimeter), or severe (more than about 5000 per cubic millimeter). In primary eosinophilia, the increased production of eosinophils is typically due to an abnormality in hematopoietic stem cells, such as in eosinophilic leukemia. In secondary eosinophilia, the increased production of eosinophils is typically due to a reactive process driven by cytokines.

Basophils, also called basophilic leukocytes, refer to leukocytes that have coarse bluish-black granules of uniform size within the cytoplasm, and which typically have a bilobate (two-lobed) nucleus. The cytoplasmic granules of basophils stain with basic dyes. Basophils normally constitute about 0.5% to 3% of the peripheral blood leukocytes. Basophils store and release histamine and serotonin, among other chemicals. Basophils are capable of ingesting foreign particles, and also produce, store and release heparin, serotonin, and histamine. The release of inflammatory chemicals such as heparin and histamine is often associated with asthma and allergies. Basophils are produced continually by stem cells in the bone marrow. “Basopenia” refers to a low basophil count (e.g., less than about 0.01×10⁹ per liter of blood), and “basophilia” refers to a high basophil count (e.g., more than about 10¹⁰ per liter of blood).

“Lymphocytes” refer generally to white blood cells of the vertebrate immune system, and include B-cells, T-cells (e.g., helper T-cells, cytotoxic T-cells, γδT-cells), and natural killer (NK) cells. Generally, and merely for illustrative purposes, B-cells produce and secrete antibodies, T-helper cells release cytokines and growth factors that regulate other immune cells, cytotoxic T-cells (CTLs) lyse virally infected cells, tumour cells and allografts, and NK cells lyse virally infected cells and tumour cells. “Lymphocytopenia” is characterized by abnormally low level of lymphocytes in the blood. The normal total lymphocyte count is typically about 1000 to 4800/μL in adults, and about 3000 to 9500/μL in children younger than 2 years. At age 6, the lower limit of normal total lymphocyte count is about 1500/μL. Lymphocytopenia is often characterized by a total lymphocyte count of <1000/μL in adults or <3000/μL in children younger than 2 years. Specific examples of lymphocytopenia include T-lymphocytopenia, in which there are too few T-cells (e.g., CD4+ T-cell counts below about 300 cells/μL) but often normal numbers of other lymphocytes, B lymphocytopenia, in which there are too few B lymphocytes but often normal numbers of other lymphocytes, and NK lymphocytopenia, in which there are there are too few natural killer cells but often normal numbers of other lymphocytes.

“Lymphocytosis” refers to an abnormally high lymphocyte count, often characterized by a total lymphocyte count that is more than 40% above normal. In adults, absolute lymphocytosis is typically present when the absolute lymphocyte count is greater than 4000 per microliter, in older children greater than 7000 per microliter, and in infants greater than 9000 per microliter. Relative lymphocytosis may occur when there is a higher proportion (greater than 40%) of lymphocytes among the white blood cells, and when lymphocyte count (ALC) is normal (less than about 4000 per microliter).

The term “modulating” includes “increasing” or “stimulating,” as well as “decreasing” or “reducing,” typically in a statistically significant or a physiologically significant amount.

The terms “enhance” or “enhancing,” or “increase” or “increasing,” or “stimulate” or “stimulating,” refer generally to the ability of one or agents or compositions to produce or cause a greater physiological response (i.e., downstream effects) in a cell, as compared to the response caused by either no AARS polypeptide or a control molecule/composition. A measurable physiological response may include greater cell growth, expansion, migration, or faster differentiation, among others apparent from the understanding in the art and the description herein. Among other methods known in the art, in vitro colony formation assays represent one way to measure cellular responses to agents provided herein. An “increased” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1), e.g., 1.5, 1.6, 1.7. 1.8, etc.) the amount produced by no AARS polypeptide (the absence of an agent) or a control composition.

The term “reduce” may relate generally to the ability of one or more AARS polypeptides of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art. A “decrease” in a response may be statistically significant as compared to the response produced by no AARS polypeptide or a control composition, and may include, for example, a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

“Migration” refers to cellular migration, a process that can be measured according to routine in vitro assays, as described herein and known in the art (see, e.g., Example 8). Migration also refers to in vivo migration, such as the migration of cells from one tissue to another tissue (e.g., from bone marrow to peripheral blood, or from peripheral blood to lung tissue), or from a site within one tissue to another site within the same tissue. Migration in vivo (e.g., chemotaxis) often occurs in a response to infection or damaged/irritated tissue.

“Differentiation” refers to the process by which a less specialized (e.g., pluripotent, totipotent, multipotent, etc.) cell becomes a more specialized cell type.

“Treatment” or “treating,” as used herein, includes any desirable effect on the symptoms or pathology of a disease or condition associated with the modulation of hematopoiesis (e.g., reduced platelet levels), or on the outcome of other primary treatments (e.g., transplants) that may benefit from the modulation of hematopoiesis, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any animal in need, including primates, in particular humans, and other mammals such as equines, cattle, swine and sheep; and poultry and pets in general. Also included are “prophylactic” treatments, which reduce the risk of developing a relevant disease or condition, or of developing symptoms associated with the disease or condition. Exemplary markers of clinical improvement include either increased platelet counts, maintenance of normal platelet counts, and/or increased megakaryocyte numbers, whether following administration of an AARS polypeptide, following administration of cells that have been treated ex vivo or in vitro with an AARS polypeptide, or both. Other exemplary clinical markers will be apparent to persons skilled in the art.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably one which is operably functional in a bacterial cell. The vector can also include a selection marker such as an antibiotic resistance gene that can be used for selection of suitable transformants.

The terms “wild-type” and “naturally occurring” are used interchangeably to refer to a gene or gene product that has the characteristics of that gene or gene product when isolated from a naturally occurring source. A wild-type gene or gene product (e.g., a polypeptide) is that which is most frequently observed in a population and is thus arbitrarily designed the “normal” or “wild-type” form of the gene.

### Aminoacyl-tRNA Polypeptides and Variants Thereof

The present invention relates in part to the unexpected observation that aminoacyl-tRNA synthetase (AARS) polypeptides, including truncations and variants thereof, modulate the hematopoietic process both in vivo and ex vivo (or in vitro). Accordingly, polypeptides of the present invention include full-length aminoacyl-tRNA synthetases, including any biologically active fragments, variants, or modifications thereof, wherein the polypeptides are capable of modulating hematopoiesis, such as by modulating the differentiation or growth of cells from the myeloid, megakaryocyte, erythrocyte, lymphoid and/or endothelial progenitor (EPC) lineages.

Aminoacyl-tRNA synthetases typically catalyze the aminoacylation of tRNA with their cognate amino acid. Because of their central role in linking amino acids with nucleotide triplets contained in tRNAs, aminoacyl-tRNA synthetases are thought to be among the first proteins that appeared in evolution.

As noted above, examples of aminoacyl-tRNA synthetases include tyrosyl-tRNA synthetases (YRS), tryptophanyl-tRNA synthetases (WRS), glutaminyl-tRNA synthetases (QRS), glycyl-tRNA synthetases (GlyRS), histidyl-tRNA synthetases (HisRS), seryl-tRNA synthetases (SRS), phenylalanyl-tRNA synthetases (PheRS), alanyl-tRNA synthetases (AlaRS), asparaginyl-tRNA synthetases (AsnRS), aspartyl-tRNA synthetases (AspRS), cysteinyl-tRNA synthetases (CysRS), glutamyl-tRNA synthetases (ERS), prolyl-tRNA synthetases (ProRS), arginyl-tRNA synthetases (RRS), isoleucyl-tRNA synthetases (IRS), leucyl-tRNA synthetases (LRS), lysyl-tRNA synthetases (KRS), threonyl-tRNA synthetases (TRS), methionyl-tRNA synthetases (MRS), and valyl-tRNA synthetases (VRS).

Tyrosyl-tRNA synthetases belong to the class I tRNA synthetase family, which has two highly conserved sequence motifs at the active site, HIGH and KMSKS. Class I tRNA synthetases aminoacylate at the 2′-OH of an adenosine nucleotide, and are usually monomeric or dimeric (one or two subunits, respectively).

The human tyrosyl-tRNA synthetase is composed of three domains: 1) an amino-terminal Rossmann fold domain that is responsible for formation of the activated E·Tyr-AMP intermediate and is conserved among bacteria, archeae, and eukaryotes; 2) a tRNA anticodon recognition domain that has not been conserved between bacteria and eukaryotes; and 3) a carboxyl-terminal domain that is unique to the human tyrosyl-tRNA synthetase, and whose primary structure is 49% identical to the putative human cytokine endothelial monocyte-activating protein II, 50% identical to the carboxyl-terminal domain of methionyl-tRNA synthetase from Caenorhabditis elegans, and 43% identical to the carboxyl-terminal domain of Arc1p from Saccharomyces cerevisiae.

The first two domains of the human tyrosyl-tRNA synthetase are 52, 36, and 16% identical to tyrosyl-tRNA synthetases from S. cerevisiae, Methanococcus jannaschii, and Bacillus stearothermophilus, respectively. Nine of fifteen amino acids known to be involved in the formation of the tyrosyl-adenylate complex in B. stearothermophilus are conserved across all of the organisms, whereas amino acids involved in the recognition of tRNA^{Tyr} are not conserved. Kinetic analyses of recombinant human and B. stearothermophilus tyrosyl-tRNA synthetases expressed in Escherichia coli indicate that human tyrosyl-tRNA synthetase aminoacylates human but not B. stearothermophilus tRNA^{Tyr}, and vice versa. It is believed that the carboxyl-terminal domain of human tyrosyl-tRNA synthetase evolved from gene duplication of the carboxyl-terminal domain of methionyl-tRNA synthetase and may direct tRNA to the active site of the enzyme.

Biological fragments of eukaryotic tyrosyl-tRNA synthetases connect protein synthesis to cell-signaling pathways, such as megakaryopoiesis. These fragments may be produced naturally by either alternative splicing or proteolysis, or by artificial proteolytic treatment. For example, as provided in the present invention, the N-terminal fragment mini-YRS is capable of modulating hematopoiesis in vivo. In addition, certain mutations in the full-length YRS polypeptide sequence confer increased hematopoietic-modulating activity on the wild-type reference sequence (e.g., Y341A). Examples of truncated splice variants of the full-length YRS polypeptide sequence include the SP1-SP5 polypeptides.

The full-length amino acid sequence of human tyrosyl-tRNA synthetase is set forth in SEQ ID NO:1. The structure of human mini-YRS (i.e., SEQ ID NO:3; or mini-Tyr), which contains both the catalytic and the anticodon recognition domain, has been reported to a resolution of 1.18 Å. Whereas the catalytic domains of the human and bacterial enzymes superimpose, the spatial disposition of the anticodon recognition domain relative to the catalytic domain is unique in mini-YRS relative to the bacterial orthologs. Without wishing to be bound by any one theory, the unique orientation of the anticodon-recognition domain may explain why the fragment mini-YRS is more active in various cell-signaling pathways.

Specific examples of YRS polypeptide variants include full-length YRS polypeptides, or truncations or splice variants thereof, having one or more amino acid substitutions selected from an R93Q substitution, an I14L substitution, an N17G substitution, an L271 substitution, an A85S substitution, and a V156L substitution, in addition to combinations thereof. Particular examples of YRS polypeptide variants include, but are not limited to, a YRS polypeptide having amino acids 1-364 of SEQ ID NO:1 with an R93Q substitution, a YRS polypeptide having amino acids 1-353 of SEQ ID NO:1 with an I14L substitution, a YRS polypeptide having aminoacids 1-353 of SEQ ID NO:1 with an N17G substitution, a YRS polypeptide having amino acids 1-353 of SEQ ID NO:1 with an L27I substitution, a YRS polypeptide having amino acids 1-353 of SEQ ID NO:1 with an A85S substitution, and a YRS polypeptide having amino acids 1-353 of SEQ ID NO:1 with a V156L substitution.

Particular examples of biologically active YRS fragments include, but are not limited to, C-terminally truncated tyrosyl-tRNA synthetase polypeptides comprising or consisting of amino acids 1-343, amino acids 1-344, amino acids 1-350, amino acids 1-353, or amino acids 1-364 of the amino acid sequence set forth in SEQ ID NO:1, in addition to the polypeptides of SEQ ID NOS:3 and 6. Additional examples of biologically active fragments include, but are not limited to, N-terminally truncated tyrosyl-tRNA synthetase polypeptides comprising or consisting of the amino acid sequences set forth in SEQ ID NOS: 6, 10, 12, and 14. These and other YRS polypeptides are included within the AARS polypeptides of the present invention.

Histidyl-tRNA synthetases (HisRS) are α2 dimers that belong to the class IIa tRNA synthetase family. A compilation of primary structures of HisRSs shows that the subunits of these homo-dimeric enzymes consist of 420-550 amino acid residues. This represents a relatively short chain length among AARSs, whose peptide chain sizes range from about 300 to 1100 amino acid residues. SEQ ID NO:28 is the amino acid sequence of the full length HisRS protein (NP_{—}002100.2). SEQ ID NO:30 is the amino acid sequence of the HRS-SV9 splice variant, and SEQ ID NO:32 is the amino acid sequence of the HRS-SV11 splice variant.

Examples of histidyl-tRNA synthetase polypeptides, and variants or truncations thereof, include HisRS fragments comprising at least the WHEP domain of HisRS, e.g., amino acid residues 3-43 of the human full length HisRS protein and HisRS fragments comprising at least the anticodon binding domain of HisRS, e.g., amino acid residues 406-501 of the full length human HisRS protein. Further examples include HisRS fragments that lack a functional aminoacylation domain, e.g., amino acid residues 54-398 of the human full length HisRS protein or HisRS splice variant polypeptides that comprise at least the WHEP domain and the anticodon binding domain but lack a functional aminoacylation domain.

In certain embodiments, the HisRS polypeptide of the invention comprises a sequence set forth in SEQ ID NOS:28, 30, or 32, or is a contiguous or non-contiguous (e.g., splice variants are sometimes non-contiguous) fragment of a polypeptide set forth in SEQ ID NOS:28, 30, or 32. Illustratively, the fragments may be of essentially any length, provided they retain at least one non-canonical biological activity of interest. For example, as further described herein, such a fragment may comprise at least about 5, 10, 15, 20, 25, 50, 75 or 80, or more, contiguous amino acid residues of SEQ ID NOS:28, 30, or 32.

In further embodiments of the invention, an HisRS polypeptide comprises an active variant (i.e., retains at least one non-canonical biological activity of interest) of a sequence set forth in SEQ ID NOS:28, 30, or 32. In certain embodiments, the active variant is a polypeptide having at least 70%, 80%, 90%, 95% or 99% identity along its length to a sequence set forth in SEQ ID NOS:28, 30, or 32. In certain embodiment, the HisRS polypeptide of the invention is not a polypeptide consisting of residues 1-48 of the full length human HisRS protein. These and other HisRS polypeptides are included within the AARS polypeptides of the present invention.

Tryptophanyl-tRNA synthetases (WRS), also referred to as tryptophan-tRNA ligases, belong to the class I tRNA synthetase family. Tryptophanyl-tRNA synthetase catalyzes the aminoacylation of tRNA^{trp} with tryptophan, an essential function in protein synthesis. Human WRS has a kinase domain in the N-terminal region and a serine phosphorylation site near the C-terminus.

Two main forms of human tryptophanyl-tRNA synthetase are produced in vivo through alternative mRNA splicing, to yield the full-length protein (SEQ ID NO: 33), and a fragment thereof, often designated mini-WRS (SEQ ID NO:107). Also included are human T1-WRS (SEQ ID NO:108) and T2-WRS (SEQ ID NO:34), alternate splice variants that are produced from an IFN-gamma-sensitive promoter, the latter being an N-terminally truncated fragment of WRS, as well as an N-terminal fragment (F1; SEQ ID NO:106) and fragment of WRS referred to as “Tolstrup” (SEQ ID NO:35). Other splice variants of human WRS are known in the art (see, e.g., Liu et al., Nucleic Acids Research, 32(2):719-27, 2004, herein incorporated by reference).

Structurally, full-length WRS contains three parts, a canonical dinucleotide-binding fold, a dimer interface, and a helical domain. This enzyme has enough structural homology to tyrosyl-tRNA synthetase (YRS) that the two enzymes can be described as conformational isomers. Structural elements interacting with the activated amino acid, tryptophanyl-5′ AMP, are almost exactly as seen in the tyrosyl-5′ AMP complex. Also, side chains that recognize indole are also highly conserved, and require reorientation of a “specificity-determining” helix containing a conserved aspartate to assure selection of tryptophan versus tyrosine. The carboxy terminus, which is disordered and therefore not seen in YRS, forms part of the dimer interface in WRS (see Doublie et al., Structure. 3:17-31, 1995).

The crystal structure of human T2-WRS has been reported at 2.5 Å resolution. This variant shares a very low sequence homology of 22% with Bacillus stearothermophilus WRS (bWRS), however their overall structures are strikingly similar. Structural comparison of T2-WRS with bWRS reveals substantial structural differences in the substrate-binding pocket and at the entrance to the pocket that play important roles in substrate binding and tRNA binding. T2-WRS has a wide opening to the active site and adopts a compact conformation similar to the closed conformation of bWRS. Modeling studies indicate that tRNA binds with the dimeric enzyme and interacts primarily with the connective polypeptide 1 of human WRS via its acceptor arm and the α-helical domain of WRS via its anticodon loop.

The amino acid sequence of the full-length WRS polypeptide (or the main splice variant) is shown in SEQ ID NO:33. The amino acid sequence of various splice variants or fragments are shown in SEQ ID NOS:34-35 and 106-108 Accordingly, these and other variants or fragments of WRS polypeptides are included within the AARS polypeptides of the present invention.

Glutaminyl-tRNA synthetases (QRS) belong to the class I tRNA synthetase family, and the human protein is one of several mammalian aminoacyl-tRNA synthetases that form a macromolecular protein complex. The eukaryote-specific N-terminal appendix of QRS appears to stabilize the association of other components in the multi-ARS complex, whereas the C-terminal catalytic domain is necessary for QRS association with the multi-AARS complex.

The human QRS enzyme differs from both the bacterial and yeast enzymes, suggesting that a considerable part of human QRS has evolved to perform functions other than the charging of tRNA. For instance, at least two distinct regions (part I and part II) within the eukaryotic QRS (EC 6.1.1.18) N-terminal region have no counterpart in Escherichia coli. Even though these regions are thought to bind RNA in a non-specific manner, enhancing interactions between the tRNA and enzyme, they are not essential for enzyme function (see, e.g., Wang et al., J. Biol. Chem. 274:16508-12, 1999). Further, human and mouse cells express at least one QRS variant that contains a deletion in part 1 of the N-terminal region, possibly due to an alternate start codon or alternate splicing. However, the available sequence data for yeast suggests that these microorganisms do not express such a QRS variant, but rather only express a QRS polypeptide that contains both part I and part II of the N-terminal region.

Molecular phylogenetic studies of QRS suggest that it has relatively recently evolved from the closely related enzyme glutamyl-tRNA synthetase. As evidence, selected glutaminyl-tRNA synthetase mutants display enhanced glutamic acid recognition. For instance, mutagenesis of two residues proximal to the active site, Phe-90 and Tyr-240, improves glutamic acid recognition 3-5-fold in vitro and results in the misacylation of tRNA^{gln} with glutamic acid.

QRS has been crystallised in a variety of complexes, most importantly with its cognate tRNA^{gln}. The enzyme makes extensive contacts with the concave face of the tRNA, and makes specific interactions with the CUG anticodon at positions 34 to 36, and with the base pairs between the 5′ end and the 3′ end of the tRNA, just before the aminoacyl acceptor.

Certain QRS polypeptides possess anti-apoptotic activities. For instance, human QRS interacts with Fas ligation activated apoptosis signal-regulating kinase 1 (ASK1) in a glutamine-dependent manner. This interaction involves the catalytic domains of the two enzymes, and is dissociated by Fas ligand. This interaction also inhibits both ASK1 activity, as measured by in vitro kinase and transcription assays, and cell death induced by ASK1, an effect that is weakened by glutamine deprivation. The anti-apoptotic interaction of QRS with ASK1 is therefore enhanced by the cellular concentration of glutamine and reduced by Fas ligation. This anti-apoptotic activity is believed to lie in the C-terminal 539 amino acids of human QRS.

The amino acid sequence of the full-length QRS polypeptide is shown in SEQ ID NO:25. Certain specific examples of QRS variants, truncations, or fragments include QRS polypeptides that comprise or consist essentially of amino acids 1-183 (QRS1 or Q1), 1-220 (QRS2 or Q2), 1-249 (QRS3 or Q3), 1-200 (QRS4 or Q4), 1-(181-293), e.g., 1-180, 1-181, 1-182, 1-183, 1-184, 1-185, 1-186, 1-187, 1-188, 1-189, 1-190, 1-191, 1-192, 1-193, 1-194, 1-195, 1-196, 1-197, 1-198, 1-199, 1-200, etc., of SEQ ID NO:25 (see Table 2). Accordingly, these and other variants of QRS polypeptides are included within the AARS polypeptides of the present invention.

Glycyl-tRNA synthetase (GlyRS) is an α2 dimer that belongs to the class II family of tRNA synthetases (see, e.g., U.S. application Ser. No. 12/492,925, herein incorporated by reference). The approximately 2462 bp cDNA for this gene contains a large open reading frame (ORF) encoding 685 amino acids with predicted M(r)=77,507 Da. The protein sequence of human GlyRS has approximately 60% identity with B. mori GlyRS and 45% identity with S. cerevisiae GlyRS, and contains motifs 2 and 3 characteristic of Class II tRNA synthetases

The amino acid sequence of the full-length GlyRS polypeptide is shown in SEQ ID NO:16. SEQ ID NOS:18-24 represent illustrative peptide sequences analyzed in determining GlyRS fragment boundaries (see Example 9 & Table 1).

Certain examples of GlyRS proteolytic fragments include polypeptides that comprise, consist essentially of, or consist of amino acid residues 57-685, 214-685, 239-685, 311-685, 439-685, 511-658, 214-438, 367-438, 214-420, 214-338, 85-127 1-213, 1-61, 85-214, 333-685, 128-685, 265-685, 483-685 or 25-56 of SEQ ID NO:16 (see Table 1), including biologically active truncations or variants thereof (e.g., variants having about 80%, 85%, 90%, 95%, 98% sequence identity to the fragments) that substantially retain at least one non-canonical biological activity of interest. In certain specific embodiments, the GlyRS polypeptide is not a polypeptide as set forth in any one of NCBI # CR594947, U09587 and/or U09510. Accordingly, these and other variants of GlyRS polypeptides are included within the AARS polypeptides of the present invention.

Additional examples of AARS polypeptides having non-canonical activities include phenylalanyl-tRNA synthetase (PheRS) splice variant polypeptides (PheRS_SV1P) (SEQ ID NO:104), which have a unique amino acid sequence in the C-terminal end that is different from the full-length human PheRS protein sequence, including variants and fragments of those PheRS polypeptides; and aspartyl-tRNA synthetase (AspRS) polypeptides (SEQ ID NO:105), including fragments thereof that consist essentially of amino acid residues 1-154, 1-174, 1-31, 399-425, 413-476 or 397-425 of SEQ ID NO:105.

Embodiments of the present invention contemplate the use of compositions comprising hematopoiesis-modulating AARS polypeptides, including truncated fragments, splice variants, proteolytic fragments, and variants and/or modified polypeptides thereof, for modulating hematopoiesis in a subject. Variant proteins encompassed by the present application are biologically active, that is, they continue to possess the hematopoietic-modulating activity of a reference AARS polypeptide sequence (e.g., SEQ ID NOS: 1, 2, 3, 6, 8, 10, 12, 14, 16, 25, 28, 30, and 32-108). Such variants may result from, for example, genetic polymorphism or from human manipulation. Biologically active variants of a reference AARS polypeptide fragment will have at least 40%, 50%, 60%, 70%, generally at least 75%, 80%, 85%, usually about 90% to 95% or more, and typically about 98% or more sequence similarity or identity with the amino acid sequence for a reference protein as determined by sequence alignment programs described elsewhere herein using default parameters. A biologically active variant of a reference AARS polypeptide may differ from that protein generally by as much 200, 100, 50 or 20 amino acid residues or suitably by as few as 1-15 amino acid residues, as few as 1-10, such as 6-10, as few as 5, as few as 4, 3, 2, or even 1 amino acid residue. In some embodiments, an AARS polypeptide differs from the reference sequences in SEQ ID NOS: 1, 2, 3, 6, 8, 10, 12, 14, 16, 25, 28, 30, or 32-108 by at least one but by less than 15, 10 or 5 amino acid residues. In other embodiments, it differs from the reference sequences in SEQ ID NOS: 1, 2, 3, 6, 8, 10, 12, 14, 16, 25, 28, 30, or 32-108 by at least one residue but less than 20%, 15%, 10% or 5% of the residues.

An AARS polypeptide may be altered in various ways including amino acid substitutions, deletions, truncations, and insertions. Methods for such manipulations are generally known in the art. For example, amino acid sequence variants of a truncated and/or variant AARS polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of AARS polypeptides. Recursive ensemble mutagenesis (REM), a technique which enhances the frequency of functional mutants in the libraries, can be used in combination with the screening assays to identify AARS polypeptide variants (Arkin and Yourvan (1992) Proc. Natl. Acad. Sci. USA 89: 7811-7815; Delgrave et al., (1993) Protein Engineering, 6: 327-331). Conservative substitutions, such as exchanging one amino acid with another having similar properties, may be desirable as discussed in more detail below.

Biologically active truncated and/or variant AARS polypeptides may contain conservative amino acid substitutions at various locations along their sequence, as compared to a reference AARS amino acid sequence (e.g., SEQ ID NOS: 1, 2, 3, 6, 8, 10, 12, 14, 16, 25, 28, 30, and 32-108). A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices are known in the art (see e.g., PAM120 matrix and PAM250 matrix as disclosed for example by Dayhoff et al., 1978, A model of evolutionary change in proteins). Matrices for determining distance relationships In M. O. Dayhoff, (ed.), Atlas of protein sequence and structure, Vol. 5, pp. 345-358, National Biomedical Research Foundation, Washington D.C.; and by Gonnet et al., (Science, 256: 14430-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table A.

TABLE A
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence | |
| chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional truncated and/or variant AARS polypeptide can readily be determined by assaying its activity, as described herein (see, e.g., Examples 1, 2, 10, and 11). Conservative substitutions are shown in Table B under the heading of exemplary substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, (c) the bulk of the side chain, or (d) the biological function. After the substitutions are introduced, the variants are screened for biological activity.

TABLE B
Exemplary Amino Acid Substitutions

| Original | | Preferred |
|---|---|---|
| Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

Thus, a predicted non-essential amino acid residue in a truncated and/or variant AARS polypeptide is typically replaced with another amino acid residue from the same side chain family. Alternatively, mutations can be introduced randomly along all or part of an AARS coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined. A “non-essential” amino acid residue is a residue that can be altered from the reference sequence of an embodiment polypeptide without abolishing or substantially altering one or more of its activities. Suitably, the alteration does not substantially abolish one of these activities, for example, the activity is at least 20%, 40%, 60%, 70% or 80% 100%, 500%, 1000% or more of the reference AARS sequence. An “essential” amino acid residue is a residue that, when altered from the reference sequence of an AARS polypeptide, results in abolition of an activity of the parent molecule such that less than 20% of the reference activity is present. For example, such essential amino acid residues include those that are conserved in AARS polypeptides across different species, including those sequences that are conserved in the active binding site(s) or motif(s) of AARS polypeptides from various sources.

Accordingly, the present invention also contemplates variants of the naturally-occurring AARS polypeptide sequences or their biologically-active fragments, wherein the variants are distinguished from the naturally-occurring sequence by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity or sequence identity to a reference AARS polypeptide sequence, for example, as set forth in SEQ ID NOS: 1, 2, 3, 6, 8, 10, 12, 14, 16, 25, 28, 30, and 32-108. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids but which retain the properties of a parent or reference AARS polypeptide sequence are contemplated. In certain embodiments, the C-terminal or N-terminal region of any AARS polypeptide, including the AARS polypeptides of SEQ ID NOS: 1, 2, 3, 6, 8, 10, 12, 14, 16, 25, 28, 30, or 32-108, may be truncated by about 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 250, 300, 350, 400, 450, 500, 550, 600, 650, or 700 or more amino acids, or by about 10-50, 20-50, 50-100, 100-150, 150-200, 200-250, 250-300, 300-350, 350-400, 400-450, 450-500, 500-550, 550-600, 600-650, 650-700 or more amino acids, including all integers and ranges in between (e.g., 101, 102, 103, 104, 105), so long as the truncated AARS polypeptide is capable of modulating hematopoiesis, either in vivo, in vitro, or ex vivo (e.g., stimulating megakaryocyte progenitor accumulation, stimulating neutrophil proliferation, reducing erythroid progenitor cell formation).

In some embodiments, variant polypeptides differ from a reference AARS sequence by at least one but by less than 50, 40, 30, 20, 15, 10, 8, 6, 5, 4, 3 or 2 amino acid residue(s). In other embodiments, variant polypeptides differ from the corresponding sequences of SEQ ID NOS: 1, 2, 3, 6, 8, 10, 12, 14, 16, 25, 28, 30, or 32-108 by at least 1% but less than 20%, 15%, 10% or 5% of the residues. (If this comparison requires alignment, the sequences should be aligned for maximum similarity. “Looped” out sequences from deletions or insertions, or mismatches, are considered differences.) The differences are, suitably, differences or changes at a non-essential residue or a conservative substitution.

In certain embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more sequence identity or similarity to a corresponding sequence of an AARS polypeptide as, for example, set forth in SEQ ID NOS: 1, 2, 3, 6, 8, 10, 12, 14, 16, 25, 28, 30, or 32-108 and has the ability to modulate hematopoiesis in a subject, such as by reducing the proliferation of progenitor cells of the erythroid lineage, stimulating the proliferation and/or differentiation of megakaryocytes in a subject, and/or stimulating the proliferation of neutrophils in a subject.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

Variants of an AARS polypeptide can be identified by screening combinatorial libraries of mutants of an AARS polypeptide. Libraries or fragments e.g., N terminal, C terminal, or internal fragments, of AARS protein coding sequence can be used to generate a variegated population of fragments for screening and subsequent selection of variants of an AARS polypeptide.

Methods for screening gene products of combinatorial libraries made by point mutation or truncation, and for screening cDNA libraries for gene products having a selected property are known in the art. Such methods are adaptable for rapid screening of the gene libraries generated by combinatorial mutagenesis of AARS polypeptides.

Also included are proteolytic fragments of AARS polypeptides. In certain illustrative embodiments, proteolytic fragments of AARS polypeptides may be produced using a variety of proteolytic enzymes or proteolytic chemical agents, according to techniques known and available in the art. Proteolytic fragments can be produced in vitro, such as by incubating AARS polypeptides with one or more proteases (as described herein and known in the art) under controlled conditions and isolating and characterizing the fragments produced therefrom. Proteolytic fragments can also be produced in vivo, or endogenously, such as by recombinantly expressing the AARS polypeptides in a selected cell (e.g., bacterial cell, eukaryotic cell), and isolating and characterizing the endogenous fragments produced therefrom (see, e.g., Example 10).

Proteases are usually classified according to three major criteria: (i) the reaction catalysed, (ii) the chemical nature of the catalytic site, and (iii) the evolutionary relationship, as revealed by the structure. General examples of proteases or proteinases, as classified by mechanism of catalysis, include aspartic proteases, serine proteases, cysteine proteases, and metalloproteases.

Most aspartic proteases belong to the pepsin family. This family includes digestive enzymes, such as pepsin and chymosin, as well as lysosomal cathepsins D and processing enzymes such as renin, and certain fungal proteases (e.g., penicillopepsin, rhizopuspepsin, endothiapepsin). A second family of aspartic proteases includes viral proteinases such as the protease from the AIDS virus (HIV), also called retropepsin.

Serine proteases include two distinct families. First, the chymotrypsin family, which includes the mammalian enzymes such as chymotrypsin, trypsin, elastase, and kallikrein, and second, the substilisin family, which includes the bacterial enzymes such as subtilisin. The general 3D structure between these two families is different, but they have the same active site geometry, and catalysis proceeds via the same mechanism. The serine proteases exhibit different substrate specificities, differences which relate mainly to amino acid substitutions in the various enzyme subsites (substrate residue interacting sites). Some serine proteases have an extended interaction site with the substrate whereas others have a specificity that is restricted to the P1 substrate residue.

The cysteine protease family includes the plant proteases such as papain, actinidin, and bromelain, several mammalian lysosomal cathepsins, the cytosolic calpains (calcium-activated), as well as several parasitic proteases (e.g., Trypanosoma, Schistosoma). Papain is the archetype and the best studied member of the family. Recent elucidation of the X-ray structure of the Interleukin-1-beta Converting Enzyme has revealed a novel type of fold for cysteine proteinases.

The metalloproteases are one of the older classes of proteases, found in bacteria, fungi, and higher organisms. They differ widely in their sequences and their 3D structures, but the great majority of enzymes contain a zinc atom that is catalytically active. In some cases, zinc may be replaced by another metal such as cobalt or nickel without loss of proteolytic activity. Bacterial thermolysin has been well characterized and its crystallographic structure indicates that zinc is bound by two histidines and one glutamic acid. Many metalloproteases contain the sequence motif HEXXH, which provides two histidine ligands for the zinc. The third ligand is either a glutamic acid (thermolysin, neprilysin, alanyl aminopeptidase) or a histidine (astacin, serralysin).

Illustrative proteases include, for example, achromopeptidase, aminopeptidase, ancrod, angiotensin converting enzyme, bromelain, calpain, calpain I, calpain II, carboxypeptidase A, carboxypeptidase B, carboxypeptidase G, carboxypeptidase P, carboxypeptidase W, carboxypeptidase Y, caspase 1, caspase 2, caspase 3, caspase 4, caspase 5, caspase 6, caspase 7, caspase 8, caspase 9, caspase 10, caspase 11, caspase 12, caspase 13, cathepsin B, cathepsin C, cathepsin D, cathepsin E, cathepsin G, cathepsin H, cathepsin L, chymopapain, chymase, chymotrypsin, clostripain, collagenase, complement C1r, complement C1 s, complement Factor D, complement factor I, cucumisin, dipeptidyl peptidase IV, elastase (leukocyte), elastase (pancreatic), endoproteinase Arg-C, endoproteinase Asp-N, endoproteinase Glu-C, endoproteinase Lys-C, enterokinase, factor Xa, ficin, furin, granzyme A, granzyme B, HIV Protease, IGase, kallikrein tissue, leucine aminopeptidase (general), leucine aminopeptidase (cytosol), leucine aminopeptidase (microsomal), matrix metalloprotease, methionine aminopeptidase, neutrase, papain, pepsin, plasmin, prolidase, pronase E, prostate specific antigen, protease alkalophilic from Streptomyces griseus, protease from Aspergillus, protease from Aspergillus saitoi, protease from Aspergillus sojae, protease (B. licheniformis) (alkaline or alcalase), protease from Bacillus polymyxa, protease from Bacillus sp, protease from Rhizopus sp., protease S, proteasomes, proteinase from Aspergillus oryzae, proteinase 3, proteinase A, proteinase K, protein C, pyroglutamate aminopeptidase, rennin, rennin, streptokinase, subtilisin, thermolysin, thrombin, tissue plasminogen activator, trypsin, tryptase and urokinase.

Tables C-G illustrate the type of proteolytic fragments that can be produced in vitro by incubating AARS polypeptides with various proteases. In certain embodiments, the incubation conditions can be controlled so that only certain cleavage sites are cleaved by the indicated protease, to achieve only partial cleavage, followed by isolation of the desired proteolytic fragment according to techniques known in the art (e.g., chromatography). Once a desired fragment has been isolated and characterized (e.g., sequenced) according to routine techniques in the art, it can be cloned and produced recombinantly, or produced synthetically, as desired.

Hence, included within the AARS polypeptides of the invention are any proteolytic fragments that can be produced by the exemplary proteases in Tables C-G, in addition to the proteases listed elsewhere herein, including any combination of proteases (e.g., Caspase 1 and hydroxylamine), or any combination of individual cleavage sites. Also, the residue position of cleavage sites may be approximate. Merely by way of illustration, an AARS proteolytic fragment may include about residues 1-165, about residues 166-445, about residues 166-455, about residues 166-716, about residues 445-716, or about residues 455-716 of GlyRS that has been cleaved or partially cleaved by incubation with iodosobenzoic acid (see Table C). As an additional illustration, an AARS proteolytic fragment may include about residues 1-98, about residues 1-135, about residues 98-135, about residues 1-234, about residues 98-234, about residues 1-379, about residues 234-674, or about residues 135-737 of QRS that has been cleaved or partially cleaved by proline-endopeptidase (see Table D). As a further illustrative example, an AARS polypeptide may include about residues 1-210, about residues 1-273, about residues 1-295, about residues 210-273, about residues 210-295, about residues 273-295 of QRS that has been cleaved or partially cleaved by hydroxylamine. Similar patterns can be applied to any of the AARS polypeptides and any of the proteases in Tables C-G, or to the other proteases listed herein or known in the art.

TABLE C
Glycyl-tRNA synthetase (EC 6.1.1.14) (Glycine-tRNA ligase) (GlyRS)

| Protease | Position of cleavage sites (Residue No.) |
|---|---|
| Arg-C proteinase | 5 10 13 23 27 33 34 52 68 72 79 101 103 121 130 131 166 213 297 310 331 337 |
| | 342 344 388 391 412 428 430 464 474 560 583 596 602 640 656 657 660 687 689 |
| | 693 696 722 |
| Asp-N endopeptidase | 55 75 83 89 91 115 116 119 125 134 148 178 195 199 204 214 227 246 255 270 |
| | 355 360 369 393 424 442 445 462 467 510 522 553 598 647 648 661 672 674 687 |
| | 689 707 717 |
| Asp-N endopeptidase + | 55 60 61 75 82 83 89 91 96 105 108 115 116 119 124 125 134 148 171 172 176 |
| N-terminal Glu | 178 185 195 199 204 209 214 227 233 237 239 246 252 255 270 298 312 332 344 |
| | 349 351 355 358 360 369 393 396 424 433 435 442 445 447 456 462 467 482 487 |
| | 497 510 516 522 523 528 530 535 538 542 544 553 567 568 575 589 596 598 599 |
| | 625 632 635 647 648 661 662 672 674 687 689 697 700 707 717 719 726 729 734 |
| | 737 738 |
| BNPS-Skatole | 165 445 455 716 |
| CNBr | 1 55 124 182 202 226 239 281 292 348 390 433 437 516 530 532 555 585 628 |
| | 692 |
| Caspase 1 | 215 |
| Chymotrypsin high | 132 133 134 138 141 148 150 165 169 198 201 212 249 258 261 278 282 285 295 |
| specificity (C term to | 305 308 314 321 330 346 354 365 374 376 408 409 414 416 429 440 445 453 455 |
| [FYW], not before P) | 467 497 508 518 526 540 549 561 566 579 586 589 593 604 605 614 627 630 658 |
| | 668 674 716 726 |
| Clostripain | 5 10 13 23 27 33 34 52 68 72 79 101 103 121 130 131 166 213 297 310 331 337 |
| | 342 344 388 391 412 428 430 464 474 560 583 596 602 640 656 657 660 687 689 |
| | 693 696 722 |
| Formic acid | 56 76 84 90 92 116 117 120 126 135 149 179 196 200 205 215 228 247 256 271 |
| | 356 361 370 394 425 443 446 463 468 511 523 554 599 648 649 662 673 675 688 |
| | 690 708 718 |
| Glutamyl endopeptidase | 61 62 83 97 106 109 125 172 173 177 186 210 234 238 240 253 299 313 333 345 |
| | 350 352 359 397 434 436 448 457 483 488 498 517 524 529 531 536 539 543 545 |
| | 568 569 576 590 597 600 626 633 636 663 698 701 720 727 730 735 738 739 |
| Hydroxylamine | 208 711 |
| Iodosobenzoic acid | 165 445 455 716 |
| LysC | 80 82 85 93 99 102 108 115 123 129 158 190 197 204 207 219 224 229 230 235 |
| | 236 264 283 309 318 360 364 379 389 419 426 450 477 484 487 490 501 506 509 |
| | 510 513 537 547 553 559 563 615 632 646 679 733 734 |
| LysN | 79 81 84 92 98 101 107 114 122 128 157 189 196 203 206 218 223 228 229 234 |
| | 235 263 282 308 317 359 363 378 388 418 425 449 476 483 486 489 500 505 508 |
| | 509 512 536 546 552 558 562 614 631 645 678 732 733 |
| NTCB (2-nitro-5- | 40 154 179 210 230 441 443 460 465 470 521 524 615 |
| thiocyanobenzoic acid) | |
| Proline-endopeptidase | 6 28 298 363 485 |
| Staphylococcal peptidase I | 61 83 97 106 109 125 172 177 186 210 234 238 240 253 299 313 333 345 350 |
| | 352 359 397 434 436 448 457 483 488 498 517 524 529 531 536 539 543 545 568 |
| | 576 590 597 600 626 633 636 663 698 701 720 727 730 735 738 |
| Trypsin | 10 13 23 33 34 52 68 72 79 80 82 85 93 99 101 102 103 108 115 121 123 129 130 |
| | 131 158 166 190 197 204 207 213 219 224 229 230 235 236 264 283 309 310 318 |
| | 331 337 342 344 360 364 379 388 389 391 412 419 426 428 430 450 464 474 477 |
| | 487 490 501 506 509 510 513 537 547 553 559 560 563 583 596 602 615 632 640 |
| | 646 656 657 660 679 687 689 693 696 722 733 734 |

TABLE D
Glutaminyl-tRNA synthetase (EC 6.1.1.18) (Glutamine-tRNA ligase) (QRS)

| Protease | Positions of cleavage sites (Residue No.) |
|---|---|
| Arg-C proteinase | 21 34 62 64 67 68 95 109 132 134 141 154 195 201 202 225 265 267 301 351 352 |
| | 361 376 378 391 403 419 427 463 464 486 497 509 515 523 524 525 538 558 567 |
| | 576 616 629 639 666 667 690 694 745 764 |
| Asp-N endopeptidase | 4 48 64 99 102 105 160 169 183 199 205 214 302 303 319 336 339 376 409 413 |
| | 429 438 445 474 509 511 512 558 562 588 597 617 668 702 713 723 728 738 751 |
| | 753 770 |
| Asp-N endopeptidase + | 4 16 21 34 48 64 83 91 99 102 105 107 109 119 122 123 126 139 151 160 167 |
| N-terminal Glu | 169 181 183 185 196 197 199 205 208 211 214 221 226 235 257 270 302 303 307 |
| | 309 310 319 336 339 347 362 363 376 380 381 387 396 398 408 409 413 429 438 |
| | 445 448 458 474 482 509 511 512 529 548 553 558 562 572 588 597 598 614 617 |
| | 620 621 623 645 658 661 668 671 687 692 701 702 705 713 723 728 738 743 751 |
| | 753 769 770 |
| BNPS-Skatole | 159 324 345 375 432 469 482 511 632 680 |
| CNBr | 1 146 150 164 171 221 250 321 380 390 404 408 413 548 569 686 |
| Caspase1 | 184 |
| Chymotrypsin-high | 10 57 71 75 93 107 142 144 159 189 231 238 243 286 288 290 299 302 314 315 |
| specificity (C-term to | 324 327 330 334 338 339 343 345 356 375 387 395 418 422 432 438 440 460 467 |
| [FYW], not before P) | 468 469 477 482 484 491 511 517 535 603 608 613 619 627 632 643 677 680 692 |
| | 696 711 738 741 743 748 749 762 |
| Clostripain | 21 34 62 64 67 68 95 109 132 134 141 154 195 201 202 225 265 267 301 351 352 |
| | 361 376 378 391 403 419 427 463 464 486 497 509 515 523 524 525 538 558 567 |
| | 576 616 629 639 666 667 690 694 745 764 |
| Formic acid | 5 49 65 100 103 106 161 170 184 200 206 215 303 304 320 337 340 377 410 414 |
| | 430 439 446 475 510 512 513 559 563 589 598 618 669 703 714 724 729 739 752 |
| | 754 771 |
| Glutamyl endopeptidase | 17 22 35 84 92 108 110 120 123 124 127 140 152 168 182 186 197 198 209 212 |
| | 222 227 236 258 271 308 310 311 348 363 364 381 382 388 397 399 409 449 459 |
| | 483 530 549 554 573 599 615 621 622 624 646 659 662 672 688 693 702 706 744 |
| | 770 |
| Hydroxylamine | 210 273 295 |
| Iodosobenzoic acid | 159 324 345 375 432 469 482 511 632 680 |
| LysC | 19 25 50 79 80 158 163 166 180 187 188 190 193 205 230 233 239 254 282 292 |
| | 309 313 331 366 392 394 405 412 421 431 458 496 498 586 601 620 628 652 673 |
| | 675 699 736 740 759 769 774 |
| LysN | 18 24 49 78 79 157 162 165 179 186 187 189 192 204 229 232 238 253 281 291 |
| | 308 312 330 365 391 393 404 411 420 430 457 495 497 585 600 619 627 651 672 |
| | 674 698 735 739 758 768 773 |
| NTCB (2-nitro-5- | 110 297 318 357 432 442 444 455 470 477 535 555 656 664 686 729 |
| thiocyanobenzoic acid) | |
| Proline-endopeptidase | 98 135 234 379 674 737 |
| Staphylococcal peptidase I | 17 22 35 84 92 108 110 120 123 127 140 152 168 182 186 197 209 212 222 227 |
| | 236 258 271 308 310 348 363 381 388 397 399 409 449 459 483 530 549 554 573 |
| | 599 615 621 624 646 659 662 672 688 693 702 706 744 770 |
| Thrombin | 567 |
| Trypsin | 19 21 25 34 50 62 64 67 68 79 80 95 109 132 141 154 158 163 166 180 187 188 |
| | 190 193 195 201 202 205 225 230 239 254 265 267 282 292 301 309 313 331 351 |
| | 352 361 366 376 391 392 394 403 405 412 419 421 427 431 458 463 464 486 496 |
| | 497 498 509 515 523 525 538 558 567 576 586 601 616 620 628 629 639 652 666 |
| | 667 675 690 694 699 740 745 759 764 769 774 |

TABLE E
Tryptophanyl-tRNA synthetase, cytoplasmic (EC 6.1.1.2) (Tryptophan-
tRNA ligase) (WRS) (Interferon-induced protein 53) (IFP53) (hWRS)

| Protease | Positions of cleavage sites (Residue No.) |
|---|---|
| Arg-C proteinase | 24 106 119 122 127 133 134 141 162 298 300 318 321 326 381 388 417 448 |
| | 449 464 |
| Asp-N endopeptidase | 33 36 56 60 75 82 85 98 100 112 141 147 184 196 197 204 208 220 227 236 |
| | 238 270 272 298 301 311 313 321 353 362 381 394 396 408 409 410 418 453 |
| | 468 |
| Asp-N endopeptidase + N- | 4 10 20 33 34 36 55 56 60 75 78 80 81 82 85 98 100 112 114 120 141 147 |
| terminal Glu | 150 166 184 196 197 198 204 208 216 220 227 236 238 270 272 298 301 311 |
| | 313 321 353 362 381 384 385 394 396 407 408 409 410 413 418 428 435 443 |
| | 450 453 454 458 468 |
| BNPS-Skatole | 88 182 203 |
| CNBr | 1 42 48 143 169 195 241 243 319 350 401 425 461 |
| Caspase1 | 61 363 |
| Chymotrypsin-high specificity | 13 50 58 84 88 100 107 131 137 138 150 156 157 159 177 179 182 187 201 |
| (C-term to [FYW], not before | 203 212 214 227 233 235 240 247 248 260 267 269 289 297 316 317 339 360 |
| P) | 377 390 400 402 405 406 420 460 468 470 |
| Clostripain | 24 106 119 122 127 133 134 141 162 298 300 318 321 326 381 388 417 448 |
| | 449 464 |
| Enterokinase | 200 412 |
| Formic acid | 34 37 57 61 76 83 86 99 101 113 142 148 185 197 198 205 209 221 228 237 |
| | 239 271 273 299 302 312 314 322 354 363 382 395 397 409 410 411 419 454 |
| | 469 |
| Glutamyl endopeptidase | 5 11 21 35 56 79 81 82 115 121 151 167 199 217 385 386 408 414 429 436 |
| | 444 451 455 459 |
| Iodosobenzoic acid | 88 182 203 |
| LysC | 27 33 41 47 51 59 96 102 111 114 153 154 181 200 204 220 231 249 253 256 |
| | 264 277 331 349 366 369 371 374 412 418 431 432 450 458 465 |
| LysN | 26 32 40 46 50 58 95 101 110 113 152 153 180 199 203 219 230 248 252 255 |
| | 263 276 330 348 365 368 370 373 411 417 430 431 449 457 464 |
| NTCB (2-nitro-5- | 61 224 273 304 308 393 |
| thiocyanobenzoic acid) | |
| Proline-endopeptidase | 128 155 332 |
| Staphylococcal peptidase I | 5 11 21 35 56 79 81 115 121 151 167 199 217 385 408 414 429 436 444 451 |
| | 455 459 |
| Thrombin | 162 326 |
| Trypsin | 24 27 33 41 47 51 59 96 102 106 111 114 119 122 133 134 141 153 162 181 |
| | 200 204 220 231 249 253 256 264 277 298 300 318 321 326 349 366 369 371 |
| | 374 381 388 412 417 418 431 432 448 449 450 458 464 465 |

TABLE F
Tyrosyl-tRNA synthetase (EC 6.1.1.1) (Tyrosyl-tRNA ligase) (YRS)

| Protease | Positions of cleavage sites (Residue No.) |
|---|---|
| Arg-C proteinase | 16 34 93 135 189 207 237 279 325 367 371 400 418 432 450 |
| Asp-N endopeptidase | 2 60 74 80 121 131 143 172 179 186 232 235 239 279 293 297 307 321 342 368 |
| | 382 384 392 416 455 477 493 |
| Asp-N endopeptidase + N- | 2 7 8 19 23 24 28 32 34 60 67 74 80 87 90 97 105 112 121 127 131 143 150 156 |
| terminal Glu | 172 173 174 179 186 195 226 227 228 232 235 238 239 250 255 273 279 280 |
| | 293 295 297 301 307 313 321 325 342 358 360 361 368 378 382 384 389 392 |
| | 395 397 412 413 416 434 445 452 455 464 472 477 478 479 488 493 498 499 |
| BNPS-Skatole | 40 87 283 505 |
| CNBr | 1 56 83 104 211 214 223 350 431 439 511 |
| Caspase1 | 75 494 |
| Chymotrypsin-high | 39 40 52 53 62 73 79 87 96 97 117 123 129 134 176 183 192 194 198 204 249 |
| specificity (C-term to | 263 275 283 289 292 299 328 388 409 468 472 488 495 505 510 |
| [FYW], not before P) | |
| Clostripain | 16 34 93 135 189 207 237 279 325 367 371 400 418 432 450 |
| Formic acid | 3 61 75 81 122 132 144 173 180 187 233 236 240 280 294 298 308 322 343 369 |
| | 383 385 393 417 456 478 494 |
| Glutamyl endopeptidase | 8 9 20 24 25 29 33 35 68 88 91 98 106 113 128 151 157 174 175 196 227 228 |
| | 229 239 251 256 274 281 296 302 314 326 359 361 362 379 390 396 398 413 |
| | 414 435 446 453 465 473 479 480 489 499 500 |
| Hydroxylamine | 258 |
| Iodosobenzoic acid | 40 87 283 505 |
| LysC | 10 26 28 32 37 47 58 64 84 102 114 116 119 127 146 147 154 178 190 197 206 |
| | 222 231 238 242 243 244 246 247 265 272 282 287 297 310 319 327 334 335 |
| | 346 348 352 356 374 380 391 412 427 430 470 474 482 484 485 486 490 496 |
| | 506 513 520 523 |
| LysN | 9 25 27 31 36 46 57 63 83 101 113 115 118 126 145 146 153 177 189 196 205 |
| | 221 230 237 241 242 243 245 246 264 271 281 286 296 309 318 326 333 334 |
| | 345 347 351 355 373 379 390 411 426 429 469 473 481 483 484 485 489 495 |
| | 505 512 519 522 |
| NTCB (2-nitro-5- | 66 249 423 441 500 518 |
| thiocyanobenzoic acid) | |
| Proline-endopeptidase | 48 159 306 349 382 428 483 |
| Staphylococcal peptidase I | 8 20 24 29 33 35 68 88 91 98 106 113 128 151 157 174 196 227 239 251 256 |
| | 274 281 296 302 314 326 359 361 379 390 396 398 413 435 446 453 465 473 |
| | 479 489 499 |
| Trypsin | 10 16 26 28 32 34 37 58 64 84 93 102 114 116 119 127 135 146 147 154 178 |
| | 189 190 197 206 207 222 231 237 238 242 243 244 246 247 265 272 279 282 |
| | 287 297 310 319 325 327 334 335 346 352 356 367 371 374 380 391 400 412 |
| | 418 430 432 450 470 474 484 485 486 490 496 506 513 520 523 |

TABLE G
Histidyl-tRNA synthetase (EC 6.1.1.21) (Histidine-tRNA ligase) (HisRS)

| Protease | Positions of cleavage sites (Residue No.) |
|---|---|
| Arg-C proteinase | 4 17 19 63 68 73 82 86 128 137 149 157 158 165 167 169 214 215 232 266 326 |
| | 362 375 388 396 405 424 479 484 490 491 500 501 |
| Asp-N endopeptidase | 47 63 77 92 109 115 118 129 158 174 176 182 187 205 212 217 227 238 241 |
| | 264 268 285 300 314 315 320 328 363 370 432 472 487 492 |
| Asp-N endopeptidase + N- | 2 7 8 15 28 31 32 33 47 48 63 73 77 89 92 97 100 108 109 115 118 122 129 158 |
| terminal Glu | 169 174 176 182 187 189 196 205 212 217 227 238 241 246 247 251 255 261 |
| | 264 268 280 285 296 300 306 314 315 320 328 336 348 349 363 370 386 393 |
| | 397 400 401 407 421 422 429 432 438 455 456 467 469 472 484 485 487 491 |
| | 492 495 496 |
| BNPS-Skatole | 246 432 |
| CNBr | 1 70 104 141 163 185 195 220 253 369 |
| Chymotrypsin high | 54 65 77 84 97 107 115 129 135 138 150 156 168 171 172 176 182 207 221 231 |
| specificity (C term to | 246 270 306 308 312 320 330 331 336 363 370 390 432 442 454 |
| [FYW], not before P) | |
| Clostripain | 4 17 19 63 68 73 82 86 128 137 149 157 158 165 167 169 214 215 232 266 326 |
| | 362 375 388 396 405 424 479 484 490 491 500 501 |
| Formic acid | 48 64 78 93 110 116 119 130 159 175 177 183 188 206 213 218 228 239 242 |
| | 265 269 286 301 315 316 321 329 364 371 433 473 488 493 |
| Glutamyl endopeptidase | 3 8 9 16 29 32 33 34 49 74 90 98 101 109 123 170 190 197 247 248 252 256 |
| | 262 281 297 307 337 349 350 387 394 398 401 402 408 422 423 430 439 456 |
| | 457 468 470 485 486 492 496 497 |
| Iodosobenzoic acid | 246 432 |
| LysC | 12 22 25 37 40 42 51 53 57 60 75 85 100 106 112 118 143 148 154 193 210 230 |
| | 240 243 250 257 288 293 303 317 373 376 403 418 419 426 437 443 444 447 |
| | 472 477 499 |
| LysN | 11 21 24 36 39 41 50 52 56 59 74 84 99 105 111 117 142 147 153 192 209 229 |
| | 239 242 249 256 287 292 302 316 372 375 402 417 418 425 436 442 443 446 |
| | 471 476 498 |
| NTCB (2-nitro-5- | 82 173 190 195 223 234 378 454 506 508 |
| thiocyanobenzoic acid) | |
| Staphylococcal peptidase I | 3 8 16 29 32 49 74 90 98 101 109 123 170 190 197 247 252 256 262 281 297 |
| | 307 337 349 387 394 398 401 408 422 430 439 456 468 470 485 492 496 |
| Trypsin | 4 12 17 19 22 25 37 40 42 51 53 57 60 63 68 73 75 82 85 86 100 106 112 118 |
| | 128 137 143 148 149 154 157 158 165 167 169 193 210 214 215 230 232 240 |
| | 243 250 257 266 288 293 303 317 326 362 373 375 376 388 396 403 405 418 |
| | 419 424 426 437 443 444 447 472 477 479 484 490 491 499 500 501 |

Certain embodiments relate to isolated AARS polypeptides, comprising, consisting essentially of, or consisting of amino acid sequences that have been derived from endogenous, naturally-occurring AARS polypeptide fragments, and pharmaceutical compositions comprising said fragments, and methods of use thereof. In certain embodiments, as noted above, the sequences of naturally-occurring endogenous proteolytic fragments can be generated or identified, for instance, from various cellular fractions (e.g., cytosolic, membrane, nuclear) and/or conditioned medium from various cell-types, including primary cells and cell lines. Examples of such cell types include, without limitation, immune cells such as dendritic cells, monocytes/macrophages (e.g., RAW 264.7 macrophages; see Example 5), neutrophils, eosinophils, basophils, and lymphocytes, such as B-cells and T-cells (e.g., CD4+ helper and CD8+ killer cells), including primary T-cells and T-cell lines such as Jurkat T-cells, as well as natural killer (NK) cells.

In certain embodiments, endogenous proteolytic fragments can be identified by techniques such as mass-spectrometry, or equivalent techniques. Merely by way of illustration and not limitation, in certain embodiments the proteomes from various cell types or fractions thereof may be separated by 1D SDS-PAGE and the gel lanes cut into bands at fixed intervals; after which the bands may be optionally digested with an appropriate protease, such as trypsin, to release the peptides, which may then be analyzed by 1D reverse phase LC-MS/MS. The resulting proteomic data may be integrated into so-called peptographs, which plot, in the left panel, sequence coverage for a given protein in the horizontal dimension (N to C terminus, left to right) versus SDS-PAGE migration in the vertical dimension (high to low molecular weight, top to bottom). The specific peptide fragments can then be sequenced or mapped. Table H provides a set of illustrative mouse QRS polypeptide fragments that were identified from RAW macrophages according to these exemplary techniques. Table I provides the corresponding set of human QRS polypeptide fragments.

TABLE H
Mouse QRS Polypeptide Fragments

| PEPTIDE SEQUENCE | SEQ ID NO: |
|---|---|
| ETLKNEALSTQLR | 36 |
| EAATQAHQILGSTIDKATGVLLYDLVSR | 37 |
| ETLKNEALSTQLREAATQAHQILGSTIDKATGVLLYDLVSR | 38 |
| DFEQECGVGVVVTPEQIEEAVESTINK | 39 |
| FNMGLLMGEAR* | 40 |
| MIKNEVDMQVLHLLGPK* | 41 |
| NEVDMQVLHLLGPK* | 42 |
| TPGYVITPYTMDLLK | 43 |
| FDDTNPEKEEAK* | 44 |
| VEELKGHNPLPSPWR | 45 |
| DRPKEESLLLFEAMR | 46 |
| VEELKGHNPLPSPWRDRPKEESLLLFEAMR | 47 |
| LVMEDGKMDPVAYR* | 48 |
| VYCPVQWEYGR* | 49 |
| ILQLVAAGAVR | 50 |
| DVLNDAAPRAMAVLEPLQVVITNFPAPK | 51 |
| GFHQVPFASTVFIERSDFKEESEPGYKRLASGQPVGLR | 52 |
| AFIHWVSQPLVCEIR | 53 |
| LGYFSVDPDSHQGQIVFNR | 54 |
| TPGYVITPYTMDLLK | 55 |
| AINFNFGYAK* | 56 |
| FDDTNPEKEEAK* | 57 |
| FFTAIYDMVTWLGYTPYK | 58 |
| FDDTNPEKEEAKFFTAIYDMVTWLGYTPYK | 59 |
| DRPKEESLLLFEAMR | 60 |
| VYCPVQWEYGR* | 61 |
| LNLHYAVVSK* | 62 |
| VYCPVQWEYGRLNLHYAVVSK* | 63 |
| ILQLVAAGAVR | 64 |
| AMAVLEPLQVVITNFPAPK | 65 |
| PLDIRVPNFPADETK | 66 |
| AMAVLEPLQVVITNFPAPKPLDIRVPNFPADETK | 67 |
| SDFKEESEPGYKRLASGQPVGLRHTGYVIELQNIVR | 68 |
| AFIHWVSQPLVCEIR | 69 |
| LGYFSVDPDSHQGQIVFNR | 70 |
| KATGVLLYDLVSR | 71 |
| SFLVSYIANK | 72 |
| DFEQECGVGVVVTPEQIEEAVESTINK | 73 |
| MIKNEVDMQVLHLLGPK* | 74 |
| EAATQAHQILGSTIDKATGVLLYDLVSR | 75 |

*The mouse and human sequences are identical.

TABLE I
Human QRS Polypeptide Fragments

| PEPTIDE SEQUENCE | SEQ ID NO: |
|---|---|
| ETLKNSALSAQLR | 76 |
| EAATQAQQTLGSTIDKATGILLYGLASR | 77 |
| ETLKNSALSAQLREAATQAQQTLGSTIDKATGILLYGLASR | 78 |
| DFERECGVGVIVTPEQIEEAVEAAINR | 79 |
| TPGYVVTPHTMNLLK | 80 |
| GEELKGHNTLPSPWR | 81 |
| DRPMEESLLLFEAMR | 82 |
| GEELKGHNTLPSPWRDRPMEESLLLFEAMR | 83 |
| ILQLVATGAVR | 84 |
| DVLNDTAPRAMAVLESLRVIITNFPAAK | 85 |
| GFHQVPFAPIVFIERTDFKEEPEPGFKRLAWGQPVGLR | 86 |
| AFIHWVSQPLMCEVR | 87 |
| LGYFSVDPDSHQGKLVFNR | 88 |
| TPGYVVTPHTMNLLK | 89 |
| FFTAICDMVAWLGYTPYK | 90 |
| FDDTNPEKEEAKFFTAIYDMVTWLGYTPYK | 91 |
| DRPMEESLLLFEAMR | 92 |
| ILQLVATGAVR | 93 |
| AMAVLESLRVIITNFPAAK | 94 |
| SLDIQVPNFPADETK | 95 |
| AMAVLESLRVIITNFPAAKSLDIQVPNFPADETK | 96 |
| TDFKEEPEPGFKRLAWGQPVGLRHTGYVIELQHVVK | 97 |
| AFIHWVSQPLMCEVR | 98 |
| LGYFSVDPDSHQGKLVFNR | 99 |
| KATGILLYGLASR | 100 |
| SFLVSYIASK | 101 |
| DFERECGVGVIVTPEQIEEAVEAAINR | 102 |
| EAATQAQQTLGSTIDKATGILLYGLASR | 103 |

Hence, certain specific embodiments include isolated QRS polypeptides that comprise, consist essentially of, or consist of any one or more of SEQ ID NOS:36-103 (in Tables H and I above), which modulate hematopoiesis, including variants thereof. In certain embodiments, these isolated QRS polypeptide fragments may further comprise 1, 2, 3, 4, 5, 6, 7, 8, 9, 10, 11, 12, 13, 14, 15, 16, 17, 18, 19, or 20 or more of the C-terminal and/or N-terminal residues that surround them, as characterized by their location within the full-length QRS polypeptide. In certain embodiments, these isolated QRS polypeptide fragments may be truncated to contain 1, 2, 3, 4, 5, 6, 7, 8, 9, 10, 11, 12, 13, 14, 15, 16, 17, 18, 19, or 20 fewer of their C-terminal and/or N-terminal residues. Also included are pharmaceutical compositions comprising such QRS polypeptide fragments, and methods of using said polypeptides or compositions to treat a subject in need thereof.

The present invention also contemplates the use of AARS chimeric or fusion proteins for modulating hematopoiesis. As used herein, an AARS “chimeric protein” or “fusion protein” includes an AARS polypeptide or polypeptide fragment linked to either another AARS-polypeptide (e.g., to create multiple fragments), to a non-AARS polypeptide, or to both. A “non-AARS polypeptide” refers to a “heterologous polypeptide” having an amino acid sequence corresponding to a protein which is different from an AARS protein, and which is derived from the same or a different organism. The AARS polypeptide of the fusion protein can correspond to all or a portion of a biologically active AARS amino acid sequence. In certain embodiments, an AARS fusion protein includes at least one (or two) biologically active portion of an AARS protein. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The fusion partner may be designed and included for essentially any desired purpose provided they do not adversely affect the hematopoietic-modulating activity of the polypeptide. For example, in one embodiment, a fusion partner may comprise a sequence that assists in expressing the protein (an expression enhancer) at higher yields than the native recombinant protein. Other fusion partners may be selected so as to increase the solubility of the protein or to enable the protein to be targeted to desired intracellular compartments.

The fusion protein can include a moiety which has a high affinity for a ligand. For example, the fusion protein can be a GST-AARS fusion protein in which the AARS sequences are fused to the C-terminus of the GST sequences. As another example, an AARS polypeptide may be fused to an eight amino acid tag at the C-terminus, such as an L-E-H-H-H-H-H-H (SEQ ID NO:5) tag. In certain specific embodiments, amino acids 1-364 of a YRS polypeptide are fused to a 365-L-E-H-H-H-H-H-H-372 (SEQ ID NO:5) tag at the C-terminus. Such fusion proteins can facilitate the purification and/or identification of an AARS polypeptide. Alternatively, the fusion protein can be an AARS protein containing a heterologous signal sequence at its N-terminus. In certain host cells, expression and/or secretion of AARS proteins can be increased through use of a heterologous signal sequence.

More generally, fusion to heterologous sequences, such as an Fc fragment, may be utilized to remove unwanted characteristics or to improve the desired characteristics (e.g., pharmacokinetic properties) of an AARS polypeptide. For example, fusion to a heterologous sequence may increase chemical stability, decrease immunogenicity, improve in vivo targeting, and/or increase half-life in circulation of an AARS polypeptide.

Fusion to heterologous sequences may also be used to create bi-functional fusion proteins, such as bi-functional proteins that are not only capable of modulating hematopoiesis (e.g., modulating cells from the myeloid, megakaryocyte, erythrocyte, lymphoid and/or endothelial progenitor (EPC) lineages), through the AARS polypeptide, but are also capable of modifying (i.e., stimulating or inhibiting) other pathways through the heterologous polypeptide. Examples of such pathways include, but are not limited to, various immune system-related pathways, such as innate or adaptive immune activation pathways, or cell-growth regulatory pathways, such as angiogenesis. In certain aspects, the heterologous polypeptide may act synergistically with the AARS polypeptide to modulate hematopoietic-related pathways in a subject. Examples of heterologous polypeptides that may be utilized to create a bi-functional fusion protein include, but are not limited to, thrombopoietin (TPO) or TRP peptide agonists, mpl-ligands, cytokines (e.g., IL-11, SDF-1, CXCL-12), chemokines, chemokine receptor ligands (e.g., CXCR-1, CXCR-2, CXCR-4 ligands), adhesion molecules (e.g., NCAM, ICAM-1, VCAM-1, PECAM-1, L1, CHL1, MAG, Nectins), and various hematopoietic growth factors (e.g., vascular endothelial growth factor (VEGF), fibroblast growth factors (FGF) such as FGF-1, FGF-2, and FGF-4, and other FGFR ligands), in addition to biologically active fragments and/or variants thereof. In certain embodiments, these heterologous polypeptides achieve additive or synergistic effects. Without wishing to be bound by any one theory, certain particular embodiments, such as fusion polypeptides of an AARS polypeptide (e.g., YRS) and TPO or other TPO peptide agonist, may achieve synergistic effects in increasing thrombopoiesis because certain AARS polypeptides are believed to increase thrombopoiesis by a TPO-independent mechanism; hence, the two thrombopoietic-stimulatory mechanisms may cooperate synergistically.

Fusion proteins may generally be prepared using standard techniques. For example, DNA sequences encoding the polypeptide components of a desired fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences may be operably linked to suitable transcriptional or translational regulatory elements. The regulatory elements responsible for expression of DNA are typically located 5′ to the DNA sequence encoding the first polypeptide. Similarly, stop codons required to end translation and transcription termination signals are present 3′ to the DNA sequence encoding the second polypeptide.

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Certain embodiments also encompass dimers of AARS polypeptides. Dimers may include, for example, homodimers between two identical AARS polypeptides, heterodimers between two different AARS polypeptides (e.g., a full-length YRS polypeptide and a truncated YRS polypeptide; a truncated YRS polypeptide and a truncated WRS polypeptide), and/or heterodimers between an AARS polypeptide and a heterologous polypeptide. Certain heterodimers, such as those between an AARS polypeptide and a heterologous polypeptide, may be bi-functional, as described herein. Also included are monomers of AARS polypeptides, including isolated AARS polypeptides monomers that do not substantially dimerize with a second AARS polypeptide, whether due to one or more substitutions, truncations, deletions, additions, chemical modifications, or a combination of these alterations. In certain embodiments, monomeric AARS polypeptides possess biological activities, including hematopoiesis-modulating activities, which are not possessed by dimeric or multimeric AARS polypeptide complexes.

Certain embodiments of the present invention also contemplate the use of modified AARS polypeptides, including modifications that improved the desired characteristics of an AARS polypeptide, as described herein. Modifications of AARS polypeptides of the invention include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like. Exemplary modifications also include pegylation of an AARS-polypeptide (see, e.g., Veronese and Harris, Advanced Drug Delivery Reviews 54: 453-456, 2002, herein incorporated by reference).

In certain aspects, chemoselective ligation technology may be utilized to modify truncated AARS polypeptides of the invention, such as by attaching polymers in a site-specific and controlled manner. Such technology typically relies on the incorporation of chemoselective anchors into the protein backbone by either chemical or recombinant means, and subsequent modification with a polymer carrying a complementary linker. As a result, the assembly process and the covalent structure of the resulting protein polymer conjugate may be controlled, enabling the rational optimization of drug properties, such as efficacy and pharmacokinetic properties (see, e.g., Kochendoerfer, Current Opinion in Chemical Biology 9:555-560, 2005).

The truncated and/or variant AARS polypeptides of the invention may be prepared by any suitable procedure known to those of skill in the art, such as by recombinant techniques. For example, AARS polypeptides may be prepared by a procedure including the steps of: (a) preparing a construct comprising a polynucleotide sequence that encodes a truncated AARS polypeptide and that is operably linked to a regulatory element; (b) introducing the construct into a host cell; (c) culturing the host cell to express the truncated AARS polypeptide; and (d) isolating the truncated and/or variant AARS polypeptide from the host cell. In illustrative examples, the nucleotide sequence encodes at least a biologically active portion of a polypeptide sequence set forth in, or derived from, SEQ ID NOS:1, 2, 3, 6, 8, 10, 12, or 14, or a biologically active variant or fragment thereof. Recombinant AARS polypeptides can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6.

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the desired molecule.

### Polynucleotide Compositions

The present invention also provides isolated polynucleotides that encode the aminoacyl-tRNA synthetase polypeptides of the invention, including truncations and/or variants thereof, as well as compositions comprising such polynucleotides.

As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an aminoacyl-tRNA synthetase or a portion thereof) or may comprise a variant, or a biological functional equivalent of such a sequence. Polynucleotide variants may contain one or more substitutions, additions, deletions and/or insertions, as further described below, preferably such that the hematopoietic-modulating activity of the encoded polypeptide is not substantially diminished relative to the unmodified polypeptide. The effect on the hematopoietic-modulating activity of the encoded polypeptide may generally be assessed as described herein.

In additional embodiments, the present invention provides isolated polynucleotides comprising various lengths of contiguous stretches of sequence identical to or complementary to an aminoacyl-tRNA synthetase, wherein the isolated polynucleotides encode a truncated aminoacyl tRNA synthetase as described herein.

Exemplary nucleotide sequences that encode the AARS polypeptides of the application encompass coding sequences, such as the polynucleotide sequences of SEQ ID NOS:4, 7, 9, 11, 13, 15, 17, 19, and 31, as well as portions of the full-length or substantially full-length nucleotide sequences of the AARS genes or their transcripts or DNA copies of these transcripts.

Portions of an AARS nucleotide sequence may encode polypeptide portions or segments that retain the biological activity of the reference polypeptide, including the polypeptides of SEQ ID NOS:1, 2, 3, 6, 8, 10, 12, 14, 16, 25, 28, 30, and 32-108, or polypeptides having an amino acid sequence that is at least 80%, 85%, 90%, 95%, 97%, or 98% identical to these sequences. A portion of an AARS nucleotide sequence that encodes a biologically active fragment of an AARS polypeptide may encode at least about 20, 21, 22, 23, 24, 25, 30, 40, 50, 60, 70, 80, 90, 100, 120, 150, 300 or 400 contiguous amino acid residues, or almost up to the total number of amino acids present in a full-length AARS polypeptide. It will be readily understood that “intermediate lengths,” in this context and in all other contexts used herein, means any length between the quoted values, such as 101, 102, 103, etc.; 151, 152, 153, etc.; 201, 202, 203, etc.

The polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, with the total length preferably being limited by the ease of preparation and use in the intended recombinant DNA protocol.

The invention also contemplates variants of the AARS nucleotide sequences. Nucleic acid variants can be naturally-occurring, such as allelic variants (same locus), homologs (different locus), and orthologs (different organism) or can be non naturally-occurring. Naturally occurring variants such as these can be identified with the use of well-known molecular biology techniques, as, for example, with polymerase chain reaction (PCR) and hybridization techniques as known in the art. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of a reference AARS polypeptide, such as the sequences set forth in SEQ ID NOS: 1, 2, 3, 6, 8, 10, 12, 14, 16, 25, 28, 30, or 32-108. Variant nucleotide sequences also include synthetically derived nucleotide sequences, such as those generated, for example, by using site-directed mutagenesis but which still encode an AARS polypeptide. Generally, variants of a particular AARS nucleotide sequence will have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence as determined by sequence alignment programs described elsewhere herein using default parameters.

AARS nucleotide sequences can be used to isolate corresponding sequences and alleles from other organisms, particularly other organisms or microorganisms. Methods are readily available in the art for the hybridization of nucleic acid sequences. Coding sequences from other organisms may be isolated according to well known techniques based on their sequence identity with the coding sequences set forth herein. In these techniques all or part of the known coding sequence is used as a probe which selectively hybridizes to other AARS-coding sequences present in a population of cloned genomic DNA fragments or cDNA fragments (i.e., genomic or cDNA libraries) from a chosen organism.

Accordingly, the present invention also contemplates polynucleotides that hybridize to reference AARS nucleotide sequences, or to their complements, under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, an AARS polypeptide is encoded by a polynucleotide that hybridizes to a disclosed nucleotide sequence under very high stringency conditions. One embodiment of very high stringency conditions includes hybridizing in 0.5 M sodium phosphate, 7% SDS at 65° C., followed by one or more washes in 0.2×SSC, 1% SDS at 65° C.

Other stringency conditions are well known in the art and a skilled artisan will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula: Tₘ=81.5+16.6 (log₁₀ M)+0.41 (% G+C)−0.63 (% formamide)−(600/length) wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5×SSC, 5×Denhardt's solution (0.1% ficoll, 0.1% polyvinylpyrollidone and 0.1% bovine serum albumin), 0.1% SDS and 200 mg/mL denatured salmon sperm DNA) containing a labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

Polynucleotides and fusions thereof may be prepared, manipulated and/or expressed using any of a variety of well established techniques known and available in the art. For example, polynucleotide sequences which encode polypeptides of the invention, or fusion proteins or functional equivalents thereof, may be used in recombinant DNA molecules to direct expression of a truncated and/or variant aminoacyl-tRNA synthetase polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, expression and/or activity of the gene product.

In order to express a desired polypeptide, a nucleotide sequence encoding the polypeptide, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer or immediate/early cytomegalovirus (CMV) enhancer/promoter region, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol. 55:121-131 (1995)).

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins.

### Antibodies

According to another aspect, the present invention further provides antibodies that exhibit binding specificity for an AARS polypeptide or its cellular binding partner as disclosed herein, or to a portion, variant or derivative thereof, and methods of using same. The term antibody includes the various variations of the same, such as FABs, human antibodies, modified human antibodies, single chains, nonhuman antibodies, and other derivatives of the immunoglobulin fold that underly immune system ligands for antigens, as described herein and known in the art. Antibodies can be used in any of the therapeutic, diagnostic, drug discovery, or protein expression/purification methods and compositions provided herein.

Certain antibodies of the present invention differ from certain previously made antibodies because they can distinguish between certain AARS protein fragments/variants and their corresponding full-length AARS, typically by binding with greater affinity to the AARS protein fragments/variants than to the corresponding full-length AARS. Generally, such antibodies may bind to unique sequences or structures generated or revealed by splice variations, proteolysis, mutation, or other cellular processing that generates an AARS protein fragment/variant of the invention (e.g., post translational processing, including but not limited to phosphorylation and other modifications that change protein structure). For example, such antibodies may have binding specificity to one or more non-solvent exposed faces that are exposed in the AARS protein fragment/variant but not in the full-length AARS, or sequences that are not found or are otherwise inaccessible in the full-length AARS. Antibodies may also bind to unique three-dimensional structures that result from differences in folding between the AARS protein fragment/variant and the full-length AARS. Such differences in folding may be localized (e.g., to a specific domain or region) or globalized. As one example, folding of AARS protein fragments or variants may generate unique continuous or discontinuous epitopes that are not found in the corresponding or parent AARS. Examples also include antibodies that specifically bind to N- or C-termini generated by splice variations, proteolysis, or other cellular processing; such termini may be unique compared to the full-length AARS or may not be exposed for antibody binding in the full-length versions due to their termini being completely or partially buried in the overall structure of the larger AARS parent molecule.

In some embodiments, antibodies provided herein do not form aggregates, have a desired solubility, and/or have an immunogenicity profile that is suitable for use in humans, as described herein and known in the art. Also included are antibodies that are suitable for production work, such as to purify the AARS protein fragments/variants described herein. Preferably, active antibodies can be concentrated to at least about 10 mg/ml and optional formulated for biotherapeutic uses.

In certain embodiments, antibodies are effective for modulating one or more of the hematopoiesis-modulating activities mediated by an AARS polypeptide of the invention. In certain embodiments, for example, the antibody is one that binds to an AARS polypeptide and/or its binding partner, inhibits their ability to interact with each other, and/or antagonizes the hematopoiesis-modulating of the AARS polypeptide. In certain embodiments, for example, the antibody binds to the cellular binding partner of an AARS polypeptide, and mimics the AARS polypeptide activity, such as by increasing or agonizing the hematopoiesis-modulating activity of the AARS polypeptide. Specific embodiments include antibodies that antagonize the thrombopoietic activity of certain YRS polypeptides, such as the exemplary YRS polypeptides of SEQ ID NOS:2 and 3. Accordingly, antibodies may be used to diagnose, treat, or prevent diseases, disorders or other conditions that are mediated by an AARS polypeptide of the invention, such as by antagonizing or agonizing its non-canonical activity (e.g., hematopoietic modulating activity) partially or fully.

An antibody, or antigen-binding fragment thereof, is said to “specifically bind,” “immunologically bind,” and/or is “immunologically reactive” to a polypeptide of the invention if it reacts at a detectable level (within, for example, an ELISA assay) with the polypeptide, and does not react detectably in a statistically significant manner with unrelated polypeptides under similar conditions. In certain instances, a binding agent does not significantly interact with a full-length version of the AARS polypeptide.

Immunological binding, as used in this context, generally refers to the non-covalent interactions of the type which occur between an immunoglobulin molecule and an antigen for which the immunoglobulin is specific. The strength, or affinity of binding such as immunological binding interactions can be expressed in terms of the dissociation constant (K_{d}) of the interaction, wherein a smaller K_{d} represents a greater affinity. Immunological binding properties of selected polypeptides can be quantified using methods well known in the art. See, e.g., Davies et al. (1990) Annual Rev. Biochem. 59:439-473. In certain illustrative embodiments, an antibody has an affinity for an AARS polypeptide of the invention of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In certain embodiments, the affinity of the antibody for an AARS protein fragment is stronger than its affinity for a corresponding full-length AARS polypeptide, typically by about 1.5×, 2×, 2.5×, 3×, 3.5×, 4×, 4.5×, 5×, 6×, 7×, 8×, 9×, 10×, 15×, 20×, 25×, 30×, 40×, 50×, 60×, 70×, 80×, 90×, 100×, 200×, 300×, 400×, 500×, 600×, 700×, 800×, 900×, 1000× or more (including all integers in between). In certain embodiments, an antibody as an affinity for a corresponding full-length or parental AARS protein of at least about 0.05, 0.1, 0.25, 0.5, 0.75, 1, 2, 3, 4, 5, 6, 7, 8, 9, 10, 11, 12, 13, 14, 15, 16, 17, 18, 19, or 20 μM. In certain embodiments, an antibody binds weakly or substantially undetectably to a full-length AARS protein.

An “antigen-binding site,” or “binding portion” of an antibody, refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VelocImmune® platform by Regernerex® (see, e.g., U.S. Pat. No. 6,596,541). Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2. Other libraries suitable for use will be apparent to persons skilled in the art. The polypeptides of this invention may be used in the purification process in, for example, an affinity chromatography step.

An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988)PNAS USA. 85 (16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

Certain embodiments include single domain antibody (sdAbs or “nanobodies”), which refer to an antibody fragment consisting of a single monomeric variable antibody domain (see, e.g., U.S. Pat. Nos. 5,840,526; 5,874,541; 6,005,079, 6,765,087, 5,800,988; 5,874,541; and 6,015,695). Such sdABs typically have a molecular weight of about 12-15 kDa. In certain aspects, sdABs and other antibody molecules can be derived or isolated from the unique heavy-chain antibodies of immunized camels and llamas, often referred to as camelids. See, e.g., Conrath et al., JBC. 276:7346-7350, 2001.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J. Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients. See, e.g., U.S. Pat. Nos. 5,530,101; 5,585,089; 5,693,762; 6,180,370; and 7,022,500.

The antibodies of the present invention can be used in any of the therapeutic, diagnostic, drug discovery, protein purification, and analytical methods and compositions described herein.

### Antibody Alternatives and Other Binding Agents

According to another aspect, the present invention further provides antibody alternatives or other binding agents, such as soluble receptors, adnectins, peptides, peptide mimetics, small molecules, aptamers, etc., that exhibit binding specificity for an AARS polypeptide or its cellular binding partner as disclosed herein, or to a portion, variant or derivative thereof, and compositions and methods of using same. Binding agents can be used in any of the therapeutic, diagnostic, drug discovery, or protein expression/purification, and analytical methods and compositions described herein. Biologic-based binding agents such as adnectins, soluble receptors, avimers, and trinectins are particularly useful.

In certain embodiments, such binding agents are effective for modulating one or more of the hematopoiesis-modulating activities mediated by an AARS polypeptide of the invention. In some embodiments, for example, the binding agent is one that binds to an AARS polypeptide and/or its binding partner, inhibits their ability to interact with each other, and/or antagonizes the hematopoiesis-modulating activity of the AARS polypeptide. In specific embodiments, the binding agent binds to and/or antagonizes the thrombopoietic activity of certain YRS polypeptides, such as the polypeptides of SEQ ID NOS:2 or 3. In certain embodiments, for example, the binding agent binds to the cellular binding partner of an AARS polypeptide, and mimics the AARS polypeptide activity, such as by increasing or agonizing the hematopoiesis-modulating activity mediated by the AARS polypeptide. Accordingly, such binding agents may be used to diagnose, treat, or prevent diseases, disorders or other conditions that are mediated by an AARS polypeptide of the invention, such as by antagonizing or agonizing its non-canonical activity (e.g., hematopoiesis-modulating activity) partially or fully.

A binding agent is said to “specifically bind” to an AARS polypeptide of the invention, or its cellular binding partner, if it reacts at a detectable level (within, for example, an ELISA assay) with the polypeptide or its cellular binding partner, and does not react detectably in a statistically significant manner with unrelated polypeptides under similar conditions. In certain instances, a binding agent does not significantly interact with a full-length version of the AARS polypeptide. In certain illustrative embodiments, a binding agent has an affinity for a hematopoiesis-modulating AARS polypeptide of the invention or its cellular binding partner of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In certain embodiments, the affinity of the binding agent for an AARS protein fragment is stronger than its affinity for a corresponding full-length AARS polypeptide, typically by about 1.5×, 2×, 2.5×, 3×, 3.5×, 4×, 4.5×, 5×, 6×, 7×, 8×, 9×, 10×, 15×, 20×, 25×, 30×, 40×, 50×, 60×, 70×, 80×, 90×, 100×, 200×, 300×, 400×, 500×, 600×, 700×, 800×, 900×, 1000× or more (including all integers in between). In certain embodiments, a binding agent has an affinity for a corresponding full-length AARS protein of at least about 0.5, 1, 2, 3, 4, 5, 6, 7, 8, 9, 10, 11, 12, 13, 14, 15, 16, 17, 18, 19, or 20 μM.

As noted above, “peptides” are included as binding agents. The term peptide typically refers to a polymer of amino acid residues and to variants and synthetic analogues of the same. In certain embodiments, the term “peptide” refers to relatively short polypeptides, including peptides that consist of about 2, 3, 4, 5, 6, 7, 8, 9, 10, 11, 12, 13, 14, 15, 16, 17, 18, 19, 20, 25, 30, 35, 40, 45, or 50 amino acids, including all integers and ranges (e.g., 5-10, 8-12, 10-15) in between, and interact with an AARS polypeptide, its cellular binding partner, or both. Peptides can be composed of naturally-occurring amino acids and/or non-naturally occurring amino acids, as described herein.

In addition to peptides consisting only of naturally-occurring amino acids, peptidomimetics or peptide analogs are also provided. Peptide analogs are commonly used in the pharmaceutical industry as non-peptide drugs with properties analogous to those of the template peptide. These types of non-peptide compound are termed “peptide mimetics” or “peptidomimetics” (Luthman, et al., A Textbook of Drug Design and Development, 14:386-406, 2nd Ed., Harwood Academic Publishers (1996); Joachim Grante, Angew. Chem. Int. Ed. Engl., 33:1699-1720 (1994); Fauchere, J., Adv. Drug Res., 15:29 (1986); Veber and Freidinger TINS, p. 392 (1985); and Evans, et al., J. Med. Chem. 30:229 (1987)). A peptidomimetic is a molecule that mimics the biological activity of a peptide but is no longer peptidic in chemical nature. Peptidomimetic compounds are known in the art and are described, for example, in U.S. Pat. No. 6,245,886.

The present invention also includes peptoids. Peptoid derivatives of peptides represent another form of modified peptides that retain the important structural determinants for biological activity, yet eliminate the peptide bonds, thereby conferring resistance to proteolysis (Simon, et al., PNAS USA. 89:9367-9371, 1992). Peptoids are oligomers of N-substituted glycines. A number of N-alkyl groups have been described, each corresponding to the side chain of a natural amino acid. The peptidomimetics of the present invention include compounds in which at least one amino acid, a few amino acids or all amino acid residues are replaced by the corresponding N-substituted glycines. Peptoid libraries are described, for example, in U.S. Pat. No. 5,811,387.

A binding agent may also include one or more small molecules. A “small molecule” refers to an organic compound that is of synthetic or biological origin (biomolecule), but is typically not a polymer. Organic compounds refer to a large class of chemical compounds whose molecules contain carbon, typically excluding those that contain only carbonates, simple oxides of carbon, or cyanides. A “biomolecule” refers generally to an organic molecule that is produced by a living organism, including large polymeric molecules (biopolymers) such as peptides, polysaccharides, and nucleic acids as well, and small molecules such as primary secondary metabolites, lipids, phospholipids, glycolipids, sterols, glycerolipids, vitamins, and hormones. A “polymer” refers generally to a large molecule or macromolecule composed of repeating structural units, which are typically connected by covalent chemical bond.

In certain embodiments, a small molecule has a molecular weight of less than 1000-2000 Daltons, typically between about 300 and 700 Daltons, and including about 50, 100, 150, 200, 250, 300, 350, 400, 450, 500, 550, 500, 650, 600, 750, 700, 850, 800, 950, 1000 or 2000 Daltons.

Small molecule libraries of chemical and/or biological mixtures, such as fungal, bacterial, or algal extracts, are known in the art and can be screened with any of the assays of the invention. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

Libraries of compounds may be presented in solution (Houghten et al., 1992) or on beads (Lam et al., 1991), on chips (Fodor et al., 1993), bacteria, spores (Ladner et al., U.S. Pat. No. 5,223,409, 1993), plasmids (Cull et al., 1992) or on phage (Cwirla et al., 1990; Devlin et al., 1990; Felici et al., 1991; Ladner et al., U.S. Pat. No. 5,223,409, 1993; Scott and Smith, 1990). Embodiments of the present invention encompass the use of different libraries for the identification of small molecule modulators of one or more AARS protein fragments, their cellular binding partners, and/or their related hematopoiesis-modulating activities. Libraries useful for the purposes of the invention include, but are not limited to, (1) chemical libraries, (2) natural product libraries, and (3) combinatorial libraries comprised of random peptides, oligonucleotides and/or organic molecules.

Chemical libraries consist of structural analogs of known compounds or compounds that are identified as “hits” or “leads” via natural product screening. Natural product libraries are derived from collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. See, e.g., Cane et al., Science 282:63-68, 1998. Combinatorial libraries may be composed of large numbers of peptides, oligonucleotides or organic compounds as a mixture. They are relatively easy to prepare by traditional automated synthesis methods, PCR, cloning or proprietary synthetic methods.

More specifically, a combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

For a review of combinatorial chemistry and libraries created therefrom, see, e.g., Huc, I. and Nguyen, R. (2001) Comb. Chem. High Throughput Screen 4:53-74; Lepre, C A. (2001) Drug Discov. Today 6:133-140; Peng, S. X. (2000) Biomed. Chromatogr. 14:430-441; Bohm, H. J. and Stahl, M. (2000) Curr. Opin. Chem. Biol. 4:283-286; Barnes, C. and Balasubramanian, S. (2000) Curr. Opin. Chem. Biol. 4:346-350; Lepre, Enjalbal, C, et al., (2000) Mass Septrom Rev. 19:139-161; Hall, D. G., (2000) Nat. Biotechnol. 18:262-262; Lazo, J. S., and Wipf, P. (2000) J. Pharmacol. Exp. Ther. 293:705-709; Houghten, R. A., (2000) Ann. Rev. Pharmacol. Toxicol. 40:273-282; Kobayashi, S. (2000) Curr. Opin. Chem. Biol. (2000) 4:338-345; Kopylov, A. M. and Spiridonova, V. A. (2000) Mol. Biol. (Mosk) 34:1097-1113; Weber, L. (2000) Curr. Opin. Chem. Biol. 4:295-302; Dolle, R. E. (2000) J. Comb. Chem. 2:383-433; Floyd, C D., et al., (1999) Prog. Med. Chem. 36:91-168; Kundu, B., et al., (1999) Prog. Drug Res. 53:89-156; Cabilly, S. (1999) Mol. Biotechnol. 12:143-148; Lowe, G. (1999) Nat. Prod. Rep. 16:641-651; Dolle, R. E. and Nelson, K. H. (1999) J. Comb. Chem. 1:235-282; Czarnick, A. W. and Keene, J. D. (1998) Curr. Biol. 8:R705-R707; Dolle, R. E. (1998) Mol. Divers. 4:233-256; Myers, P. L., (1997) Curr. Opin. Biotechnol. 8:701-707; and Pluckthun, A. and Cortese, R. (1997) Biol. Chem. 378:443.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, Ru, 3D Pharmaceuticals, Exton, Pa., Martek Biosciences, Columbia, Md., etc.).

Aptamers are also included as binding agents (see, e.g., Ellington et al., Nature. 346, 818-22, 1990; and Tuerk et al., Science. 249, 505-10, 1990). Examples of aptamers included nucleic acid aptamers (e.g., DNA aptamers, RNA aptamers) and peptide aptamers. Nucleic acid aptamers refer generally to nucleic acid species that have been engineered through repeated rounds of in vitro selection or equivalent method, such as SELEX (systematic evolution of ligands by exponential enrichment), to bind to various molecular targets such as small molecules, proteins, nucleic acids, and even cells, tissues and organisms. See, e.g., U.S. Pat. Nos. 6,376,190; and 6,387,620 Hence, included are nucleic acid aptamers that bind to the AARS polypeptides described herein and/or their cellular binding partners.

Peptide aptamers typically include a variable peptide loop attached at both ends to a protein scaffold, a double structural constraint that typically increases the binding affinity of the peptide aptamer to levels comparable to that of an antibody's (e.g., in the nanomolar range). In certain embodiments, the variable loop length may be composed of about 10-20 amino acids (including all integers in between), and the scaffold may include any protein that has good solubility and compacity properties. Certain exemplary embodiments may utilize the bacterial protein Thioredoxin-A as a scaffold protein, the variable loop being inserted within the reducing active site (-Cys-Gly-Pro-Cys-loop in the wild protein), with the two cysteines lateral chains being able to form a disulfide bridge. Methods for identifying peptide aptamers are described, for example, in U.S. Application No. 2003/0108532. Hence, included are peptide aptamers that bind to the AARS polypeptides described herein and/or their cellular binding partners. Peptide aptamer selection can be performed using different systems known in the art, including the yeast two-hybrid system.

Also included are adnectins™, avimers™, and anticalins that specifically bind to an AARS protein fragment of the invention. Adnectins™ refer to a class of targeted biologics derived from human fibronectin, an abundant extracellular protein that naturally binds to other proteins. See, e.g., U.S. Application Nos. 2007/0082365; 2008/0139791; and 2008/0220049. Adnectins™ typically consists of a natural fibronectin backbone, as well as the multiple targeting domains of a specific portion of human fibronectin. The targeting domains can be engineered to enable an Adnectin™ to specifically recognize a therapeutic target of interest, such as an AARS protein fragment of the invention.

Avimers™ refer to multimeric binding proteins or peptides engineered using in vitro exon shuffling and phage display. Multiple binding domains are linked, resulting in greater affinity and specificity compared to single epitope immunoglobulin domains. See, e.g., Silverman et al., Nature Biotechnology. 23:1556-1561, 2005; U.S. Pat. No. 7,166,697; and U.S. Application Nos. 2004/0175756, 2005/0048512, 2005/0053973, 2005/0089932 and 2005/0221384.

Also included are designed ankyrin repeat proteins (DARPins), which include a class of non-immunoglobulin proteins that can offer advantages over antibodies for target binding in drug discovery and drug development. Among other uses, DARPins are ideally suited for in vivo imaging or delivery of toxins or other therapeutic payloads because of their favorable molecular properties, including small size and high stability. The low-cost production in bacteria and the rapid generation of many target-specific DARPins make the DARPin approach useful for drug discovery. Additionally, DARPins can be easily generated in multispecific formats, offering the potential to target an effector DARPin to a specific organ or to target multiple receptors with one molecule composed of several DARPins. See, e.g., Stumpp et al., Curr Opin Drug Discov Devel. 10:153-159, 2007; U.S. Application No. 2009/0082274; and PCT/EP2001/10454.

Certain embodiments include “monobodies,” which typically utilize the 10th fibronectin type III domain of human fibronectin (FNfn10) as a scaffold to display multiple surface loops for target binding. FNfn10 is a small (94 residues) protein with a β-sandwich structure similar to the immunoglobulin fold. It is highly stable without disulfide bonds or metal ions, and it can be expressed in the correctly folded form at a high level in bacteria. The FNfn10 scaffold is compatible with virtually any display technologies. See, e.g., Batori et al., Protein Eng. 15:1015-20, 2002; and Wojcik et al., Nat Struct Mol. Biol., 2010; and U.S. Pat. No. 6,673,901.

Anticalins refer to a class of antibody mimetics, which are typically synthesized from human lipocalins, a family of binding proteins with a hypervariable loop region supported by a structurally rigid framework. See, e.g., U.S. Application No. 2006/0058510. Anticalins typically have a size of about 20 kDa. Anticalins can be characterized by a barrel structure formed by eight antiparallel β-strands (a stable β-barrel scaffold) that are pairwise connected by four peptide loops and an attached α-helix. In certain aspects, conformational deviations to achieve specific binding are made in the hypervariable loop region(s). See, e.g., Skerra, FEBS J. 275:2677-83, 2008, herein incorporated by reference.

### Modulation of Hematopoiesis and Methods of Use

Embodiments of the present invention relate to the discovery that aminoacyl-tRNA synthetase (AARS) polypeptides, and fragments and variants thereof, modulate hematopoiesis in a variety of useful ways, both in vitro and in vivo. For instance, in certain embodiments, the AARS polypeptides and related agents of the present invention modulate or reduce erythropoiesis, such as by leading to a reduction in the formation of erythroid progenitor cells. As a further example, in certain embodiments, the AARS polypeptides of the present invention modulate or stimulate megakaryopoiesis and/or thrombopoiesis. More generally, AARS polypeptides are capable of modulating cells from the myeloid, megakaryocyte, erythrocyte, granulocyte, lymphoid, thrombocytes, and/or endothelial progenitor (EPC) lineages, among others described herein.

The AARS polypeptides of the present invention may therefore be used to treat various diseases or conditions that benefit from the modulation of hematopoietic processes. Likewise, related agents such as antibodies and other binding agents that interact with these hematopoiesis-regulating AARS polypeptides may also be used to modulate hematopoietic process, and thereby treat or manage diseases and conditions associated with the same, as described herein and known in the art.

“Hematopoiesis” refers generally to the process of cellular differentiation or formation of particular, specialized blood cells from a stem cell or hematopoietic stem cell (HSC). Examples of hematopoietic processes that may be modulated by the AARS polypeptides of the invention include, without limitation, the formation of myeloid cells (e.g., erythroid cells, mast cells monocytes/macrophages, myeloid dendritic cells, granulocytes such as basophils, neutrophils, and eosinophils, megakaryocytes, platelets) and lymphoid cells (e.g., natural killer cells, lymphoid dendritic cells, B-cells, and T-cells).

The methods of modulating hematopoiesis may be practiced in vivo, in vitro, ex vivo, or in any combination thereof. These methods can be practiced on any biological sample, cell culture, or tissue that contains hematopoietic stem cells, hematopoietic progenitor cells, or other stem or progenitor cells that are capable of differentiating along the hematopoietic lineage (e.g., adipose tissue derived stem cells). For in vitro and ex vivo methods, stem cells and progenitor cells, whether of hematopoietic origin or otherwise, can be isolated and/or identified according to the techniques and characteristics described herein and known in the art.

AARS polypeptides and related agents (e.g., antibodies, binding agents) may modulate hematopoiesis in a variety of ways. For instance, AARS polypeptides and related agents may modulate hematopoiesis by directly interacting with a hematopoietic cell or a cell that has the potential to enter the hematopoietic lineage, such as a stem cell. AARS polypeptides and related agents may also modulate hematopoiesis by indirectly altering the tissue microenvironment surrounding a hematopoietic cell or stem cell. In certain embodiments, these relatively indirect mechanisms may involve modulating the activity of any combination of osteoblast cells, vascular cells, and immune cells. For instance, certain embodiments include methods of increasing the hematopoiesis-stimulatory activity of at least one of an osteoblast cell or a vascular cell, reducing the hematopoiesis-stimulatory activity of at least one of an osteoblast cell or a vascular cell, increasing the hematopoiesis-inhibitory activity of at least one of an osteoblast cell or a vascular cell, and reducing the hematopoiesis-inhibitory activity of at least one of an osteoblast cell or a vascular cell. Without wishing to be bound by any one theory, it is believed that the vascular niche, including vascular cells such as endothelial cells, smooth muscle cells, and fibroblasts may play a role in modulating hematopoiesis, and that the osteoblast niche, including osteoblast cells, may also play a role in hematopoietic cell differentiation.

In certain embodiments, AARS polypeptides and related agents remodel the vasculature and/or regulate the necessary interactions between blood vessels and hematopoietic progenitor cells. In these and related embodiments, AARS polypeptides and related agents may be used to treat or manage bone marrow abnormalities, such as those associated with the stroma, the vasculature, bone, blood cells, or bone-marrow micro-environment. As one non-illustrative example, certain bone marrow conditions such as myelodysplastic syndrome involve the abnormal development of hematopoietic progenitors, and antagonistic AARS polypeptides and related agents may reduce or manage this abnormal development. In certain embodiments, WRS polypeptides reduce the abnormal development of hematopoietic progenitors in bone marrow abnormalities such as myelodysplastic syndrome. Additional illustrative bone marrow abnormalities are discussed below.

Also included are methods of increasing the hematopoiesis-stimulatory activity of immune cells such as neutrophils. In these and related embodiments, AARS polypeptides and related agents increase the neutrophil-mediated effect on the release of hematopoietic stem cells and other progenitor cells from the stem cell niche in the bone marrow. Without wishing to be bound by any one theory, it is believed that AARS polypeptides such as YRS and variants (e.g., Y431 mutants, mini-YRS) may stimulate the release of neutrophil proteases which degrade the molecules responsible for anchoring hematopoietic stem cells in the stem cell niche, and thereby facilitate their mobilization into the periphery. Also included are methods of decreasing the hematopoiesis-stimulatory activity of neutrophils. In these and related embodiments, AARS polypeptides such as WRS and variants may decrease the neutrophil-mediated effect on the release of hematopoietic stem cells and other progenitor cells from the stem cell niche in the bone marrow, and thereby reduce their mobilization into the periphery.

Also included are methods of modulating the trafficking or mobilization of hematopoietic cells, including hematopoietic stem cells, progenitor cells, erythrocytes, granulocytes, lymphocytes, megakaryocytes, and thrombocytes. In certain embodiments, these methods increase the trafficking of one or more selected hematopoietic cells between the bone marrow and periphery, and thereby increase the concentration of the one or more selected hematopoietic cells in the periphery. These methods can be practiced in vivo, in vitro, and ex vivo. For instance, in certain embodiments, AARS polypeptides and related agents may be used to increase the concentration of selected peripheral hematopoietic cells in a bone marrow, stem cell, or blood donor prior to removal of those cells from the donor. In certain embodiments, AARS polypeptides and related agents may be used to increase the number of (stem) cells that can be collected for transplantation before a subject undergoes myeloablative radiation treatment.

Certain specific hematopoietic processes include erythropoiesis, granulopoiesis, lymphopoiesis, megakaryopoiesis, thrombopoiesis, and others. “Erythropoiesis” refers generally to the process by which red blood cells (erythrocytes) are produced from HSCs, and includes the formation of erythroid progenitor cells. “Granulopoiesis” refers to generally to the development of the granulocytic white blood cells, neutrophils, eosinophils, and basophils, and includes the formation of granulocyte progenitor cells, such as myelocytes and promelyocytes. “Lymphopoiesis” refers to process by which lymphocytes, such as T-cells and B-cells are produced from HSCs, and includes the formation lymphocyte progenitor cells, such as lymphoblasts. “Megakaryopoiesis” refers generally to the process by which HSCs in the bone marrow differentiate into mature megakaryocytes, and includes the formation of megakaryocyte progenitor cells. “Thrombopoiesis” refers generally to the formation of blood platelets.

“Erythropoiesis” is a carefully ordered sequence of events. Initially occurring in fetal hepatocytes, the process is taken over by the bone marrow in the child and adult. Although multiple cytokines and growth factors are dedicated to the proliferation of the red blood cell, the primary regulator is erythropoietin (EPO). Red blood cell development is initially regulated by stem cell factor (SCF), which commits hematopoietic stem cells to develop into erythroid progenitors. Subsequently, EPO continues to stimulate the development and terminal differentiation of these progenitors. In the fetus, EPO is produced by monocytes and macrophages found in the liver. After birth, EPO is produced in the kidneys; however, Epo messenger RNA (mRNA) and EPO protein are also found in the brain and in red blood cells (RBCs), suggesting the presence of paracrine and autocrine functions.

Erythropoiesis escalates as increased expression of the EPO gene produces higher levels of circulating EPO. EPO gene expression is known to be affected by multiple factors, including hypoxemia, transition metals (Co²⁺, Ni²⁺Mn²⁺), and iron chelators. However, the major influence is hypoxia, including factors of decreased oxygen tension, red blood cell loss, and increased oxygen affinity of hemoglobin. For instance, EPO production may increase as much as 1000-fold in severe hypoxia.

In certain embodiments, the AARS polypeptides and related agents of the present invention reduce erythropoiesis, and may be used to treat a condition associated with increased red blood cells. In certain embodiments, the AARS polypeptides of the present invention increase erythropoiesis, and may be used to treat a condition associated with reduced red blood cells, such as anemia.

In certain embodiments, the AARS polypeptides and related agents of the present invention may modulate erythropoiesis by reducing formation of erythroid progenitors or by reducing the formation of red blood cells. In certain embodiments, the AARS polypeptides may modulate erythropoiesis by increasing or stimulating the formation (i.e., production) of erythroid progenitors or by increasing the formation of red blood cells.

In certain embodiments, these methods may utilize particular AARS polypeptides or selected dosages or forms (e.g., monomers, dimers, oligomers) of AARS polypeptides that both reduce erythropoiesis and increase megakaryopoiesis, including thrombopoiesis (i.e., the formation of platelets). In certain embodiments, depending on the condition to be treated, these methods may utilize particular AARS polypeptides or selected dosages of AARS polypeptides that reduce erythropoiesis without significantly enhancing megakaryopoiesis.

The methods of modulating erythropoiesis may be practiced in vivo, in vitro, ex vivo, or in any combination thereof. In vitro and ex vivo methods can be practiced on any biological sample or cell culture that contains hematopoietic stem cells, or other stem or progenitor cells that are capable of differentiating along the hematopoietic lineage (e.g., adipose tissue derived stem cells). Examples of biological samples include bone marrow, cord blood, and enriched stem cells, in addition to others described herein and known in the art. In certain instances, it may be advantageous to reduce the formation of erythroid progenitor cells in such biological samples or cell cultures.

In certain erythropoiesis-reducing embodiments, merely by way of non-limiting example, AARS polypeptides and related agents may be administered directly to a subject to reduce red blood count, if desired. In this regard, a normal red blood cell count typically ranges from about 4.7 to about 6.1 million red blood cells per μl in men, and about 4.2 to about 5.4 million red blood cells per μl in women. A high red blood cell count is generally defined as more than about 5.72 million red blood cells per μl of blood for men and about 5.03 million red blood cells per μl of blood for women. In children, the threshold for high red blood cell count varies with age and sex. Red blood count may also be reflected by a person's hematocrit (i.e., packed cell volume (PCV) or erythrocyte volume fraction (EVF)), which is the proportion or percentage of blood volume that is occupied by red blood cells. A normal hematocrit is normally about 46% for men and about 38% for women. A higher hematocrit value indicates a greater number of red blood cells. In severe cases, a high red blood cell count can impair circulation and lead to abnormal clotting, among other problems.

Hence, certain embodiments of the present invention relate to methods of administering an AARS polypeptide or a related agent to a subject in need thereof, wherein the subject has an increased red blood count (e.g., greater than about 5.72 million red blood cells per μl of blood for men and about 5.03 million red blood cells per μl of blood for women, often by a clinically or statistically significant amount), or an increased hematocrit (e.g., greater than about 46% for men or about 38% for women, often by a clinically or statistically significant amount). In certain embodiments, administration of an AARS polypeptide to such a subject reduces their red blood cell count or hematocrit. Also included are methods of reducing red blood cells in a subject, and methods of reducing hematocrit in a subject, including a subject that has a higher than normal red blood cell count or hematocrit, or is at risk for developing such a condition, comprising administering to the subject an AARS polypeptide of the present invention, and thereby reducing red blood cell count or hematocrit in the subject.

There are many general diseases or conditions that increase the red blood cell count or hematocrit of a subject, and which may be improved or treated by the AARS polypeptides and related agents of the present invention. As one general, illustrative example, high red blood cell count may result from increases in red blood cell production, mainly to compensate for low oxygen levels, which may be caused by poor heart or lung function. Also, high red blood cell count may result from increased release of erythropoietin (EPO) from the kidneys (EPO enhances red blood cell production), production of too many red blood cells by the bone marrow, impairment of the oxygen-carrying capacity of red blood cells (leading to over-production), compensation for a limited oxygen supply in higher altitudes, and the loss of blood plasma (i.e., the liquid component of blood), which may create relatively high levels of red blood cells, volume-wise.

Further examples of conditions that are associated with high red blood cell count include, without limitation, living at a high altitude, smoking, congenital heart disease, failure of the right side of the heart (i.e., cor pulmonale), scarring and thickening of the lung tissue (i.e., pulmonary fibrosis), bone marrow disorders (e.g., polycythemia vera), dehydration, such as from severe diarrhea or excessive sweating, kidney disease/cancer, exposure to carbon monoxide, anabolic steroid use, COPD or other lung diseases, such as pulmonary fibrosis, and EPO doping, mainly to enhance athletic performance. Hence, the AARS polypeptides can be used to treat or reduce the risk of developing high red blood cell count or volume as it is associated with these or any other conditions known in the art.

Polycythemia refers to an increase in the red blood cell count, hemoglobin, and total red blood cell volume, typically accompanied by an increase in total blood volume. Polycythemia can be distinguished from relative erythrocytosis secondary to fluid loss or decreased intake, because polycythemia results in increased total blood volume, and relative erythrocytosis does not. Two basic categories of polycythemia are typically recognized: primary polycythemias, which are due to factors intrinsic to red cell precursors and include the diagnoses of primary familial and congenital polycythemia (PFCP) and polycythemia vera (PV), and secondary polycythemias, which are caused by factors extrinsic to red cell precursors.

Primary polycythemia refers to a variety of myeloproliferative syndromes that include, for example, polycythemia vera, essential thrombocythemia, agnogenic myeloid metaplasia, and myelofibrosis.

Polycythemia vera has a significant genetic component. For instance, an activating mutation in the tyrosine kinase JAK2 (JAK2^{V617F}) now appears to cause most primary cases in adults. Several other mutations of JAK2 have also been described (e.g., exon 12, JAK2^{H538-K539delinsI}). These and possibly other JAK2 mutations are thought to cause hypersensitivity to EPO via the EPO receptor. Familial clustering suggests a genetic predisposition. Also, the clonality of polycythemia vera is well established. Studies also suggest hypersensitivity of the myeloid progenitor cells to growth factors, including EPO, IL-3, SCF, GM-CSF, and insulin-like growth factor (IGF)-1, whereas other studies show defects in programmed cell death.

PFCP is caused by a hypersensitivity of erythroid precursors to EPO. Several mutations (approximately 14) have been identified in the Epo receptor (EPOR) gene. Most of the identified EPOR mutations (11) cause truncation of the c-terminal cytoplasmic receptor domain of the receptor. These truncated receptors have heightened sensitivity to circulating Epo due to a lack of negative feedback regulation.

Secondary polycythemia may result from functional hypoxia induced by lung disease, heart disease, increased altitude (hemoglobin increase of 4% for each 1000-m increase in altitude), congenital methemoglobinemia, and other high-oxygen affinity hemoglobinopathies stimulating increased EPO production. Secondary polycythemia may also result from increased EPO production secondary to benign and malignant EPO-secreting lesions. Secondary polycythemia may also be a benign familial polycythemia. Chuvash polycythemia, a congenital polycythemia first recognized in an endemic Russian population, has mutations in the von Hippel-Lindau (VHL) gene, which is associated with a perturbed oxygen dependent regulation of EPO synthesis. Secondary polycythemia of the newborn is fairly common and may result from either chronic or acute fetal hypoxia or delayed cord clamping and stripping of the umbilical cord. Accordingly, AARS polypeptides may be used in treating or reducing the risk of primary polycythemia, such as polycythemia vera, or secondary polycythemia.

Also, certain primary treatment regimes may lead to an undesirably increase in red blood cells. For instance, the drugs gentamicin and methyldopa have been associated with increasing the number of red blood cells in a subject. Hence, the AARS polypeptides may be used in conjunction or combination with one or more of gentamicin, methyldopa, or other drug that leads to increased production of red blood cells, mainly to off-set the undesired effects of producing too many red blood cells. In certain embodiments, by reducing their undesirable side effects, combination therapy with AARS polypeptides may allow the use of higher concentrations of gentamicin, methyldopa, or related drugs.

Accordingly, in certain embodiments, the AARS polypeptides or related agents may be used to reduce erythropoiesis, and also to reduce the formation of erythroid progenitors, red blood cells, or both. In certain embodiments, methods of reducing erythropoiesis or red blood cell formation may be used to treat a subject that has or is at risk for having increased red blood cell count, increased hemoglobin levels, or increased total red blood cell volume, as described herein and known in the art.

In certain erythropoiesis-stimulating embodiments, merely by way of non-limiting example, AARS polypeptides and related agents may be administered directly to a subject to increase or maintain red blood count, if desired, such as to treat a condition associated with reduced blood count or risk of reduced blood count. Typically, conditions associated with reduced blood count are referred to as anemias. Hence, certain embodiments may include the use or administration of AARS polypeptides to treat or reduce the risk of anemia, or to treat a condition associated with anemia. Certain embodiments may include the use of AARS polypeptides or related agents to increase erythropoiesis in vitro or ex vivo, such as to increase the number of erythrocyte progenitor cells or red blood cells in a population of hematopoietic cells, which may then be optionally administered to a subject in need thereof.

Anemia may be associated with any one or more of excessive bleeding, reduced production of red blood cells, or increased destruction of red blood cells. For example, aplastic anemia is typically caused by the inability of the bone marrow to produce blood cells, and pure red cell aplasia is typically caused by the inability of the bone marrow to produce only red blood cells. Aplastic anemia can be inherited, can occur without apparent cause, or can occur when the bone marrow is injured by medications, radiation, chemotherapy, or infection. Also included is thalassemia, a condition that occurs when the red cells fail to mature and grow properly. Thalassemia is an inherited condition that typically affects people of Mediterranean, African, Middle Eastern, and Southeast Asian descent. This condition can range in severity from mild to life-threatening; the most severe form is called Cooley's anemia. As a further example, anemia may be caused by lead exposure, which is toxic to the bone marrow, and reduces red blood cell production. Also included are iatrogenic bone marrow disorders.

Hemolytic anemia is typically caused by excessive breakdown of red blood cells. Causes of hemolytic anemia may include any one or more of inherited conditions, such as sickle cell anemia and thalassemia, stressors such as infections, drugs, snake or spider venom, or certain foods, toxins from advanced liver or kidney disease, inappropriate attack by the immune system (called hemolytic disease of the newborn when it occurs in the fetus of a pregnant woman), vascular grafts, prosthetic heart valves, tumors, severe burns, chemical exposure, severe hypertension, and clotting disorders. In certain cases, an enlarged spleen can trap red blood cells and destroy them before they enter the circulation.

Anemia also associates with excessive bleeding, whether acute or chronic. Red blood cells can be lost through bleeding, which can occur slowly over a long period of time, and can often go undetected. Chronic bleeding associated with anemia may result from any one or more of gastrointestinal conditions such as ulcers, hemorrhoids, gastritis (inflammation of the stomach) and cancer, use of nonsteroidal anti-inflammatory drugs (NSAIDS) such as aspirin or Motrin®, as well as menstruation and childbirth in women, especially if menstrual bleeding is excessive and if there are multiple pregnancies.

Certain types of anemia associate with vitamin deficiencies or iron deficiencies. For instance, vitamin deficiency anemia may occur when vitamin B-12 and folate are deficient. These two vitamins are needed to make red blood cells. Conditions leading to anemia caused by vitamin deficiency include any one or more of megaloblastic anemia, in which vitamin B-12 or folate or both are deficient, pernicious anemia, in which poor vitamin B-12 absorption is caused by conditions such as Crohn's disease, an intestinal parasite infection, surgical removal of part of the stomach or intestine, or infection with HIV, dietary deficiency, in which eating little or no meat may cause a lack vitamin B12, or overcooking or eating too few vegetables may cause a folate deficiency, and other causes, such as pregnancy, certain medications, alcohol abuse, and intestinal diseases such as tropical sprue and gluten-sensitive enteropathy (celiac disease). During early pregnancy, sufficient folic acid can prevent the fetus from developing neural tube defects such as spina bifida.

Anemia also associates with other conditions, and usually occurs when there are too few of the hormones required for red blood cell production. Conditions causing this type of anemia include, for example, advanced kidney disease, hypothyroidism, cancer, infection (e.g., bacterial, viral, parasitic), and autoimmune disorders such as lupus and rheumatoid arthritis.

Certain embodiments may include combination therapies for treating anemias, including the administration of one or more AARS polypeptides in combination with other anemia-based therapeutic agents or treatment modalities. Examples of combination therapies included, without limitation, any one or more of iron supplementation with ferrous sulfate, ferrous gluconate, or vitamin C, the latter of which may aid in the body's ability to absorb iron, vitamin supplements given orally (e.g., folic acid) or subcutaneously (e.g., vitamin B-12), administration of recombinant erythropoietin or epoetin alfa, blood transfusion, or hyperbaric oxygenation.

In certain embodiments, AARS polypeptides and related agents may be used to modulate granulopoiesis. These embodiments may be practiced in vitro, ex vivo, and in vivo. In certain embodiments, the AARS polypeptides and related agents of the present invention may stimulate granulopoiesis, and may be used to treat a condition associated with any one or more of neutropenia, eosinopenia, or basopenia. In certain embodiments, the AARS polypeptides and related agents of the present invention may reduce granulopoiesis, and may be used to treat a condition associated with any one or more of neutrophilia, eosinophilia, or basophilia. In certain in vitro or ex vivo embodiments, AARS polypeptides and related agents may increase or reduce the number of granulocytes (e.g., neutrophils, eosinophils, basophils) in a population of hematopoietic cells, which may then be optionally administered to a subject in need thereof.

Neutropenia can develop if neutrophils are used up or destroyed in the bloodstream faster than the bone marrow can make new neutrophils. Neutrophils are destroyed faster than they are produced in certain bacterial infections, allergic disorders, and drug treatments. Certain autoimmune diseases may lead to the production of antibodies that destroy neutrophils, and thereby associate with neutropenia. Low neutrophil count may also result from an enlarged spleen, because the enlarged spleen traps and destroys neutrophils.

Neutropenia can also develop if the production of neutrophils in the bone marrow is reduced. Examples of conditions associated with reduced neutrophil production include cancer, viral infections such as influenza, bacterial infections such as tuberculosis, myelofibrosis, and deficiencies of vitamin B₁₂ or folate (folic acid). Radiation therapy may also associate with neutropenia, especially if targeted to the bone marrow. Certain drugs, including phenyloin, chloramphenicol, sulfa drugs, chemotherapeutic agents, as well as certain toxins (benzene and insecticides) can also impair the bone marrow's ability to produce neutrophils, and thereby associate with neutropenia. Neutropenia can also result from the colonization of intracellular neutrophilic parasites. Aplastic anemia and various leukemias may also associate with neutropenia. Also included are congenital neutropenia, autosomal recessive Kostmann's syndrome, cyclic neutropenia, and myelokathexis.

Neutrophilia may associate with bacterial infections, any form of acute inflammation, including after a heart attack or other infarct, and the administration of certain drugs, such as prednisone and cortisol, which cause marginated neutrophils to enter the blood stream. Nervousness or emotional stress may also slightly raise the neutrophil count because of this same effect. Neutrophilia also associates with malignancies. For instance, chronic myelogenous leukemia (CML or chronic myeloid leukemia) is characterized by excessive blood cell proliferation, including excessive neutrophil proliferation. Neutrophilia may also associate with eclampsia, gout, thyroiditis, rheumatic fever, appendicitis, vasculitis, trauma, surgery, burns, blood loss, steroids, fungal infection, pregnancy, connective tissue disease, arthritis, dermatitis, hemolytic anemia, and essential thrombocythemia, among other conditions known in the art.

Eosinopenia may associate with steroid use (e.g., Cushing's syndrome), infections (e.g., bacterial infections and sepsis, for which eosinophil count can be a valuable predictor), and psychological stress, among other conditions known in the art. Eosinophilia may be characterized as primary or secondary, or it may be characterized as reactive (i.e., in response to other stimuli such as allergy or infection) or non reactive. Generally, eosinophilia may associate with neoplasia (e.g., lymphoma such as Hodgkin lymphoma and non-Hodgkin lymphoma, human T-cell lymphotropic virus I (HTLV-I) infection, adult T-cell leukemia/lymphoma (ATLL), eosinophilic leukemia, gastric or lung carcinoma), Addison Disease, allergy/asthma, collagen vascular diseases, cholesterol emboli, and parasites. Particular examples of conditions that associate with eosinophilia include, without limitation, coccidioidomycosis fungal infection, hypereosinophilic syndrome, parasitic infections (intestinal helminthiasis), allergic disorders (including eosinophilic esophagitis), certain drug reactions (e.g., DRESS syndrome), cholesterol embolization, Churg-Strauss syndrome, certain forms of chronic myeloid leukemia, Hodgkin's lymphoma, Gleich's syndrome, Addison's disease, Clonorchis sinensis (a flatworm infection), eosinophilia-myalgia syndrome, often caused by contaminated tryptophan supplements, Job's Syndrome, typically caused by increased levels of Immunoglobulin E, and certain forms of colitis, such as eosinophilic colitis.

Basopenia may associate with autoimmune urticaria, a chronic itching condition, and may be an indicator of ovulation. Basophilia may associate with myeloproliferative disorders, such as certain forms of leukemia and lymphoma, including chronic granulocytic leukemia and acute basophilic leukemia, a form of acute myeloid leukemia in which blasts are accompanied by abnormal basophils in all stages of differentiation. Increased basophil counts advanced may be found in advanced anemia, malaria, and chronic lead poisoning. Basophilia may also cause or associate with leukocytosis, or the destruction of white blood cells.

Certain embodiments may include combination therapies for treating any one or more of neutropenia, neutrophilia, eosinopenia, eosinophilia, basopenia, or basophilia, including the administration of one or more AARS polypeptides in combination with other granulocyte-based therapeutic agents or treatment modalities. Examples include the administration of recombinant G-CSF (granulocyte-colony stimulating factor), typically used in treating neutropenia, corticosteroids and interferon (IFN)-alpha, hydroxyurea, chlorambucil, vincristine, cytarabine, 2-chlorodeoxyadenosine (2-CdA), and etoposide, typically used in treating primary eosinophilia.

In certain embodiments, AARS polypeptides and related agents may be used to modulate lymphopoiesis. These embodiments may be practiced in vitro, ex vivo, and in vivo. In certain embodiments, the AARS polypeptides and related agents of the present invention may stimulate lymphopoiesis, and may be used to treat a condition associated with lymphocytopenia. Certain embodiments may be used to treat any one or more of T-lymphocytopenia, B lymphocytopenia, and NK lymphocytopenia. In certain embodiments, the AARS polypeptides of the present invention may reduce lymphopoiesis, and may be used to treat a condition associated with lymphocytosis. In certain in vitro or ex vivo embodiments, AARS polypeptides may increase or reduce the number of lymphocytes (e.g., B-cells, T-cells, NK cells) in a population of hematopoietic cells, which may then be optionally administered to a subject in need thereof.

Various disorders and conditions, including infection with human immunodeficiency virus (HIV), the virus that causes AIDS, associate with decreased numbers of lymphocytes in the blood. Other viral, bacterial, and fungal agents may associate with lymphocytopenia, such as viral hepatitis, tuberculosis, and typhoid fever. Sepsis may also associate with reduced lymphocytes. Lymphocytopenia may associate with starvation, malnutrition, severe stress, intense or prolonged physical exercise, often due to increased cortisol release, autoimmune disorders such as lupus and rheumatoid arthritis, bone marrow or blood malignancies (e.g., leukemia, Hodgkin's disease, aplastic anemia), use of corticosteroids (such as prednisone), use of chemotherapeutics (e.g., cytotoxic agents, immunosuppressive drugs), and radiation therapy or exposure. Severe reduction in lymphocytes can also occur in certain hereditary or congenital disorders, which are often X-linked disorders, such as DiGeorge anomaly, Wiskott-Aldrich syndrome, severe combined immunodeficiency syndrome, and ataxia-telangiectasia.

Lymphocytosis may associate with any one or more of chronic bacterial infections such as pertussis, chronic lymphocytic leukemia, acute lymphoblastic leukemia, multiple myeloma, mumps, ulcerative colitis, vasculitis, Crohn's disease, and whooping cough. Also included are viral infections, such as infectious mononucleosis (glandular fever), Epstein-Barr virus infection, cytomegalovirus (CMV), and hepatitis, protozoal infections, such as toxoplasmosis and American trypanosomiasis (Chagas disease), and chronic intracellular bacterial infections such as tuberculosis and brucellosis. Certain medications, including corticosteroids, lithium and beta agonists, may also cause lymphocytosis.

In certain embodiments, AARS polypeptides and related agents may be used to modulate megakaryopoiesis, thrombopoiesis, or both. These embodiments may be practiced in vitro, ex vivo, and in vivo. In certain embodiments, the AARS polypeptides and related agents of the present invention may stimulate megakaryopoiesis, stimulate thrombopoiesis, or both, and may be used to treat a condition associated with megakaryocytopenia, thrombocytopenia, or both. In certain embodiments, the AARS and related agents polypeptides of the present invention may reduce megakaryopoiesis, reduce thrombopoiesis, or both, and may be used to treat a condition associated with excess megakaryocytes, excess thrombocytes (e.g., thrombocythemia, thrombocytosis), or both. In certain in vitro or ex vivo embodiments, AARS polypeptides and related agents may increase or reduce the number of megakaryocytes, megakaryocyte progenitors, or platelets in a population of hematopoietic cells, which may then be optionally administered to a subject in need thereof. In certain embodiments, AARS polypeptides and related agents increase thrombopoiesis by a thrombopoietin (TPO)-independent mechanism. In certain preferred embodiments, YRS-based polypeptides, such as those that comprise a Y341A mutation or that comprise or consist essentially of the sequence of mini-YRS (SEQ ID NO:3), increase thrombopoiesis by a TPO-independent mechanism. In certain embodiments, antagonists to Y341A or mini-YRS may be used to reduce thrombopoiesis, or to reduce platelet levels, and thereby treat conditions such as thrombocythemia or thrombocytosis. Examples of antagonists include various antibodies and other binding agents that antagonize the thrombopoietic or megakaryopoeitic activities of Y341A and/or mini-YRS, as described elsewhere herein.

In certain embodiments, these methods may be utilized to either enhance or reduce the growth, differentiation, migration, or accumulation of megakaryocyte progenitor cells, including early progenitor cells, i.e., the most primitive lineage-restricted progenitors of the megakaryocyte lineage, late progenitor cells, or both. In certain embodiments, the methods provided herein may impact (i.e., enhance or reduce) the proliferation, cell cycle changes, mobilization, migration, attachment, cell-cell contacts, endomitosis, or polyploidy of megakaryocyte precursors, megakaryoblasts, or megakaryocytes. In certain embodiments, depending on the particular AARS polypeptides or dosages, these methods may selectively enhance the formation of early megakaryocyte progenitor cells. In certain embodiments, these methods may selectively enhance the formation of late megakaryocyte progenitor cells. In certain embodiments, depending on the particular AARS polypeptides or dosages, these methods may selectively reduce the formation of early megakaryocyte progenitor cells. In certain embodiments, these methods may selectively modulate (e.g., reduce) the formation of late megakaryocyte progenitor cells. The methods may be practiced in vivo, in vitro, ex vivo, or in any combination thereof.

The methods provided herein may also enhance or reduce platelet formation or cell division. For instance, certain methods relate to modulating the transition from pro-platelets (i.e., compartmentalization of mature megakaryocytes) to platelets, and their release into the circulation. Certain AARS polypeptides and related agents may increase the transition from pro-platelets to platelets, and thereby increase the release of platelets into the circulation. In certain embodiments, YRS polypeptides increase the transition from pro-platelets to platelets. Certain AARS polypeptides and related agents may reduce the transition from pro-platelets to platelets, and thereby reduce the release of platelets into the circulation. In certain embodiments, WRS polypeptides or YRS polypeptide antagonists reduce the transition from pro-platelets to platelets.

Also, certain methods relate to modulating the cell division of platelets, which are believed to undergo cell division even in the absence of a nucleus. Certain embodiments therefore relate to the use of AARS polypeptides to increase the cell division of platelets. In certain embodiments, a YRS polypeptide increases platelet cell division. These methods can be used, for example, to increase the number of platelets in a platelet transfusion prior to administration to a donor, and/or to treat or manage a condition associated with reduced platelet levels. Certain embodiments relate to the use of AARS polypeptides to reduce the cell division of platelets. In certain embodiments, a WRS polypeptide or a YRS polypeptide antagonist reduces platelet division. The methods may be practiced in vivo, in vitro, ex vivo, or in any combination thereof.

Included are in vitro or ex vivo methods of modulating megakaryopoiesis. In certain embodiments, these methods relate to stimulating the proliferation or accumulation of megakaryocyte progenitor cells, comprising incubating a culture of hematopoietic stem cells or other blood cells with an aminoacyl-tRNA synthetase polypeptide (i.e., contacting a cell with an AARS polypeptide or related agent), typically for a time sufficient to allow proliferation or accumulation of megakaryocyte progenitor cells, thereby stimulating megakaryopoiesis. In certain embodiments, the progenitor cells include early megakaryocyte progenitor cells, and in certain embodiments they include late megakaryocyte progenitor cells. In these and related embodiments, the AARS polypeptides and related agents of the invention may be incubated with purified HSCs, partially purified HSCs, whole bone marrow cultures (e.g., for bone marrow transplants), cord blood, or other types of blood or marrow-based cultures, such as those used in hematopoietic graft therapies. Such methods may result in a culture that is enriched for early megakaryocyte progenitor cells, late progenitor cells, or both, and which may be administered to a subject in need thereof (e.g., transplant subject), if desired.

Growth or proliferation (or lack thereof) of megakaryocyte progenitor cells (e.g., early, intermediate, late, etc.) can be measured according to routine techniques known in the art and described herein (see, e.g., Example 10). For instance, among other characteristics, early megakaryocyte progenitors may be identified by immuno-staining as Lin⁻c-Kit⁺CD41⁺, and later stage megakaryocyte progenitors may be identified as Lin⁻c-Kit⁻CD41⁺ (see, e.g., Perez et al., PLoS ONE. 3:e3565, 2008; and Lefebvre et al., Journal of Hematotherapy & Stem Cell Research. 9:913-921, 2000, each of which is incorporated by reference in its entirety).

Megakaryocyte progenitor cells are positive for CD34 expression. CD34 is a monomeric cell surface antigen with a molecular mass of approximately 110 kD that is selectively expressed on human hematopoietic progenitor cells. The gene is expressed by small vessel endothelial cells in addition to hematopoietic progenitor cells and is a single-chain 105-120 kDa heavily O-glycosylated transmembrane glycoprotein.

Megakaryocyte progenitor cells also typically express the tetraspanin CD9 antigen. The CD9 antigen is a 227-amino acid molecule with 4 hydrophobic domains and 1 N-glycosylation site. The antigen is widely expressed, but is not present on certain progenitor cells in the hematopoietic lineages. CD9 interacts with the integrin family and other membrane proteins, and is postulated to participate in cell migration and adhesion.

Megakaryocyte progenitor cells may also express CD41, also referred to as the glycoprotein IIb/IIIa integrin, which is the platelet receptor for fibrinogen and several other extracellular matrix molecules. GP Ma is a protein of 788 amino acids, including a 26-residue amino terminal signal peptide, a 29-residue transmembrane domain near the carboxy terminus, and 4 tandemly repeated cysteine-rich domains of 33-38 residues.

Megakaryocyte progenitor cells are typically positive for expression of CD117. CD117 is also known as the receptor tyrosine kinase c-Kit. This receptor has been particularly implicated with stem cells, including hematopoietic stem cells. Multiple isoforms of c-Kit also exist as a result of alternate mRNA splicing, proteolytic cleavage and the use of cryptic internal promoters in certain cell types. Structurally, c-Kit contains five immunoglobulin-like domains extracellularly and a catalytic domain divided into two regions by a 77 amino acid insert intracellularly.

Megakaryocyte progenitor cells are typically positive for expression of CD38. CD38 is a 300-amino acid type II transmembrane protein with a short N-terminal cytoplasmic tail and 4 C-terminal extracellular N-glycosylation sites. This marker is also generally associated with lymphocytes, myeloblasts, and erythroblasts.

Megakaryocyte progenitor cells may also have the phenotype of lacking expression of certain lineage specific markers. For staining purposes a cocktail of binding reagents, herein designated “lin” may be used. A “lin” panel may comprise binding reagents (e.g., antibodies and functional binding fragments thereof, ligands, peptidomimetics) that recognize two or more of the lineage markers. A lin panel will generally include at least one marker expressed on mature B cells, on mature T cells, on mature granulocytes and on mature macrophages. Markers suitable for use in a lineage panel are typically expressed on these mature cells, but are not present on multiple lineages, or on stem and progenitor cells. Lineage markers may include CD2; CD3; CD4; CD7; CD8; CD10; CD11b; CD14; CD19; CD20; CD56; and glycophorin A (GPA) in humans and CD2; CD3; CD4; CD8; CD19; IgM; Ter110; Gr-1 in mice. Megakaryocyte progenitor cells are also typically negative for expression of Thy-1 (CD90), which is a 25-35 kD molecule expressed on 1-4% of human fetal liver cells, cord blood cells, and bone marrow cells.

“Hematopoietic stem cells (HSCs)” relate generally to either pluripotent or multipotent “stem cells” that give rise to the blood cell types, including myeloid (e.g., monocytes/macrophages, neutrophils, basophils, eosinophils, erythrocytes, megakaryocytes/platelets, dendritic cells), and lymphoid lineages (e.g., T-cells, B-cells, NK-cells), and others known in the art. “Stem cells” are typically defined by their ability to form multiple cell types (i.e., multipotency) and their ability to self-renew. In certain embodiments, however, oligopotent and unipotent progenitors may be included.

HSCs may be obtained according to known techniques in the art. For instance, HSCs may be found in the bone marrow of adults, which includes femurs, hip, ribs, sternum, and other bones. HSCs may be obtained directly by removal from the hip using a needle and syringe, or from the blood, often following pre-treatment with cytokines, such as G-CSF (granulocyte colony-stimulating factors), that induce cells to be released from the bone marrow compartment. Other sources for clinical and scientific use include umbilical cord blood, placenta, and mobilized peripheral blood. For experimental purposes, fetal liver, fetal spleen, and AGM (Aorta-gonad-mesonephros) of animals are also useful sources of HSCs.

HSCs may be identified according to certain phenotypic or genotypic markers. For example, HSCs may be identified by their small size, lack of lineage (lin) markers, low staining (side population) with vital dyes such as rhodamine 123 (rhodamine^{DULL}, also called rho^{lo}) or Hoechst 33342, and presence of various antigenic markers on their surface, many of which belong to the cluster of differentiation series (e.g., CD34, CD38, CD90, CD133, CD105, CD45, and c-kit, the receptor for stem cell factor). HSCs are mainly negative for the markers that are typically used to detect lineage commitment, and, thus, are often referred to as lin(−) cells. Most human HSCs may be characterized as CD34⁺, CD59⁺, Thy1/CD90⁺, CD38^{lo/-}, C-kit/CD117⁺, and lin(−). However, not all stem cells are covered by these combinations, as certain HSCs are CD34⁻/CD38⁻. Also some studies suggest that earliest stem cells may lack c-kit on the cell surface. For human HSCs, CD133 may represent an early marker, as both CD34′ and CD34⁻HSCs have been shown to be CD133⁺.

For purification of lin(−) HSCs by flow cytometry, or FACS, an array of mature blood-lineage marker antibodies may be used to deplete the lin(+) cells or late multipotent progenitors (MPP), including, for example, antibodies to CD13 and CD33 for human myeloid cells, CD71 for human erythroid cells, CD19 for human B cells, CD61 for human megakaryocytic cells, Mac-1 (CD11b/CD18) for monocytes, Gr-1 for Granulocytes, Il7Ra, CD3, CD4, CD5, and CD8 for T cells, among others known in the art. Other purification methods are known in the art, such as those methods that use the particular signature of the ‘signaling lymphocyte activation molecules’ (SLAM) family of cell surface molecules.

HSCs, whether obtained from, or present in, cord blood, bone marrow, peripheral blood, or other source, may be grown or expanded in any suitable, commercially available or custom defined medium, with or without serum, as desired (see, e.g., Hartshorn et al., Cell Technology for Cell Products, pages 221-224, R. Smith, Editor; Springer Netherlands, 2007, herein incorporated by reference in its entirety). For instance, in certain embodiments, serum free medium may utilize albumin and/or transferrin, which have been shown to be useful for the growth and expansion of CD34+ cells in serum free medium. Also, cytokines may be included, such as Flt-3 ligand, stem cell factor (SCF), and thrombopoietin (TPO), among others. HSCs may also be grown in vessels such as bioreactors (see, e.g., Liu et al., Journal of Biotechnology 124:592-601, 2006, herein incorporated by reference in its entirety). A suitable medium for ex vivo expansion of HSCs may also comprise HSC supporting cells, such as stromal cells (e.g., lymphoreticular stromal cells), which can be derived, for instance, from the disaggregation of lymphoid tissue, and which have been show to support the in vitro, ex vivo, and in vivo maintenance, growth, and differentiation of HSCs, as well as their progeny.

HSC growth or expansion can be measured in vitro or in vivo according to routine techniques known in the art. For example, WO 2008/073748, herein incorporated by references for these methods, describes methods for measuring in vivo and in vitro expansion of HSCs, and for distinguishing between the growth/expansion of HSCs and the growth/expansion of other cells in a potentially heterogeneous population (e.g., bone marrow), such as intermediate progenitor cells. The administering or incubation step that results in the growth or expansion can occur in vivo, ex vivo, or in vitro, though in certain embodiments, the administration or incubation occurs during ex vivo treatment of HSCs.

“Cord blood” or “umbilical cord blood” relates generally to the relatively small amount of blood (up to about 180 mL) from a newborn baby that returns to the neonatal circulation if the umbilical cord is not prematurely clamped. Cord blood is rich in HSCs, and may be harvested and stored for later use according to techniques known in the art (see, e.g., U.S. Pat. Nos. 7,147,626 and 7,131,958, herein incorporated by reference for such methodologies). Also, if the umbilical cord is ultimately not clamped, a physiological clamping occurs upon interaction with cold air, wherein the internal gelatinous substance, called Wharton's jelly, swells around the umbilical artery and veins. Nonetheless, Wharton's jelly can still serve as a source of HSCs.

However, delayed platelet recovery is an inherent problem with cord blood cell transplantation. In this regard, rapid platelet recovery after transplant reduces the cost of supportive therapy and reduces the risk of fatal bleeding due to severe thrombocytopenia. Delayed platelet recovery in cord blood transplantation is associated with low numbers of megakaryocyte progenitor cells in cord blood grafts (see, e.g., Kanamaru et al., Stem Cells. 18:190-195, 2000). Hence, methods of ex vivo pre-treatment of cord blood grafts with AARS polypeptides, methods of in vivo administration of AARS polypeptides prior to, during, or after cord blood transplantation, or both methods in combination, may increase the number of megakaryocyte progenitor cells, increase platelet recovery in cord blood transplantation, and thereby reduce secondary costs and improve the therapeutic outcome of such transplant procedures.

As noted above, “ex vivo” refers generally to activities that take place outside an organism, such as experimentation or measurements done in or on living tissue in an artificial environment outside the organism, preferably with minimum alteration of the natural conditions. Most commonly, “ex vivo” procedures involve living cells or tissues taken from an organism and cultured in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The terms “ex vivo administration,” “ex vivo treatment,” or “ex vivo therapeutic use,” relate generally to medical procedures in which one or more organs, cells, or tissues are obtained from a living or recently deceased subject, optionally purified/enriched, exposed to a treatment or procedure to expand the stem cells (e.g., an ex vivo administration step that involves incubating the cells with a composition of the present invention to enhance expansion of desirable cells, such as HSCs or megakaryocyte progenitors), and then administered to the same or different living subject after that optional treatment or procedure. As one example, thrombocytopenia may be alleviated by infusion of megakaryocyte progenitor cells (see, e.g., De Bruyn et al., Stem Cells Dev. 14:415-24, 2005, herein incorporated by reference).

Such ex vivo therapeutic applications may also include an optional in vivo treatment or procedural step, such as by administering an AARS polypeptide of the invention one or more times to the living subject prior to, during, or after administration of the organ, cells, or tissue. Both local and systemic administration are contemplated for these embodiments, according to well-known techniques in the art. The amount of AARS polypeptide administered to a subject will depend on the characteristics of that subject, such as general health, age, sex, body weight, and tolerance to drugs, as well as the degree, severity, and type of reaction to the polypeptide and/or cell transplant.

Megakaryocytic progenitors can be generated ex vivo, as described herein, and administered to any subject in need thereof, including, for example, subjects having or at risk for developing reduced platelet or thrombocytopenia. Thrombocytopenia is generally characterized by reduced platelet counts, as compared to a normal range of platelet counts for a typical subject. For example, thrombocytopenia refers generally to a decrease in the platelet count to about 150,000/mm³ or lower compared to a normal platelet count. A normal platelet count generally ranges from about 150,000 mm³ to about 450,000 mm³ in a subject.

Thrombocytopenia often causes no signs or symptoms, but may be identified by routine blood tests. If present, possible signs and symptoms of thrombocytopenia include easy bruising and/or excessive bleeding. For example, bleeding in the skin may be the first sign of a low platelet count. Many tiny red dots (petechiae) often appear in the skin on the lower legs, and minor injuries may cause small scattered bruises. In addition, the gums may bleed, and blood may appear in the stool or urine. Menstrual periods may be unusually heavy. Bleeding may be hard to stop.

Bleeding typically worsens as the number of platelets decreases. People who have very few platelets may lose large amounts of blood into the digestive tract or may develop life-threatening bleeding in the brain even though they have not been injured. The rate at which symptoms develop can vary depending on the cause of thrombocytopenia.

Thrombocytopenia may be congenital, acquired, and/or iatrogenic, and may stem from a variety of underlying physiological causes or conditions. For example, thrombocytopenia may result generally from decreased production of platelets, increased destruction of platelets, consumption of platelets, entrapment/sequestration of platelets due to hypersplenism (i.e., enlarged spleen) or hypothermia, and/or from the side-effects of certain medications (i.e., medication induced thrombocytopenia). In addition, idiopathic forms of thrombocytopenia occur, especially in children, transient forms may follow viral infections (e.g., Epstein-Barr or infectious mononucleosis), and pregnant women may develop mild thrombocytopenia, often when close to delivery.

Examples of congenital conditions associated with the decreased production (i.e., diminished or defective production) of platelets include Wiskott-Aldrich syndrome, maternal ingestion of thiazides, congenital amegakaryocytic thrombocytopenia, thrombocytopenia absent radius syndrome, Fanconi anemia, Bernard-Soulier syndrome, May-Hegglin anomaly, Grey platelet syndrome, Alport syndrome, and neonatal rubella. Examples of acquired conditions associated with the decreased production of platelets include aplastic anemia, myelodysplastic syndrome, marrow infiltration (e.g., acute and chronic leukemias, tumors, cancer of the bone marrow), lymphomas, nutritional deficiencies (e.g., B₁₂, folic acid), the use of myelosuppressive agents, the use of drugs that directly influence platelet production (e.g., thiazides, alcohol, hormones), radiation exposure (e.g., radiation therapy), exposure to toxic chemicals (e.g., pesticides, arsenic, benzene), decreased production of thrombopoietin by the liver in liver failure, bacterial sepsis, and certain viral infections (e.g., chickenpox, mumps, parvovirus, measles, dengue, HIV, HCV). AARS polypeptides and ex vivo expanded megakaryocyte progenitors generated therefrom, as described herein, may be used to treat or manage any of these conditions.

Examples of congenital conditions associated with increased peripheral platelet destruction include nonimmune conditions, such as prematurity, erythroblastosis fetalis, infection; and immune conditions, such as drug sensitivity, idiopathic thrombocytopenic purpura (ITP), and maternal ITP. Examples of acquired conditions associated with increased peripheral platelet destruction include nonimmune conditions, such as hemolytic-uremic syndrome, disseminated intravascular coagulation, thrombotic thrombocytopenic purpura (TTP); immune conditions, such as drug-induced thrombocytopenia (e.g., especially with quinine and quinidine), post-transfusion purpura, systemic lupus erythematosus, rheumatoid arthritis, neonatal alloimmune thrombocytopenia, paroxysmal nocturnal hemoglobinuria, acute and chronic ITP, sepsis, and alcohol; in addition to the use of invasive lines and devices (e.g., arterial or central venous catheters), intra-aortic ballon pumps, prosthetic heart valves, as well as the use of heparin-related therapies. AARS polypeptides and ex vivo expanded megakaryocyte progenitors generated therefrom, as described herein, may be used to treat or manage any of these conditions.

Medication-induced thrombocytopenia may result in particular from certain drugs, such as chemotherapeutic agents, nonsteroidal anti-inflammatory agents, sulfonamides, vancomycin, clopidogrel, glycoprotein IIb/IIIa inhibitors, interferons, valproic acid, abciximab, linezolid, famotidine, mebeverine, histamine blockers, alkylating agents, heparin, alcohol, antibiotic chemotherapeutic agents, carbapenems, ureido-penicillins, cefazolin, among others known in the art. Particular examples of chemotherapeutic agents include, but are not limited to, cisplatin (CDDP), carboplatine, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, temazolomide (an aqueous form of DTIC), or any analog or derivative variant of the foregoing. AARS polypeptides and ex vivo expanded megakaryocyte progenitors generated therefrom, as described herein, may be used to treat or manage any of these medication-induced conditions.

The present invention relates generally to methods of treating, or reducing the risks of developing, thrombocytopenia (i.e., decreased platelet count) in a subject, such as in a subject having one or more of the exemplary diseases or conditions provided herein, among others known in the art, by administering to the subject a composition comprising a therapeutically effective concentration of an AARS polypeptide, or by expanding megakaryocyte progenitor cells ex vivo in the presence of AARS polypeptides, and administering those cells to the subject. Embodiments of the present invention encompass methods of treatment intended not only to increase or improving the platelet count in a subject having a reduced, decreased, abnormal, or low platelet count, but to maintain a normal platelet count in a subject at risk for developing a low platelet count. Certain embodiments also contemplate the use of AARS polypeptides to increase the platelet count in a platelet donor, including an otherwise healthy donor (i.e., a donor with a normal platelet count), such as administering an AARS polypeptide to the donor prior to, during, and/or after the platelet donation or apheresis process, or by administering ex vivo expanded megakaryocyte progenitor cells, or both.

Accordingly, certain embodiments include methods for increasing the platelet count in a subject, comprising administering to the subject a composition comprising an AARS polypeptide, or by administering ex vivo or in vitro expanded megakaryocyte progenitor cells (e.g., early or late progenitor cells), thereby increasing the platelet count in the subject. Other embodiments include methods of maintaining a normal platelet count in subject, comprising administering to the subject a composition comprising an AARS polypeptide, or by administering ex vivo or in vitro expanded megakaryocyte progenitor cells, such as wherein the subject is at risk for developing a low platelet count. Certain embodiments may include methods of stimulating thrombopoiesis in a subject, such as by administering to the subject a composition comprising an AARS polypeptide, or by administering ex vivo or in vitro expanded megakaryocyte progenitor cells, or both. In certain aspects, the subject has a reduced, lowered, or abnormal platelet count, such as a platelet count of about 100,000/mm³ or less. In certain aspects, the ARS polypeptides provided herein may be utilized to stimulate the proliferation and/or differentiation of megakaryocytes and/or neutrophils in the subject.

A subject having a reduced platelet count may also be at risk for developing other problems associated with thrombocytopenia, such as bleeding or bruising, hemorrhage, gastrointestinal bleeding, eptistaxis (i.e., nose bleeds), or intracranial hemorrhage (i.e., bleeding in the brain). As one particular example, septic patients with thrombocytopenia have increased bleeding. Accordingly, certain aspects of the invention may utilize the thrombopoietic compositions provided herein to reduce the risk of developing these types of thrombocytopenia associated problems, among others. In other aspects, the subject may be at risk for developing a reduced, lowered, or otherwise abnormal platelet count, such as from an acquired condition associated with lowered platelet levels (e.g., certain medical therapies, leukemias, among others). Also included are surgical patients. For instance, AARS polypeptides may be administered prophylactically, for instance before surgery, to reduce blood loss.

In certain aspects, the methods of treatments described herein may be employed independently of other therapeutic modalities, and may be the only or primary therapeutic modality relied upon to manage a thrombocytopenic condition and/or otherwise reduce the risk not only of developing thrombocytopenia, but of developing other medical problems associated therewith, such as bleeding. For example, a subject having thrombocytopenia for which there is no known, underlying cause (e.g., idiopathic thrombocytopenic purpura), may benefit from the methods of treatment provided herein to increase and/or manage platelet levels.

In certain aspects, the methods and compositions of the present invention may be employed as part of a combination therapy, such as by administration with other agents that may impact thrombopoietic and/or hematopoietic pathways in a subject. Examples of other agents that may be used as part of a combination therapy include thrombopoietin (TPO) and TPO agonists/mimetics, mpl-signaling agonists, cytokines (e.g., IL-11, SDF-1, CXCL-12), chemokines, chemokine receptor ligands (e.g., CXCR-1, CXCR-2, CXCR-4 ligands), adhesion molecules (e.g., NCAM, ICAM-1, VCAM-1, PECAM-1, L1, CHL1, MAG, Nectins), and/or growth factors (e.g., vascular endothelial growth factor (VEGF), fibroblast growth factors (FGF) such as FGF-1, FGF-2, FGF-4, and other FGFR ligands) or other signaling molecules involved in thrombopoiesis or hematopoiesis, including biologically active fragments or variants thereof. In certain embodiments, these combination therapies achieve additive or synergistic effects. Without wishing to be bound by any one theory, certain particular embodiments, such as the combination of an AARS polypeptide (e.g., YRS) and TPO or other TPO peptide agonist or mimetic, achieve synergistic effects in increasing thrombopoiesis because certain AARS polypeptides are believed to increase thrombopoiesis by a TPO-independent mechanism; hence, the two independent thrombopoietic-stimulatory mechanisms may cooperate synergistically to increase thrombopoiesis.

In certain aspects, the methods of the present invention may be employed in conjunction with other therapeutic modalities, such as those involved in treating the underlying condition that causes the condition associated with thrombocytopenia. For example, a subject having congenital amegakaryocytic thrombocytopenia (CAMT) may ultimately undergo a bone marrow transplantation procedure, but may also benefit from a separate treatment, as provided herein, to either enhance platelet levels and/or to maintain platelet levels within a normal range. The thrombopoietic polypeptides of the present invention may be employed in this and similar regards.

In certain aspects, the methods provided herein may be employed in combination with a subject undergoing other medical treatments, such as treatments that either cause thrombocytopenia or increase the risk of developing thrombocytopenia. For example, the methods provided herein may be employed with a subject undergoing, a subject about to undergo, and/or a subject who has undergone, radiation therapy, chemotherapy, or other type of treatment, including various types of pharmaceutical treatments, as described herein and known in the art, since such treatments are known to reduce the platelet count in a subject. Accordingly, the methods provided herein may be utilized before, during, and/or after other medical treatments to reduce the risk of developing thrombocytopenia resulting from such treatments, and/or to manage or improve thrombocytopenia resulting from such treatments. For instance, in certain embodiments, megakaryocytic progenitors can be generated ex vivo and administered to autologous peripheral blood progenitor cell transplant subjects, bone marrow transplant subjects, stem cell transplant subjects, or any other transplant subjects. Examples of such subjects include cancer patients (e.g., breast cancer, non-Hodgkin's lymphoma) undergoing autologous peripheral blood progenitor cell transplant. In these and other embodiments, administration of enriched megakaryocyte progenitors may abrogate the need for allogeneic platelet transfusion support in autologous transplantation (see, e.g., Bertolini et al, Blood. 89:2679-2688, 1997).

As noted above, transfusion of ex vivo expanded megakaryocyte progenitor cells may also be used to shorten the time of platelet recovery in the thrombocytopenia induced by radiotherapy or chemotherapy. In this regard, it has been shown that transfusion of CD34+ cells expanded with TPO+IL-11+heparin (to increase the number of megakaryocyte progenitor cells) into irradiated nonobese diabetic/severe combined immunodeficient mice significantly accelerated platelet recovery (see, e.g., Feng, et al., Experimental Hematology. 33:1537-1543, 2005). In certain embodiments, hematopoietic stem cells (or other biological samples having cells that are capable of differentiating along the hematopoietic lineage) may be expanded ex vivo in the presence of AARS polypeptides, to increase the formation of megakaryocyte progenitors, and then administered to a subject prior to, during, or after radiotherapy or chemotherapy, to increase platelet recovery in the subject. In certain embodiments, AARS polypeptides may be administered directly to such subjects, either separately or in combination with ex vivo treatments.

Accordingly, whether ex vivo or in vitro, AARS polypeptides and related agents may be used in the treatment of cancer. For instance, as noted above, AARS polypeptide-based treatments may be used in combination with chemotherapy, radiotherapy, autologous peripheral blood progenitor cell transplant, bone marrow transplants, or other cancer therapies that impact platelet formation. “Cancer” relates generally to a class of diseases or conditions in which a group of cells display one or more of uncontrolled growth (i.e., division beyond normal limits), invasion (i.e., intrusion on and destruction of adjacent tissues), and metastasis (i.e., spread to other locations in the body via lymph or blood). These malignant properties of cancers differentiate them from benign cancers, which are self-limited, and typically do not invade or metastasize. Also included are myelodysplastic syndromes.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, do not necessarily form tumors. For those cancers that form tumors, the terms cancer (cell) and tumor (cell) are used interchangeably. Examples of cancers include, without limitation, prostate cancer, breast cancer, colon cancer, rectal cancer, lung cancer, ovarian cancer, testicular cancer, stomach cancer, bladder cancer, pancreatic cancer, liver cancer, kidney cancer, brain cancer, melanoma, non-melanoma skin cancer, bone cancer, lymphoma, leukemia, thyroid cancer, endometrial cancer, multiple myeloma, acute myeloid leukemia, neuroblastoma, glioblastoma, and non-Hodgkin's lymphoma. Also included are “cancer stem cells,” a small population of tumor cells that behave like stem cells (i.e., potential for indefinite self renewal), which are often refractory to therapeutic agents due to their dormancy, and which may contribute to the recurrence of cancer. Specific examples of cancer stem cells include “blast cells,” the circulating precursor cells leading to leukemia (AML). In certain embodiments, AARS polypeptides modulate the growth or differentiation of these and other circulating cells, including circulating immune or hematopoietic cells such as hematopoeitic stem cells.

As noted above, AARS polypeptides or AARS polypeptide-expanded megakaryocyte progenitor cells may be administered in combination with chemotherapeutic agents, for instance, to increase platelet recovery. In certain embodiments, the chemotherapy is high-dose chemotherapy, which is often used in conjunction with CD34+ stem cell transplants (or other hematopoietic progenitor cell transplants). Merely by way of illustration, ex vivo expansion of megakaryocyte progenitor cells may provide a complementary transplant product able to enhance platelet production in patients with neuroblastoma (or other cancer) who undergo transplantation with CD34(+) cells following high-dose chemotherapy. Otherwise, these patients show prolonged delays in platelet recovery. Administration protocols for increasing platelet recovering in chemotherapy can be optimized according to techniques in the art.

Examples of general classes of chemotherapeutic or cytotoxic agents included, without limitation, alkylating agents, anti-metabolites, anthracyclines, anti-tumor antiobiotics, platinums, type I topoisomerase inhibitors, type II topoisomerase inhibitors, vinca alkaloids, and taxanes. Examples of particular chemotherapeutic or cytotoxic agents include, without limitation, chlorambucil, cyclophosphamide, lomustine (CCNU), melphalan, procarbazine, thiotepa, carmustine (BCNU), busulfan, daunorubicin, doxorubicin, idarubicin, epirubicin, mitoxantrone, bleomycin, cisplatin, carboplatin, oxaliplatin, camptothecins, irinotecan, topotecan, amsacrine, etoposide, etoposide phosphate, teniposide, vincristine, vinblastine, vinorelbine, vindesine, paclitaxel, and others described herein and known in the art.

AARS polypeptides and ex vivo expanded megakaryocyte progenitor cells may also be used in other tissue transplant therapies associated with reduced platelet levels. For instance, reduced platelets are common after liver transplantation due to platelet sequestration secondary to hypersplenism, and increasing platelet levels may improve post-transplant recovery. Liver transplants may be used to treat chronic active hepatitis and cirrhosis (from alcoholism, unknown cause, or biliary), biliary atresia, which is an incomplete development of the bile duct, and end-stage liver disease, among other liver-related diseases. Since liver transplantation is often a successful treatment for patients with liver related diseases, AARS polypeptides or ex vivo expanded megakaryocyte progenitor cells produced therefrom (or both) can be used in combination with liver transplants for treating these and other liver diseases. The treatment of other types of liver damage is also contemplated, whether by transplant or by direct treatment with AARS polypeptides, including liver damage related to hepatitis virus infection (e.g., HCV).

As noted above, certain embodiments relate to the use of AARS polypeptides and related agents to reduce the number of megakaryocytes, megakaryocyte progenitors, or platelets, whether in a subject in vivo or in tissue culture in vitro or ex vivo. These and related embodiments may be used to treat conditions associated with increased numbers of any one or more of megakaryocytes, megakaryocyte progenitors, or platelets, such as by reducing thrombopoiesis. Particular embodiments include the use of certain WRS polypeptides to reduce thrombopoiesis or megakaryopoiesis. Specific embodiments include the use of variants of YRS (Y341A) to reduce thrombopoiesis or megakaryopoiesis, particularly those that have been converted from having a thrombopoietic-stimulatory activity to having a thrombopoiesis-reducing activity.

Included are conditions associated with thrombocythemia or thrombocytosis, myeloproliferative conditions in which excess platelets are produced, often due to an increased number of megakaryocytes, leading to abnormal blood clotting or bleeding. In essential thrombocythemia, the platelet count is usually 2 to 4 or more times higher than normal. Thrombocythemia is typically characterized as either primary, for which the cause is not known, or secondary, for which the cause is known. Occasionally, primary thrombocythemia changes into a more serious disorder, such as polycythemia vera or certain types of leukemia. Secondary thrombocythemia may associate with bleeding, removal of the spleen, infections, rheumatoid arthritis, certain cancers, premature destruction of red blood cells (hemolysis), iron deficiency, and sarcoidosis, among other conditions known in the art.

As noted above, also included are direct in vivo methods of modulating hematopoiesis. These direct in vivo methods may be used alone or in combination with other treatments, including in combination with the ex vivo treatments described above. For in vivo treatment of human and non-human subjects, the subject is usually administered a pharmaceutical formulation comprising an AARS polypeptide of the present invention. When used for in vivo therapy, the polypeptides of the subject invention are administered to the patient in therapeutically effective amounts (e.g., amounts that modulate hematopoiesis). The polypeptides may be administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. The polypeptides may be administered parenterally, when possible, at the target cell site, or intravenously. Intravenous or subcutaneous administration of the polypeptide is preferred in certain embodiments.

For parenteral administration, the polypeptides may be formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The polypeptides will typically be formulated in such vehicles at concentrations of about 0.01 mg/ml to about 1 mg/ml to about 10 mg/ml, or more.

Generally, a therapeutically effective amount of polypeptide is administered to a subject or patient. In particular embodiments, the amount of polypeptide administered will typically be in the range of about 0.1 μg/kg to about 0.1 mg/kg to about 50 mg/kg of patient body weight. Depending on the type and severity of the disease, about 0.1 μg/kg to about 0.1 mg/kg to about 50 mg/kg body weight (e.g., about 0.1-15 mg/kg/dose) of polypeptide can be an initial candidate dosage for administration to the patient, whether, for example, by one or more separate administrations, or by continuous infusion. For example, a dosing regimen may comprise administering an initial loading dose of about 4 mg/kg, followed by a weekly maintenance dose of about 2 mg/kg of the polypeptide, or about half of the loading dose. However, other dosage regimens may be useful. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The progress of these and other therapies (e.g., ex vivo therapies) can be readily monitored by conventional methods and assays and based on criteria known to the physician or other persons of skill in the art.

### Formulations and Pharmaceutical Compositions

The compositions of the invention comprise aminoacyl-tRNA synthetase polypeptides, including truncations and/or variants thereof, formulated in pharmaceutically-acceptable or physiologically-acceptable solutions for administration to a cell or an animal, either alone, or in combination with one or more other modalities of therapy. Also included are pharmaceutical compositions that comprise antibodies or alternative binding agents, typically those that agonize or antagonize a hematopoiesis-modulating activity of an AARS polypeptide. It will also be understood that, if desired, the compositions of the invention may be administered in combination with other agents as well, such as, e.g., other proteins or polypeptides or various pharmaceutically-active agents. There is virtually no limit to other components that may also be included in the compositions, provided that the additional agents do not adversely affect the hematopoietic-modulating or other effects desired to be achieved.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

In certain applications, the pharmaceutical compositions disclosed herein may be delivered via oral administration to a subject. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the pharmaceutical compositions disclosed herein parenterally, intravenously, intramuscularly, or even intraperitoneally as described, for example, in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the pharmaceutical compositions may be delivered by intranasal sprays, inhalation, and/or other aerosol delivery vehicles. Methods for delivering genes, polynucleotides, and peptide compositions directly to the lungs via nasal aerosol sprays have been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Stimulation of Thrombopoiesis and Megakaryopoiesis In Vivo

The effects of a tyrosyl-tRNA synthetase polypeptide on thrombopoiesis were measured in vivo. The tyrosyl-tRNA synthetase polypeptide utilized in the experiments described below is a C-terminal truncation that comprises amino acids 1-364 of the full-length human tyrosyl-tRNA. This C-terminally truncated polypeptide was fused to an eight amino acid C-terminal tag (365-L-E-H-H-H-H-H-H-372) (SEQ ID NO:5). The amino acid sequence of the full-length human tyrosyl-tRNA synthetase is set forth in SEQ ID NO:1.

To measure the effects of tyrosyl-tRNA synthetase polypeptides on thrombopoiesis, in a first set of experiments, mice were injected subcutaneously twice daily for seven days with 3 μg/kg of the C-terminally truncated tyrosyl-tRNA synthetase polypeptide. In a second set of experiments, mice were injected twice daily for seven days with 1, 3, and 10 μg/kg of the C-terminally truncated tyrosyl-tRNA synthetase polypeptide.

In a third set of experiments, mice were injected subcutaneously twice daily for six days with (i) 3 and 300 μg/kg of the C-terminally truncated tyrosyl-tRNA synthetase polypeptide, and one single daily injection of (ii) 90 μg/kg thrombopoietin (TPO), and (iii) 250 μg/kg G-CSF.

For the first and second set of experiments described above, the platelet count for each animal was determined upon completion of the administration protocol. For the third set of experiments, bone marrow and spleen histology were examined at the end of the administration protocol.

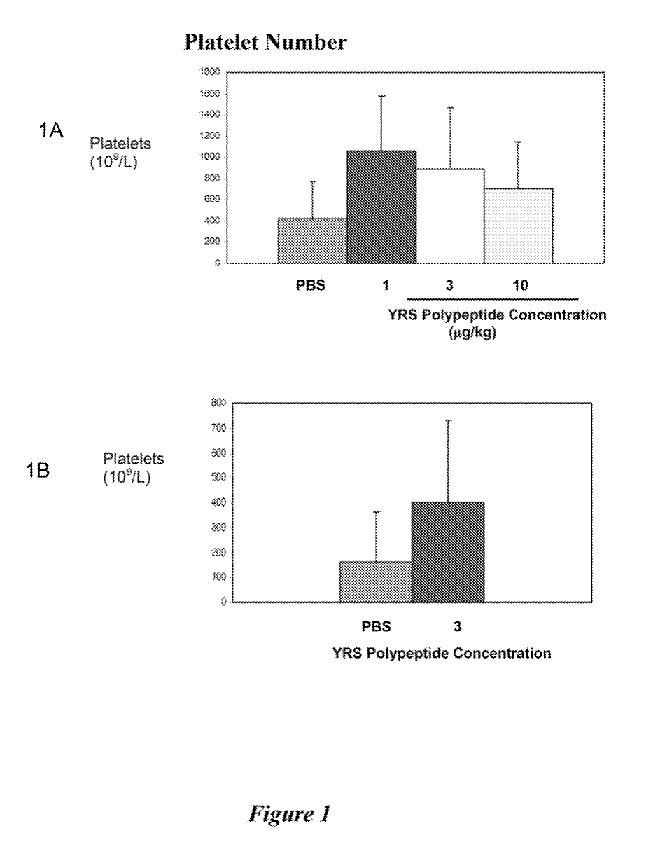

Administration of a truncated tyrosyl-tRNA synthetase for about one week showed a reproducible, in vivo increase in thrombopoietic activity, as measured by either increased platelet count or increased megakaryocyte numbers. FIG. 1(a) shows the platelet count for the experiment in which mice were injected with 1, 3, and 10 μg/kg of the truncated tyrosyl-tRNA synthetase polypeptide, as compared to a phosphate-buffer saline (PBS) control. FIG. 1(b) shows the platelet count for the experiment in which mice were injected with 3 μg/kg of the truncated tyrosyl-tRNA synthetase polypeptide, as compared to a PBS control. In both experiments, mice showed an increase in platelet counts over control in response to treatment with a tyrosyl-tRNA polypeptide of the invention.

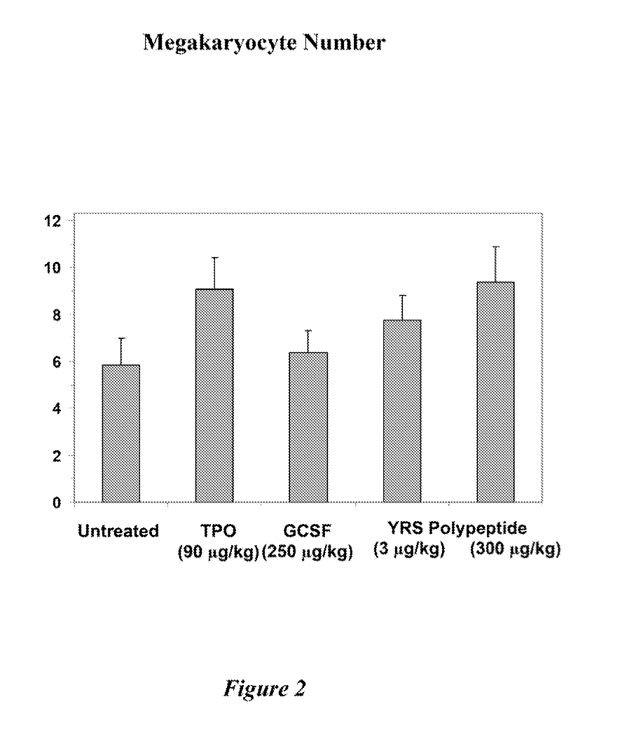

In addition, FIG. 2 shows an increase in megakaryocyte numbers in response to administration of the truncated tyrosyl-tRNA synthetase polypeptide, as compared to untreated animals, which is comparable to the increased numbers observed after administration with TPO. These results show that tyrosyl-tRNA synthetase polypeptide fragments, and in particular C-terminally truncated fragments, are capable of stimulating thrombopoiesis and megakaryopoiesis in vivo.

### Example 2

### In Vitro Measurements of Thrombopoiesis and Megakaryopoiesis

Effects on thrombopoiesis may also be measured in vitro. Stem cells are treated in vitro with a tyrosyl-tRNA synthetase polypeptide of the invention to determine its effect on hematopoietic progenitors of the erythroid, myeloid and megakaryocyte lineages using colony-forming cell (CFC) assays (e.g., inhibition, stimulation, toxicity, synergism with other cytokines, hematopoietic defects). In addition, CD34+ megakaryocyte progenitor cells are treated in vitro with a tyrosyl-tRNA synthetase polypeptide of the invention to monitor megakaryocyte expansion and differentiation (e.g., increase in number of progenitor cells, stimulation of differentiation, increase in polyploidy). Similar experiments are performed using bone marrow and spleen cells derived from mice treated with a tyrosyl-tRNA synthetase polypeptide.

### Example 3

### Combination Therapy Stimulates Thrombopoiesis

To assess whether a tyrosyl-tRNA synthetase polypeptide of the present invention has a synergistic and/or additive effect on the proliferation and differentiation of megakaryocytes in vitro, CD34+ cord blood cells are grown in liquid culture medium in the presence of optimal or sub-optimal formulations of cytokines (StemCell Technologies, Vancouver), such as IL-11, and treated with increasing concentrations of a tyrosyl-tRNA synthetase polypeptide. Additivity or synergism can be determined by monitoring the growth and differentiation of the progenitor cells in the two formulation conditions.

Similarly, in a protocol comparable to that described in Example 1, mice are injected with a limiting amount of thrombopoietin and with increasing amounts of a tyrosyl-tRNA synthetase polypeptide and the effects of the combination therapy on thrombopoiesis in vivo can be determined by platelet and megakaryocyte counts. In addition, combination therapy with limited amounts of other cytokines, chemokines and/or growth factors involved in hematopoiesis can be evaluated using the same type of regimen.

### Example 4

### Thrombopoietic Activity of Tyrosyl-tRNA Synthetase Polypeptides in Rats

The effects of two tyrosyl-tRNA synthetase polypeptides on thrombopoiesis were measured in rats. The tyrosyl-tRNA synthetase polypeptides utilized in the experiments described below are: i) a C-terminal truncation that comprises amino acids 1-364 of the full-length human tyrosyl-tRNA (SEQ ID NO:3) fused to an eight amino acid C-terminal histidine tag (SEQ ID NO:5) and; ii) a mutant of the full length human tyrosyl-tRNA synthetase with a single, tyrosine to alanine, amino acid substitution at position 341, referred to as “Y341A” (SEQ ID NO: 2).

To measure the effects of tyrosyl-tRNA synthetase polypeptides on thrombopoiesis, platelet count for each rat was determined one day before the first scheduled injection and animals were grouped in seven cohorts according to their initial platelet counts. Three groups of rats were injected intravenously once daily for seven days with 0.1, 10, and 1000 μg/kg of the C-terminally truncated tyrosyl-tRNA synthetase polypeptide, respectively. Three additional groups were administered with the same dosages of Y341A. One control group received a daily injection of buffer only (0.5×PBS, 2 mM DTT) and an additional control group was injected daily with 90 μg/kg of thrombopoietin (R&D Systems, Minneapolis, Minn.).

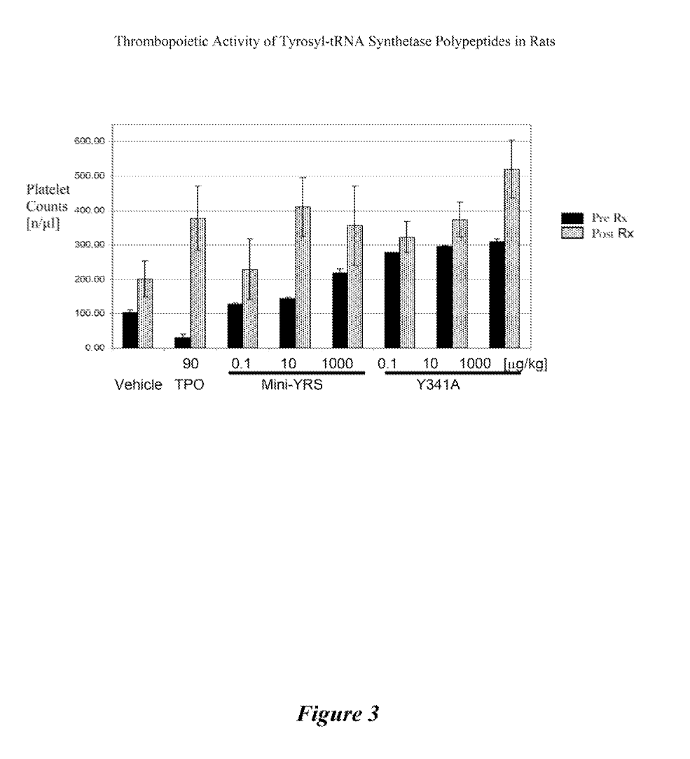

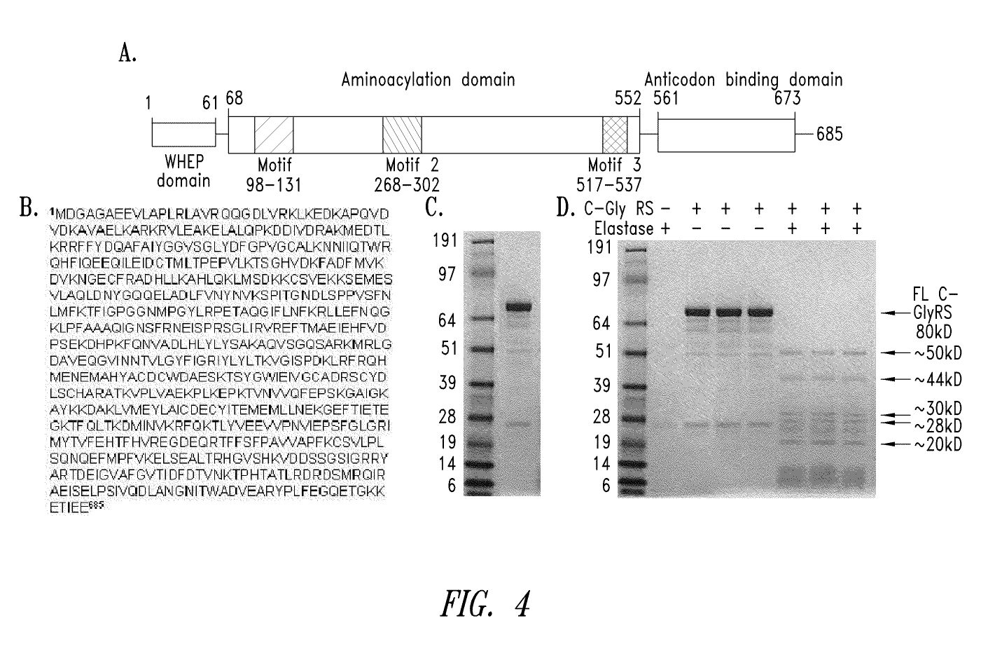

Administration of the two tyrosyl-tRNA synthetase polypeptides resulted in a marked elevation in platelet counts, comparable or superior to that observed in the thrombopoietin group (See FIG. 3). These results show that tyrosyl-tRNA synthetase polypeptides are capable of stimulating thrombopoiesis in vivo.

### Example 5

### Tyrosyl-tRNA Synthetase Polypeptides are Chemoattractants for Megakaryocytes

MO7e cells (DSMZ, Braunschweig, Germany) were cultured in RPMI-1640 medium supplemented with 20% heat-inactivated FBS and 10 ng/ml IL-3 (R&D Systems, Minneapolis, Minn.). Cells were maintained at a density of 2×10⁵ to 1×10⁶/ml and RPMI-1640 medium with 0.1% BSA was used as migration buffer. Before the migration assay, cells were serum-starved for 30 minutes in migration buffer and loaded with 8 μg/ml calcein AM (Invitrogen, Carlsbad, Calif.). Cells were spun down at 200 g for 5 minutes without brake and washed once with migration buffer to remove free calcein AM. Cell density was adjusted to 1×10⁷/ml and 100 μl were added to 6.5 mm transwell 8.0 μm pore filter inserts (Costar, Cambridge, Mass.). 600 μl migration buffer containing either PBS, a control chemokine, or the tyrosyl-tRNA synthetase polypeptides were added to the lower chamber and cells were allowed to migrate for 4 to 16 hours (for the 16-hour migration time, cells were stained after migration). Cells that migrated to the lower chamber were collected and resuspended in 100 μl PBS, transferred into 384-well opaque Greiner plate and counted by fluorescence in a plate reader. Tyrosyl-tRNA synthetase polypeptides stimulated migration of MO7e megakaryoblasts.

### Example 6

### Tyrosyl-tRNA Synthetase Polypeptides Promote Cell Adhesion to Endothelial Monolayers

The ability of YRS polypeptides to stimulate adhesion of THP-1 cells to endothelial monolayers of HUVEC-2 cells was tested. HUVEC-2 cells (BD Biosciences, San Jose, Calif.) were cultured in EGM-2 medium (Lonza, Allendale, N.J.) and used before they reached 10 passages. THP-1 cells (ATCC, Manassas, Va.) were cultured in RPMI-1640 medium supplemented with 10% heat-inactivated FBS and maintained at a density of 2−4×10⁵/ml. Cells were seeded at approximately 1×10⁴ cells/well into fibronectin-coated (10 μg/ml, 2 hours at 37° C.), opaque 96-well plates.

HUVEC-2 cells were grown until a monolayer was formed and then stimulated overnight in EGM-2 medium with either PBS, IL-1 or the tyrosyl-tRNA synthetase polypeptides. THP-1 cells were collected and incubated for 30 minutes in RPMI-1640 serum-free medium containing 0.1% BSA and calcein AM (6 μl/ml). The cells were then washed in RPMI-1640 serum-free medium containing 0.1% BSA and resuspended at a density of 1.5×10⁵ cells/ml in RPMI medium containing 10% FBS. 100 μl THP-cells were added to the HUVEC monolayer and incubated for 15 minutes. Unbound THP-1 cells were washed with PBS twice and the remaining cells were fixed with 2% formaldehyde and counted by fluorescence in a plate reader.

Adhesion molecule expression in endothelial monolayers was measured following exposure to tyrosyl-tRNA synthetase polypeptides. 1×10⁴ HUVEC-2 cells were seeded into a 96-well plate and grown for 48 hours as described in the previous paragraph. Tyrosyl-tRNA synthetase polypeptides, diluted in growth media, were added to the wells and incubated for 16 hours. The culture medium was removed and cells were fixed with 50 μl of Z fix (Anatech Ltd, Battle Creek, Mich.) for 15 minutes at room temperature. Wells were subsequently blocked with 50 μl of casein for 1 hour followed by multiple 200 μl washes with PBS. All subsequent reagents were diluted in casein and all steps were performed at room temperature. Antibodies directed against VCAM-1 and E-selectin (Santa Cruz Biotech, Santa Cruz, Calif.) were added for 1 hour. Wells were then washed as above and an HRP-labeled secondary antibody (Jackson Immunoresearch, West Grove, Pa.) was added for 1 hour. Wells were washed and the substrate for HRP was added. 15 minutes later, an equal volume of 2 M sulfuric acid was added and absorbance determined at 450 nm. VCAM-1 expression was increased following stimulation of the endothelial cells with tyrosyl-tRNA synthetase polypeptides.

### Example 7

### Tyrosyl-tRNA Synthetase Polypeptides Stimulate Migration of 293 and CHO Cell Lines Transfected with the CXCR-2 Receptor

The effects of tyrosyl-tRNA synthetase polypeptides on CXCR-2 signaling was tested by measuring the migration of CXCR-2 expressing cells in response to said polypeptides. 293/CXCR-2 cells were maintained in DMEM medium supplemented with 10% heat-inactivated FBS, 1% Penicillin-Streptomycin and 800 μg/ml Geneticin, all purchased from Invitrogen, Carlsbad, Calif. DMEM medium with 0.1% BSA was used as migration buffer. Prior to migration assay, cells were serum-starved for 30 minutes in migration buffer, centrifuged at 200 g for 5 minutes and resuspended in migration buffer at a final density of 1×10⁶ cells/ml. 100 μl were added to 6.5 mm transwell filter inserts (Costar, Cambridge, Mass.) and 600 μl migration buffer containing a control chemokine, the tyrosyl-tRNA synthetase polypeptides or buffer only were added to the plate lower chambers. Cells were allowed to migrate for 4 hours and the remaining cells in the upper chamber (transwell filter inserts) were removed with a cotton swap. The filter inserts were then transferred to a new 24-well plate containing 500 μl cell dissociation buffer (Invitrogen, Carlsbad, Calif.) and 12 μg/ml Calcein AM (Invitrogen, Carlsbad, Calif.). After 1 hour incubation at 37° C., cells were collected and resuspended in 100 μl PBS, transferred into a 384-well opaque Greiner plate, and counted by fluorescence in a plate reader.

CHO-K1/CXCR-2 cells were maintained in F12 medium supplemented with 10% heat-inactivated FBS, 1% Penicillin-Streptomycin-Glutamine and 800 μg/ml Geneticin. F12 medium with 0.5% BSA was used as migration buffer. Prior to migration, cells were serum-starved for 30 minutes in migration buffer, collected by using cell dissociation buffer, spun down at 200 g for 5 minutes and resuspended in migration buffer at the final density of 1×10⁶ cells/ml. 100 μl were added to 6.5 mm transwell filter inserts and 600 μl migration buffer containing a control chemokine, the tyrosyl-tRNA synthetase polypeptides or buffer only were added to the plate lower chambers. Cells were allowed to migrate for 3 hours and the remaining cells in the upper chamber (transwell filter inserts) were removed with a cotton swap. The filter inserts were then transferred to a new 24-well plate containing 500 μl PBS and 12 μg/ml Calcein AM. After 30 minutes incubation at 37° C., filters were transferred again into a new 24-well plate containing 500 μl phenol/red-free trypsin. After 2 to 5 minutes incubation, detached cells were collected and resuspended in 100 μl PBS, transferred into a 384 well opaque Greiner plate and counted by fluorescence in a plate reader. Tyrosyl-tRNA synthetase polypeptides induced migration of CXCR-2 transfected cells.

### Example 8

### Tyrosyl-tRNA Synthetase Polypeptides Stimulate Polymorphonuclear(PMN) Cell Migration

To test the effects of YRS polypeptides on PMN cell migration, human granulocyte cells were purified from fresh human peripheral blood using RosetteSep® Human Granulocyte Enrichment Kit (StemCell Technologies, Vancouver, BC) according to the manufacturer's instructions. Serum-free RPMI medium supplemented with 0.5% FBS was used as migration buffer. 4×10⁷ cells were resuspended in 1 ml migration buffer and incubated for 30 minutes with 8 μl of a 1 mg/ml Calcein AM solution (Invitrogen, Carlsbad, Calif.). Cells were collected, spun down at 200 g for 5 minutes without brake, washed once with migration buffer and resuspended in the same buffer at a final density of 1×10⁷/ml.

100 μl were added to 6.5 mm transwell filter inserts (Costar, Cambridge, Mass.) and 600 μl migration buffer containing a control chemokine, the tyrosyl-tRNA synthetase polypeptides or buffer only were added to the plate lower chambers. Cells were allowed to migrate for 45 minutes in the incubator and cells that migrated to the lower chamber were collected, resuspended in 100 μl PBS, transferred into a 384-well opaque Greiner plate and counted by fluorescence in a plate reader.

The tyrosyl-tRNA synthetase polypeptides induced a biphasic migration of PMN both at low pM and at higher μM concentrations, as illustrated by a bell-shaped migration curve typically observed with chemokines.

### Example 9

### Generation and Identification of Human Glycyl-tRNA Synthetase (GlyRS) Proteolytic Fragments

Full-length recombinant human GlyRS having an amino acid sequence as set forth in SEQ ID NO:16 was expressed and purified from E. coli using nickel IMAC chromatography. To generate fragments of GlyRS by controlled proteolysis, the full-length protein was treated with 167 nM human neutrophil elastase for 30 minutes before separation of the fragments by SDS-PAGE. Fragments generated by cleavage with neutrophil elastase were analyzed using LC/MS/MS to determine accurate masses for each fragment. In addition individual fragments were excised from an SDS-PAGE gel and subjected to in-gel trypsin digestion followed by LC/MS/MS analysis to identify the portion of the full-length protein from which the fragment was generated and to identify non-trypsin cleavage sites that could be attributed to neutrophil elastase.

The identity of these peptide boundaries is summarized in Table 1; residues in bold are non-trypsin cleavage sites indicating that the exact cleavage site of elastase (thus exact N- or C-terminus) of that fragment has been identified.

TABLE 1
GlyRS proteolytic fragments

| | Whole | | | | |
|---|---|---|---|---|---|
| | mass | Protease | N-term. | C-term. | Non-tryptic |
| | (Da) | used | boundary | boundary | peptide found |
| 1 | 71384 | No | A57 | E685 | |
| | | protease | | | |
| 2 | 50782 | No | P239 | E685 | PGYLRPETA |
| | | protease | | | QGIFLNFK |
| | | | | | (SEQ ID NO: 18) |
| 3 | 53406 | elastase | T214 | E685 | TGNDLSPP |
| | | | | | VSFNLMFK |
| | | | | | (SEQ ID NO: 19) |
| 4 | 41000- | elastase | F311- | E685 | |
| | 43000 | | L338 | | |
| 5 | 28096 | elastase | N439 | E685 | 439NVVQFEPSK |
| | | | | | (SEQ ID NO: 20) |
| 6 | 25328 | elastase | T214 | V438 | TGNDLSPPVSFNLMFK |
| | | | | | (SEQ ID NO: 19) |
| | | | | | 439NVVQFEPSK |
| | | | | | (SEQ ID NO: 20) |
| 7 | 22398 | elastase | T214 | R420 | TGNDLSPPVSFNLMFK |
| | | | | | (SEQ ID NO: 19) |
| 8 | 19783 | elastase | L511 | E685 | LYVEEVVPNV |
| | | | | | IEPSFGLGR |
| | | | | | (SEQ ID NO: 21) |
| 9 | | elastase | T214 | 325- | TGNDLSPPVSFNLMFK |
| | | | | 338 | (SEQ ID NO: 19) |
| 10 | 4841 | elastase | A85 | T127 | AIYGGVSGLY |
| | | | | | DFGPVGCALK |
| | | | | | (SEQ ID NO: 22) |
| | | | | | QHFIQEEQILEIDCT |
| | | | | | (SEQ ID NO: 23) |
| 11 | 3675 | elastase | R25 | 156 | |

FIG. 4 (A-D) shows the domain structure and amino acid sequence of GlyRS, and illustrates the SDS-PAGE separation of fragments of GlyRS generated by controlled proteolysis of the full-length GlyRS protein with human neutrophil elastase. As described below, certain of these fragments were tested for the hematopoietic-modulating activities. Included are variants and biologically active fragments of the above GlyRS polypeptides.

### Example 10

### Generation and Identification of Endogenous Human Glutaminyl-tRNA Synthetase (QRS) Fragments

Full-length recombinant human QRS (SEQ ID NO:25) was expressed and purified from E. coli using nickel IMAC chromatography. Endogenous proteolytic fragments were generated, purified, and subsequently characterized using LC/MS/MS. Without wishing to be bound by any one theory, it is believed that these fragments are indicative of those that would be created in human cells through the process of natural proteolysis.

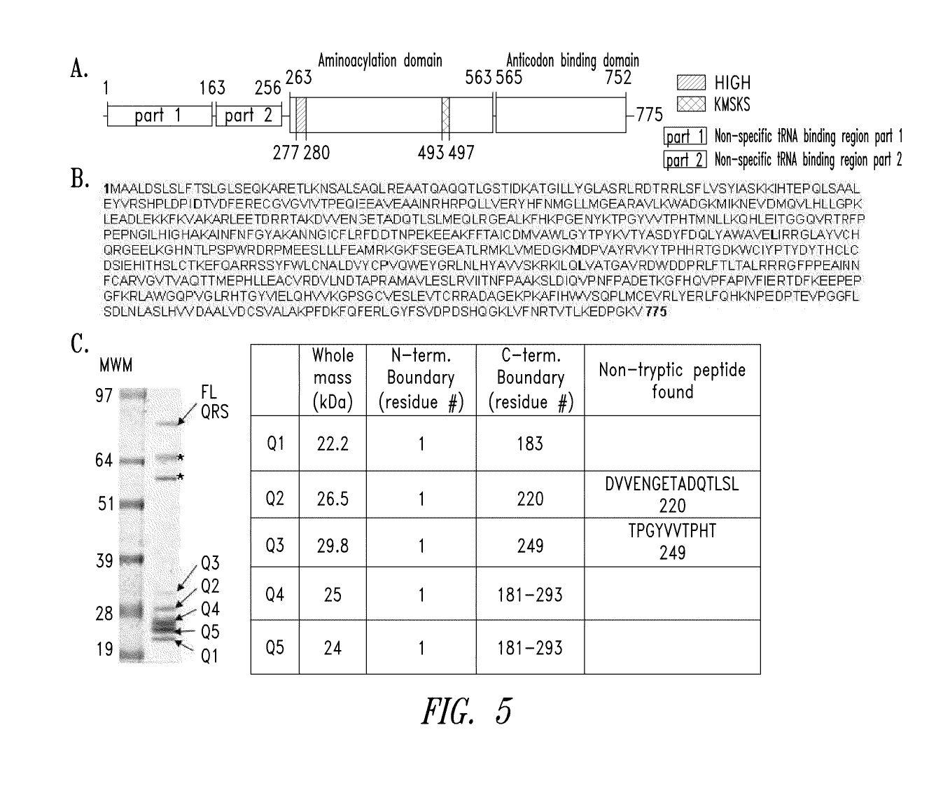

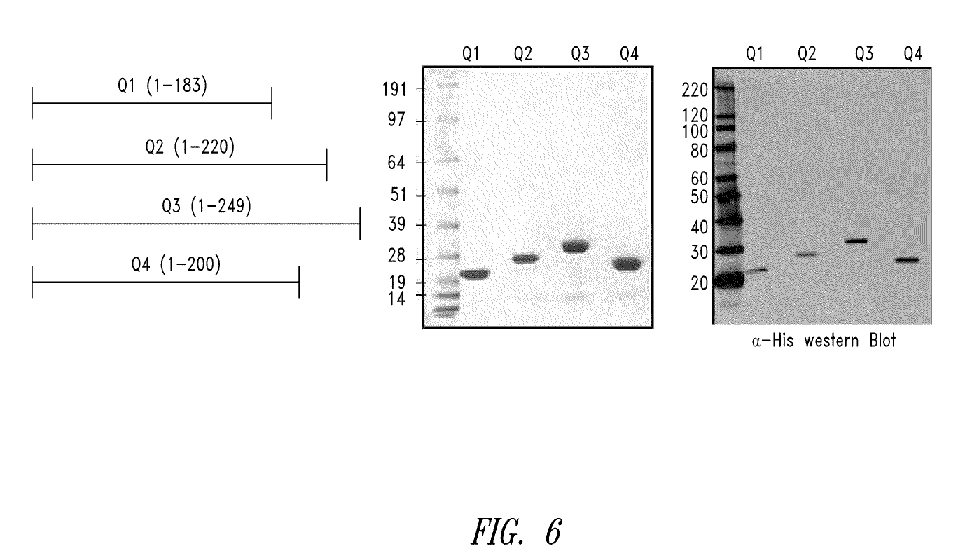

To identify the residues at which proteolysis occurs for human QRS, the proteins were separated by SDS-PAGE run in 4-12% MOPS, gel slices containing the fragments were excised and subjected to in-gel trypsin digestion followed LC/MS/MS analysis. This process allowed the identification of both the portion of the full-length protein from which the fragments were generated and the non-trypsin cleavage sites that could be attributed to endogenous proteolytic cleavage. All protein fragments identified represented the N-terminal portion of QRS. See Table 2 below, and FIG. 5 (A-D).

TABLE 2
Endogenous QRS proteolytic fragments

| | Whole | | | |
|---|---|---|---|---|
| | mass | N-term. | C-term. | Non-tryptic |
| | (DA) | boundary | boundary | peptide found |
| Q1 | 22200 | 1 | 183 | |
| Q2 | 26500 | 1 | 220 | DVVENGETADQTLSL220 |
| | | | | (SEQ ID NO: 26) |
| Q3 | 29800 | 1 | 249 | TPGYVVTPHT249 |
| | | | | (SEQ ID NO: 27) |
| Q4 | 25000 | 1 | 181-293 | |
| | | | (200) | |
| Q5 | 24000 | 1 | 181-293 | 439NVVQFEPSK |
| | | | | (SEQ ID NO: 20) |

QRS fragments closely matching those identified by LC/MS/MS in Table 2 above were cloned into an E. coli protein expression vector for overexpression and purification. Proteins were purified using Nickel IMAC chromatography and contaminants were removed using a Sartobind Q membrane (Sartorius). See FIG. 6.

### Example 11

### Aminoacyl-tRNA Synthetase Polypeptides Impact Megakaryocyte Progenitor Cells in Bone Marrow Cell Cultures

To test the effects of YRS polypeptides on megakaryocyte progenitor cells in bone marrow cell cultures, clonogenic progenitors of the megakaryocyte (CFU-Mk; Colony Forming Unit-Megakaryocyte) lineage were assessed in serum-free, collagen-based media MegaCult-C® 4950 supplemented with proprietary concentrations of cytokines (StemCell Technologies, Vancouver, BC). Normal human bone marrow light density cells (Lonza, Allendale, N.J.) were stored at −152° C. until required for the assay. On the day of the experiment, cells were thawed rapidly at 37° C., the contents of the vial were diluted in 10 mL of Iscove's modified Dulbecco's medium (IMDM) containing 2% fetal bovine serum (FBS) and washed by centrifugation (1200 rpm for 10 minutes, room temperature). The supernatant was discarded and the cell pellet resuspended in a known volume of IMDM containing 2% FBS. A cell count (3% glacial acetic acid) and viability assessment (trypan blue exclusion test) were performed.

The aminoacyl-tRNA synthetase polypeptides (stored in 50% glycerol/0.5×PBS/2 mM DTT) were dialyzed in 0.5×PBS/2 mM DTT for a total of 5 hours, with one change of buffer after 3 hours in order to remove glycerol. After dialysis, proteins and buffer sample were sterile filtered and concentration was adjusted to compensate for the increase in volume.

Test proteins (AARS polypeptides) were added to tubes of serum-free, collagen-based media MegaCult-C® 4950 supplemented with cytokines (rhTpo, rhIL-3, and rhIL-6). Standard control cultures (containing no test protein) and solvent control cultures (containing no test protein but equivalent concentrations of buffer) were also initiated. Bone marrow cells were then added to each tube of media to give a final concentration of 1×10⁵ cells per slide. Bovine collagen was then added, tubes were vortexed, and contents dispensed into triplicate double chamber slides. All cultures were incubated for 10-12 days at 37° C., 5% CO₂.

Following incubation, cultures were assessed microscopically for colony formation prior to dehydration and fixation of the slide. Using an antibody staining protocol to detect GPIIa/IIIb (CD41) expression, the colonies on the slide were stained using an alkaline phosphatase detection system as described in the StemCell Technical Manual, “Assays for the Quantitation of Human and Murine Megakaryocytic Progenitors”, Section 7, herein incorporated by reference in its entirety. Colony numbers were scored and assessed by trained StemCell personnel. The colonies were divided into the following categories, based on size and morphology: i) CFU-Mk (2-20)—the small megakaryocytic colony derived from this more mature progenitor cell contains 2-20 cells; ii) CFU-Mk—the medium megakaryocytic colony derived from this more primitive progenitor cell contains 21-49 cells and; iii) CFU-Mk (>50)—the large megakaryocytic colony derived from this most primitive lineage-restricted progenitor cell contains >50 cells.

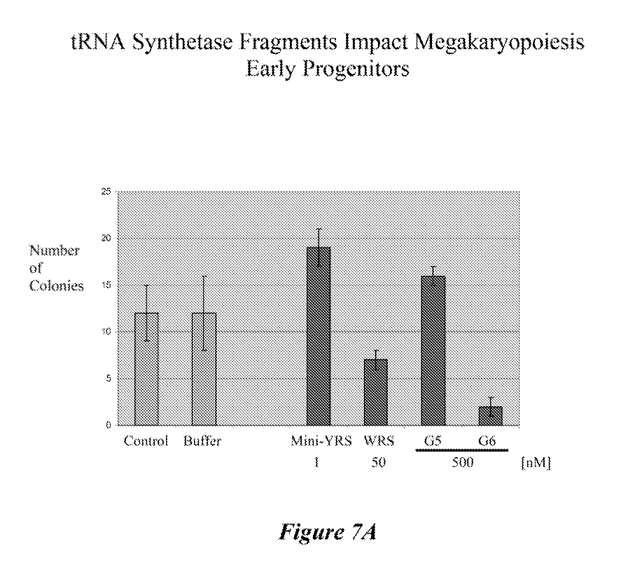

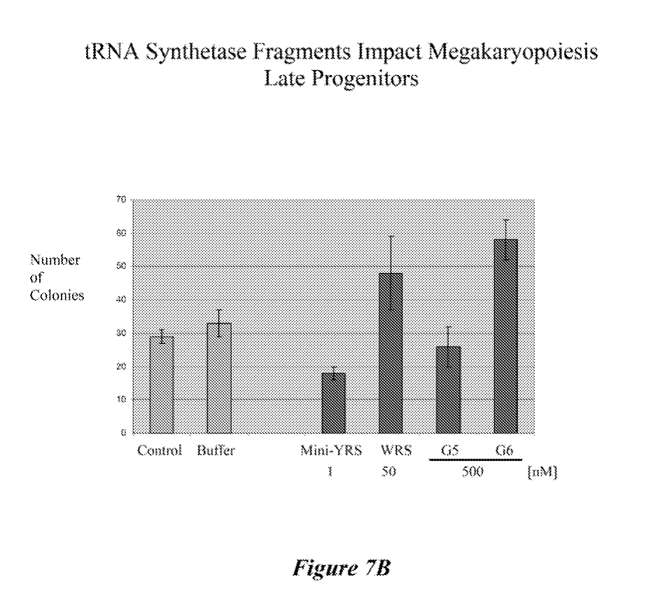

FIG. 7 shows the impact of a variety of AARS polypeptide on the most primitive lineage-restricted (early) progenitors (FIG. 7(A)), and on the more mature progenitors (FIG. 7(B)). Mini-YRS and the G5 fragment of GlyRS (amino acids 439-685 of human GlyRS) increase the number of early progenitors and decrease the number of late progenitors. A WRS fragment (see, e.g., SEQ ID NOS:34 and 35) and the G6 fragment of GlyRS (amino acids 214-438 of human GlyRS) decrease the number of early progenitors and increase the number of late progenitors.

### Example 12

### Aminoacyl-tRNA Synthetase Polypeptides Impact Erythrocyte Progenitor Cell Formation in Bone Marrow Cultures

To test the effects of AARS polypeptides on erythrocyte progenitor cells in bone marrow cultures, clonogenic progenitors of the erythroid (CFU-E; Colony Forming Unit-Erythrocyte) lineage were assessed in the methylcellulose-based medium MethoCult® 84434 (StemCell Technologies, Vancouver, BC). Normal human bone marrow light density cells (Lonza, Allendale, N.J.; lot #07B21195) were stored at −152° C. until required for the assay. On the day of the experiment, the cells were thawed rapidly at 37° C., the contents of the vial were diluted in 10 mL of Iscove's modified Dulbecco's medium (IMDM) containing 2% fetal bovine serum (FBS) and washed by centrifugation (1200 rpm for 10 minutes, room temperature). The supernatant was discarded and the cell pellet resuspended in a known volume of IMDM containing 2% FBS. A cell count (3% glacial acetic acid) and viability assessment (trypan blue exclusion test) were performed.

The AARS polypeptides (stored in 50% glycerol/0.5×PBS/2 mM DTT) were dialyzed in 0.5×PBS/2 mM DTT for a total of 5 hours, with one change of buffer after 3 hours in order to remove glycerol. After dialysis, proteins and buffer sample were sterile filtered and concentration was adjusted to compensate for the changes in volume. Dialyzed proteins were added directly into the methylcellulose media to achieve the final test concentrations.

Test proteins (tRNA synthetase polypeptides) were added to tubes of methylcellulose-based media MethoCult® 84434, which contains optimal proprietary concentrations of cytokines (StemCell Technologies, Vancouver, BC). Standard control cultures (containing no test protein) and solvent control cultures (containing no test protein but equivalent concentrations of dialyzed 50% glycerol/0.5×PBS/2 mM DTT buffer) were also initiated. Bone marrow cells were then added to each tube of media and cultures plated in triplicate at 1×10⁴ cells per dish. Following 14 days in culture at 37° C., 5% CO₂, the colonies were assessed and scored by trained StemCell personnel.

The mean+/−1 standard deviation was calculated for triplicate cultures. Standard t-tests were performed to assess if there was a difference in the number of colonies generated between solvent control and treated cultures. Due to the potential subjectivity of colony enumeration, a p-value of less than 0.01 is deemed significant. Photographs were taken of representative erythroid progenitor derived colonies from solvent controls and various test protein concentrations illustrating normal colonies and colonies where the growth was impacted due to the activity of the test proteins.

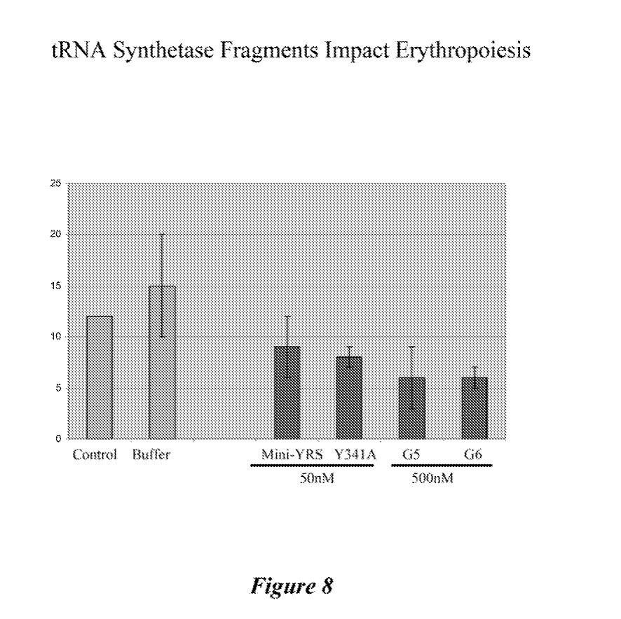

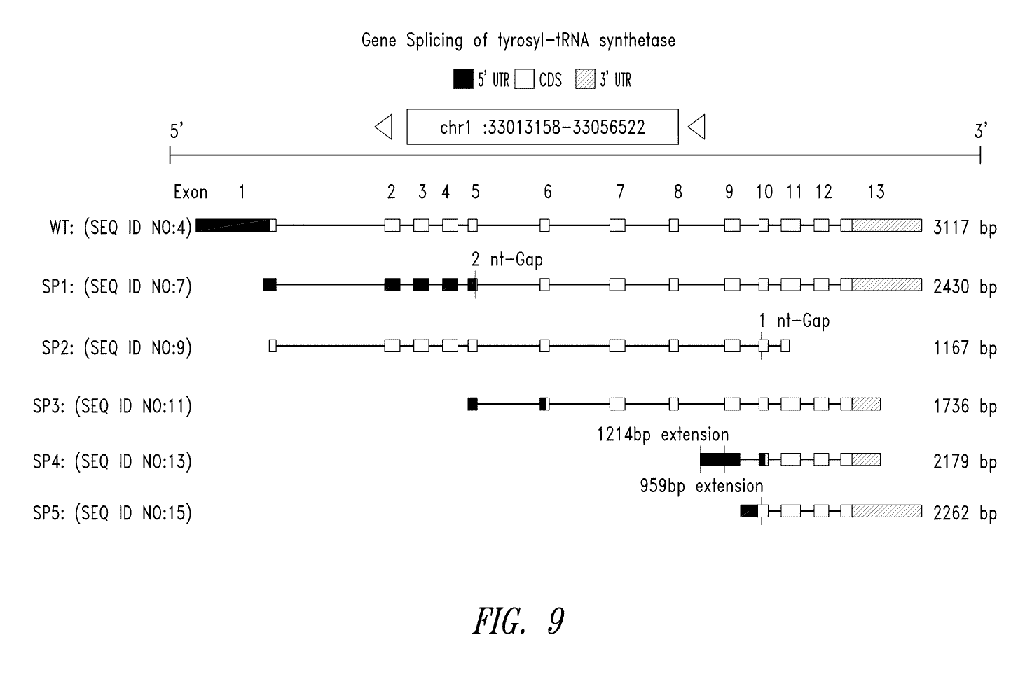

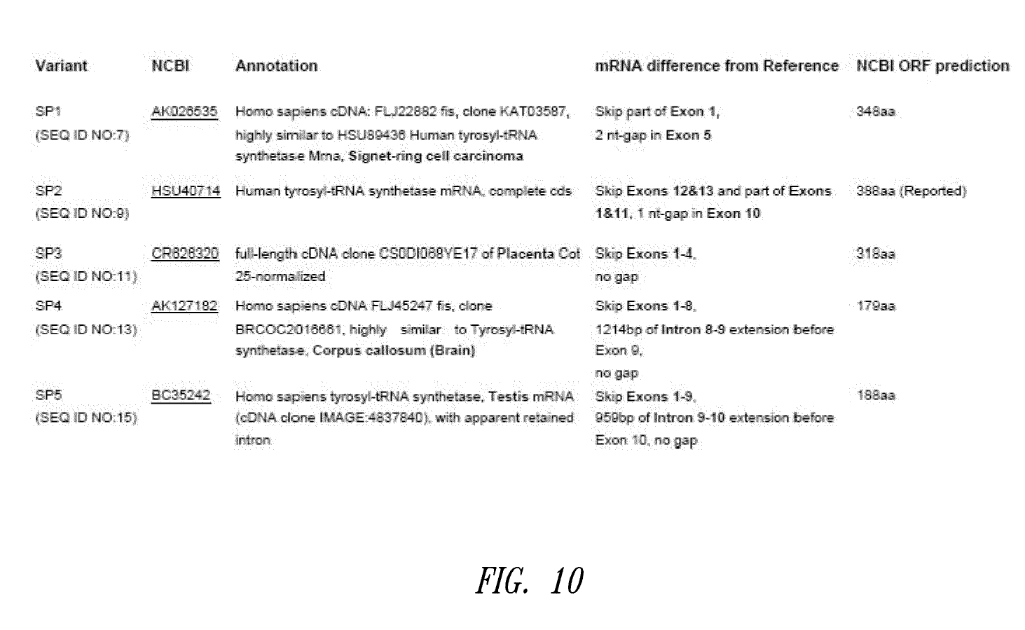

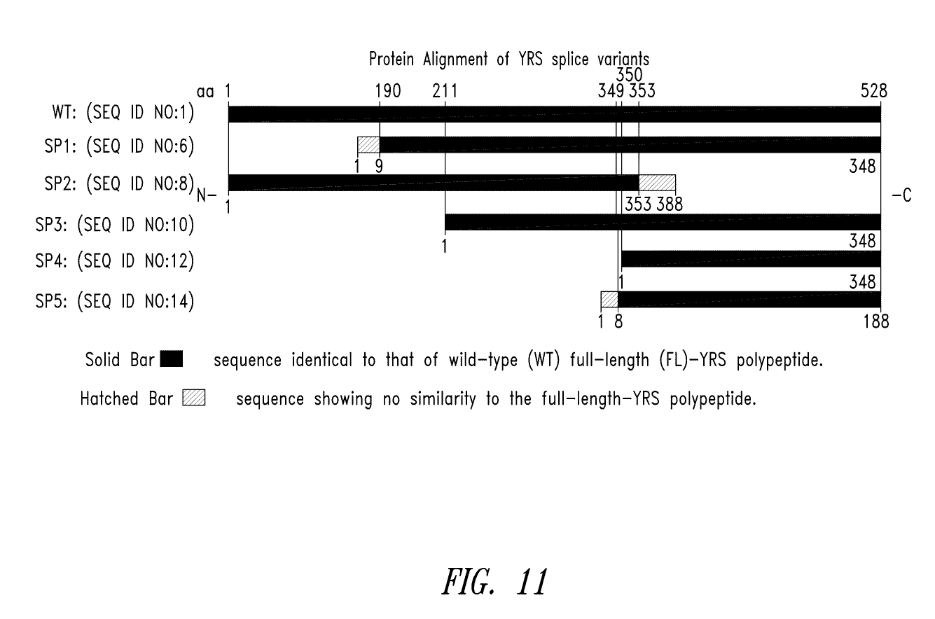

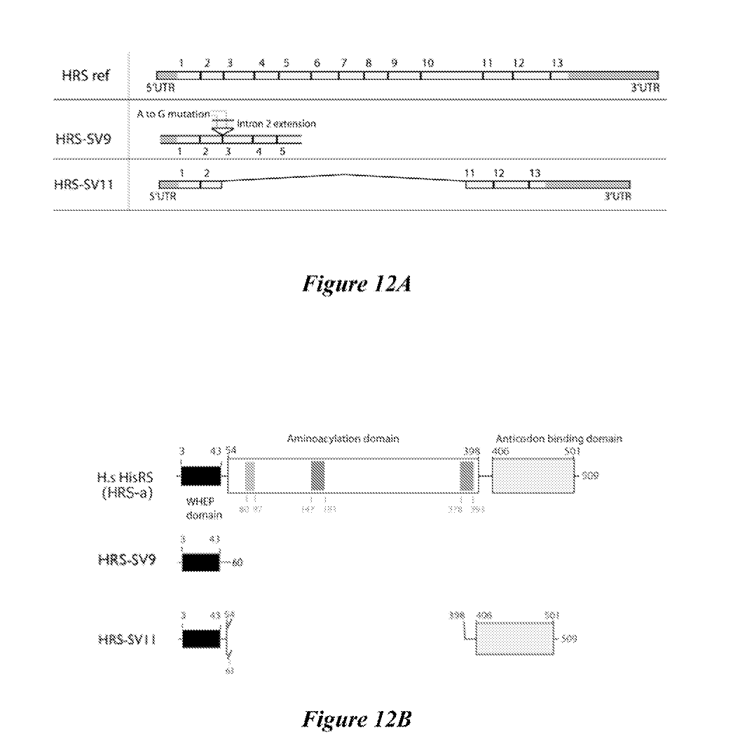

FIG. 8 shows the impact of a variety of AARS polypeptides on erythropoiesis, as illustrated by the reduced formation of erythroid progenitor cells. G5 and G6 are active fragments of human GlyRS (amino acids 439-685 and amino acids amino acids 214-438, respectively). The y-axis represents the number of colonies (CFU-E).

### Example 13

### Tyrosyl-tRNA Synthetase Polypeptides Increase Platelet Counts by Mechanisms Distinct from Those of Thrombopoietin

Thpo^{-/-} and/or Mpl^{-/-} mice are injected intravenously with recombinant tyrosyl-tRNA synthetase polypeptides, daily, for 7 days. Included are tyrosyl-tRNA synthetase polypeptides having a Y341A mutation, and mini-YRS. Blood is collected by retro-orbital bleeding using capillary pipettes and platelet levels are determined using a blood analyzer or a hematocytometer. Thpo^{-/-} and/or Mpl^{-/-} mice have typically 85% fewer platelets than normal mice.

In this Example, platelets counts are increased and/or restored to normal or near normal levels after administration of tyrosyl-tRNA synthetase polypeptides demonstrating that these polypeptides exert their thromobopoietic effects by thrombopoietin-independent and/or thrombopoietin receptor-independent mechanisms.

### Example 14

### Tyrosyl-tRNA Synthetase Polypeptides Impact Cells Present in the Bone Marrow

Thpo^{-/-} and/or Mpl^{-/-} and/or normal mice are injected intravenously with recombinant tyrosyl-tRNA synthetase polypeptides (e.g., Y341A, mini-YRS), daily, for 7 days. Blood is collected by retro-orbital bleeding using capillary pipettes and platelet levels are determined using a blood analyzer or a hematocytometer. Bone marrow samples are prepared to determine the effects of the tyrosyl-tRNA synthetase polypeptides.

In this Example, administration of the tyrosyl-tRNA synthetase polypeptides results: i) in a change in the number and/or activity of neutrophils and, in turn, this results in a change in the distribution of stem cells in the bone marrow (e.g., an increase in the number of neutrophils and/or activation of the neutrophils resulting in the redistribution or mobilization of stem cells to a more permissive environment where they can proliferate and differentiate); ii) in a change in the number and/or activity of hematopoietic progenitor/precursor cells that, ultimately, results in an increase in platelet counts; iii) in a change in the development, cell cycle, proliferation, distribution, mobilization, differentiation, migration, attachment, cell-cell interactions, maturation, endomitosis, and/or polyploidy of megakaryocytes precursors and/or megakaryocytes; iv) in a change (e.g., stimulation) in the transition from pro-platelet to platelets and in their release in the bone marrow blood vessels; and/or v) in a change in platelet progeny. In this Example, some or all of the effects of the tyrosyl-tRNA synthetase polypeptides are discovered using in vitro assays.

### Example 15

### Tyrosyl-tRNA Synthetase Polypeptides Impact Bone Marrow Environment and/or Vasculature

Normal mice are treated with intravenous injection of 5-fluorouracil, sublethal irradiation or lethal irradiation (either alone or followed by bone marrow transplant) to study hematopoietic maintenance and recovery/regeneration after myelosuppression. Recombinant tyrosyl-tRNA synthetase polypeptides are administered to mice either prophylactically or after myelosuppression and their impact on the maintenance, homeostasis and/or regeneration of the bone marrow environment, osteoblastic niche, stem cell niche, and vasculature is determined using histology staining, immunohistochemistry, fluorescent microscopy, enzyme and reporter gene activity, imaging, flow cytometry and cell enumeration techniques. In this Example, the tyrosyl-tRNA synthetase polypeptides demonstrate an impact on bone marrow structures and/or cells leading to an increase in platelet counts (e.g., stimulation of blood vessel regeneration after myelosuppression, other changes in the marrow microenvironment [osteoblastic, stem cell, vasculature niches] promoting interactions between the vasculature, or other niches, and hematopoietic stem and progenitor cells, resulting in the stimulation of hematopoiesis and/or contributing to homeostasis).

### Example 16

### Tyrosyl-tRNA Synthetase Polypeptides Improve Chemotherapy- and/or Radiation-Induced Thrombocytopenia

Normal mice are treated with intravenous injection of 5-fluorouracil, sublethal irradiation or lethal irradiation (either alone or followed by bone marrow transplant) to recapitulate thrombocytopenia observed in patients receiving chemotherapy and radiation treatment. Recombinant tyrosyl-tRNA synthetase polypeptides are administered to mice either prophylactically or after myelosuppression and their impact on thrombocytopenia is determined using the techniques of Example 13-15. In this Example, administration of tyrosyl-tRNA synthetase polypeptides results in an increase in platelet counts and demonstrates their therapeutic potential in the treatment of thrombocytopenia in cancer patients. The methods and reagents described in Example 15 are used to discover and define the biological effect of the tyrosyl-tRNA synthetase polypeptides and their mechanism of action in platelet count recovery, as well as the protective effects enabling enhanced/optimized chemotherapy/radiation regimens in the management of cancer patients.

In this Example, chemotherapy and/or radiation treatment results in bone marrow damages that mimic or recapitulate (aspects of) human diseases characterized by marrow failures (e.g., myelodysplatic syndromes, cytopenia, etc.). The tyrosyl-tRNA synthetase polypeptides or tryptophanyl-tRNA synthetase polypeptides are administered to the animals and any beneficial effect on the bone marrow environment and/or on the production of blood cells that has potential therapeutic benefit in the treatment of (aspects of) bone marrow failures is examined/determined using methods and techniques similar to those described in Example 15.

### Example 17

### Combination Treatment/Therapy with Tyrosyl-tRNA Synthetase Polypeptides

Methods and techniques described in Examples 13-16 are used to determine whether synergism, additivity or enhanced therapeutic benefits can be derived from combining tyrosyl-tRNA synthetase polypeptides with other molecules involved in hematopoiesis, including, but not limited to, thrombopoietin, thrombopoietin receptor agonists and mimetics, molecules binding to the Mpl receptor, IL-11 and other interleukins, SDF-1, FGF-4 and other members of the FGF family, VEGF and other angiogenic molecules, ligands binding to CXCR-1, CXCR-2, CXCR-4 and other chemokine receptors involved in hematopoiesis, and/or adhesion molecules.

### Example 18

### Other tRNA Synthetase Polypeptides

Methods and techniques described in Examples 13-17 are used to identify other tRNA synthetase polypetides and molecules with similar effects, as well as other tRNA synthetase polypetides and molecules that oppose or modulate the effects of the tyrosyl-tRNA synthetase polypetides. For instance, tryptophanyl-tRNA synthetase polypetides or glycyl-tRNA synthetase polypetides are used in the above Examples to characterize their angiostatic properties and their potential effects in reducing, inhibiting or modulating hematopoiesis. In certain instances, tyrosyl-tRNA synthetase polypeptides are shown to have the opposite effects described above for tyrosyl-tRNA synthetase polypeptides.

### Example 19

### Tyrosyl-tRNA Synthetase Polypeptides Impact Platelet Progeny

Platelets are isolated from human blood by mixing 45 ml of fresh blood with 5 ml 2% EDTA followed by centrifugation at 200 g for 10 minutes at room temperature. The platelet-rich phase is transferred to a new tube and centrifuged at 1500 g for 5 minutes. The pellet is washed twice with 30 ml of reagent I and II in a 49:1 ratio (Reagent I: NaCl 8.1 g, KCl 0.4 g, Tris 1.83 g in 1000 ml water, pH 7.4; Reagent II: 2% EDTA in 0.7% NaCl, pH 7.4). Washed platelets are resuspended at a density of 100,000/1 in serum-free M199 medium and cultured at 37° C. under gentle rotation. Platelets are counted using a blood analyzer or a hematocytometer before and after treatment with tyrosyl-tRNA synthetase polypeptides.

In this Example, the addition of tyrosyl-tRNA synthetase polypeptides directly to the culture medium results in an increase in the number of functional platelets generated by the production of new cell bodies indistinguishable from the parent platelets. Standard molecular biology methods are used to determine whether the tyrosyl-tRNA synthetase polypeptides can also confer protection from cell death and extend the life of platelets.

1. A composition for modulating hematopoiesis, comprising an isolated aminoacyl-tRNA synthetase (AARS) polypeptide, or a biologically active fragment or variant thereof, wherein the polypeptide modulates hematopoiesis, and a pharmaceutically acceptable excipient or carrier.

2. The composition of claim 1, wherein the AARS polypeptide is a tyrosyl-tRNA synthetase (YRS), a tryptophanyl-tRNA synthetase (WRS), a glutaminyl-tRNA synthetase (QRS), a glycyl-tRNA synthetase (GlyRS), a histidyl-tRNA synthetase (HisRS), a seryl-tRNA synthetase (SRS), a phenylalanyl-tRNA synthetase (PheRS), an alanyl-tRNA synthetase (AlaRS), an asparaginyl-tRNA synthetase (AsnRS), an aspartyl-tRNA synthetase (AspRS), a cysteinyl-tRNA synthetase (CysRS), a glutamyl-tRNA synthetase (ERS), a prolyl-tRNA synthetase (ProRS), an arginyl-tRNA synthetase (RRS), an isoleucyl-tRNA synthetase (IRS), a leucyl-tRNA synthetase (LRS), a lysyl-tRNA synthetase (KRS), a threonyl-tRNA synthetase (TRS), a methionyl-tRNA synthetases (MRS), or a valyl-tRNA synthetase (VRS).

3. The composition of claim 2, comprising a proteolytic fragment of the AARS polypeptide.

4. The composition of claim 3, wherein the sequence of the proteolytic fragment is derived by incubating the polypeptide with a protease in vitro.

5. The composition of claim 3, wherein the sequence of the proteolytic fragment is derived by recombinantly expressing the AARS polypeptide in a cell, wherein the cell comprises one or more recombinant or endogenous proteases.

6. The composition of claim 3, wherein the proteolytic fragment comprises the sequence of an endogenous, naturally-occurring human or mouse AARS proteolytic fragment.

7. The composition of claim 2, wherein the aminoacyl-tRNA synthetase is a YRS polypeptide.

8. The composition of claim 7, wherein the YRS polypeptide is truncated at its C-terminus.

9. The composition of claim 7, wherein the YRS polypeptide comprises the amino acid sequence of SEQ ID NO: 1, 2, 3, 6, 8, 10, 12, or 14, wherein at least about 1-50 amino acid residues are truncated from its C-terminus.

10. The composition of claim 7, wherein the YRS polypeptide comprises the amino acid sequence of SEQ ID NO: 1, 2, 3, 6, 8, 10, 12, or 14, wherein at least about 50-100 amino acid residues are truncated from its C-terminus.

11. The composition of claim 7, wherein the YRS polypeptide comprises the amino acid sequence of SEQ ID NO: 1, 2, 3, 6, 8, 10, 12, or 14, wherein at least about 100-150 amino acid residues are truncated from its C-terminus.

12. The composition of claim 7, wherein the YRS polypeptide comprises the amino acid sequence of SEQ ID NO: 1, 2, 3, 6, 8, or 10, wherein at least about 150-200 residues are truncated from its C-terminus.

13. The composition of claim 7, wherein the YRS polypeptide comprises the amino acid sequence of SEQ ID NO: 1, 2, 3, 6, 8, or 10, wherein at least about 200-250 amino acid residues are truncated from its C-terminus.

14. The composition of claim 7, wherein the YRS polypeptide is truncated at its N-terminus.

15. The composition of claim 7, wherein the YRS polypeptide comprises the amino acid sequence of SEQ ID NO: 1, 2, 3, 6, 8, 10, 12, or 14, wherein at least about 1-50 amino acid residues are truncated from its N-terminus.

16. The composition of claim 7, wherein the YRS polypeptide comprises the amino acid sequence of SEQ ID NO: 1, 2, 3, 6, 8, 10, 12, or 14, wherein at least about 50-100 amino acid residues are truncated from its N-terminus.

17. The composition of claim 7, wherein the YRS polypeptide comprises the amino acid sequence of SEQ ID NO: 1, 2, 3, 6, 8, 10, 12, or 14, wherein at least about 100-150 amino acid residues are truncated from its N-terminus.

18. The composition of claim 7, wherein the YRS polypeptide comprises the amino acid sequence of SEQ ID NO: 1, 2, 3, 6, 8, or 10, wherein at least about 150-200 residues are truncated from its N-terminus.

19. The composition of claim 7, wherein the YRS polypeptide comprises the amino acid sequence of SEQ ID NO: 1, 2, 3, 6, 8, or 10, wherein at least about 200-250 amino acid residues are truncated from its N-terminus.

20. The composition of claim 7, wherein the YRS polypeptide comprises an amino acid sequence at least 80%, 90%, 95%, 98%, or 100% identical to the amino acid sequence set forth in SEQ ID NO:2, wherein the alanine at position 341 is not substituted with a tyrosine.

21. The composition of claim 7, wherein the YRS polypeptide comprises an amino acid sequence at least 80%, 90%, 95%, 98%, or 100% identical to the amino acid sequence set forth in SEQ ID NO: 1, 2, 3, 6, 8, 10, 12, or 14.

22. The composition of claim 2, wherein the aminoacyl-tRNA synthetase is a GlyRS polypeptide.

23. The composition of claim 22, wherein the GlyRS polypeptide is a fragment of the full length human glycyl-tRNA synthetase sequence set forth in SEQ ID NO:16.

24. The composition of claim 23, wherein the fragment comprises amino acid residues 367-438 of SEQ ID NO:16, or an active variant thereof

25. The composition of claim 22, wherein the GlyRS polypeptide comprises an amino acid sequence at least 80%, 90%, 95%, 98%, or 100% identical to the amino acid sequence set forth in SEQ ID NO:16.

26. The composition of claim 22, wherein the GlyRS polypeptide comprises amino acid. residues 57-685, 214-685, 239-685, 311-685, 439-685, 511-658, 214-438, 367-438, 214-420, 214-338, 85-127 1-213, 1-61, 85-214, 333-685, 128-685, 265-685, 483-685 or 25-56 of SEQ ID NO:16, or an active fragment thereof.

27. The composition of claim 2, wherein the aminoacyl-tRNA synthetase is a QRS polypeptide.

28. The composition of claim 27, wherein the QRS polypeptide comprises an amino acid sequence at least 80%, 90%, 95%, 98%, or 100% identical to the amino acid sequence set forth in SEQ ID NO:25.

29. The composition of claim 27, wherein the QRS polypeptide is truncated at its C-terminus.

30. The composition of claim 27, wherein the QRS polypeptide comprises the amino acid sequence of SEQ ID NO:25, wherein at least about 1-50 amino acid residues are truncated from its C-terminus.

31. The composition of claim 27, wherein the QRS polypeptide comprises the amino acid sequence of SEQ ID NO:25, wherein at least about 50-100 amino acid residues are truncated from its C-terminus.

32. The composition of claim 27, wherein the QRS polypeptide comprises the amino acid sequence of SEQ ID NO:25, wherein at least about 100-150 amino acid residues are truncated from its C-terminus.

33. The composition of claim 27, wherein the QRS polypeptide comprises the amino acid sequence of SEQ ID NO:25, wherein at least about 150-200 residues are truncated from its C-terminus.

34. The composition of claim 27, wherein the QRS polypeptide comprises the amino acid sequence of SEQ ID NO:25, wherein at least about 200-250 amino acid residues are truncated from its C-terminus.

35. The composition of claim 27, wherein the QRS polypeptide comprises the amino acid sequence of SEQ ID NO:25, wherein at least about 250-350 amino acid residues are truncated from its C-terminus.

36. The composition of claim 27, wherein the QRS polypeptide comprises the amino acid sequence of SEQ ID NO:25, wherein at least about 350-450 amino acid residues are truncated from its C-terminus.

37. The composition of claim 27, wherein the QRS polypeptide comprises the amino acid sequence of SEQ ID NO:25, wherein at least about 450-500 amino acid residues are truncated from its C-terminus.

38. The composition of claim 27, wherein the QRS polypeptide comprises the amino acid sequence of SEQ ID NO:25, wherein at least about 500-550 amino acid residues are truncated from its C-terminus.

39. The composition of claim 27, wherein the QRS polypeptide comprises or consists of amino acid residues 1-183, 1-220, 1-249, or 1-200 of SEQ ID NO:25, or any one or more of SEQ ID NOS:36-108.

40. The composition of claim 2, wherein the aminoacyl-tRNA synthetase is a HisRS polypeptide.

41. The composition of claim 40, comprising a HisRS splice variant polypeptide.

42. The composition of claim 40 or 41, wherein the HisRS polypeptide comprises at least the WHEP domain of HisRS.

43. The composition of claim 40 or 41, wherein the HisRS polypeptide comprises at least the anticodon binding domain of HisRS.

44. The composition of claim 40 or 41, wherein the HisRS polypeptide lacks a functional aminoacylation domain.

45. The composition of claim 40 or 41, wherein the HisRS polypeptide comprises at least the WHEP domain of HisRS and the anticodon binding domain of HisRS but lacks a functional aminoacylation domain.

46. The composition of claim 40, wherein the HisRS polypeptide comprises the sequence set forth in SEQ ID NO:28, 30, or 32.

47. The composition of claim 40, wherein the HisRS polypeptide comprises an amino acid sequence at least 80%, 90%, 95%, 98%, or 100% identical to the amino acid sequence set forth in SEQ ID NO:28, 30, or 32.

48. The composition of claim 40 or 41, wherein the HisRS polypeptide comprises at least 20 contiguous amino acid residues of the sequence set forth in SEQ ID NO:28, 30, or 32.

49. The composition of claim 2, wherein the aminoacyl-tRNA synthetase is a WRS polypeptide.

50. The composition of claim 49, wherein the WRS polypeptide comprises an amino acid sequence at least 80%, 90%, 95%, 98%, or 100% identical to the amino acid sequence set forth in any one or more of SEQ ID NOS:33-35.

51. The composition of claim 49, wherein the WRS polypeptide comprises a biologically active fragment of any one or more of SEQ ID NOS:33-35.

52. A pharmaceutical composition for modulating hematopoiesis in a subject, comprising an aminoacyl-tRNA synthetase (AARS) polypeptide as in any one of claims 1-51 and a pharmaceutically acceptable carrier.

53. A method of modulating hematopoiesis, comprising contacting a cell with an effective concentration of an aminoacyl-tRNA synthetase (AARS) polypeptide having a hematopoiesis-modulating activity, thereby modulating hematopoiesis.

54. The method of claim 53, wherein the cell is a stem cell or a progenitor cell.

55. The method of claim 54, wherein the progenitor cell is a megakaryocyte progenitor cell, an erythrocyte progenitor cell, a lymphocyte progenitor cell, a granulocyte progenitor cell, a monocyte, or an endothelial progenitor cell.

56. The method of claim 53, comprising stimulating the production of at least one of megakaryocyte progenitor cells, erythrocyte progenitor cells, lymphocyte progenitor cells, granulocyte progenitor cells, or monocytes.

57. The method of claim 56, comprising stimulating the production of at least one of megakaryocytes, platelets, erythrocytes, lymphocytes, granulocytes, or macrophages.

58. The method of claim 53, comprising reducing the production of at least one of megakaryocyte progenitor cells, erythrocyte progenitor cells, lymphocyte progenitor cells, granulocyte progenitor cells, or monocytes.

59. The method of claim 58, comprising reducing the production of at least one of megakaryocytes, platelets, erythrocytes, lymphocytes, granulocytes, or macrophages.

60. The method of claim 53, comprising contacting the cell in vitro or ex vivo.

61. The method of claim 60, comprising administering the contacted cells to a subject.

62. The method of claim 53, comprising contacting the cell in a subject by directly administering the AARS polypeptide to the subject.

63. The method of claim 61 or 62, wherein the subject is about to undergo, is undergoing, or has undergone a transplant therapy.

64. The method of claim 63, wherein the transplant therapy is a bone marrow transplant, a cord blood transplant, a hematopoietic stem cell transplant, an autologous peripheral blood cell progenitor transplant, or a liver transplant.

65. The method of claim 61 or 62, wherein the subject is about to undergo, is undergoing, or has undergone chemotherapy or radiotherapy.

66. The method of claim 65, wherein chemotherapy comprises treatment with an agent selected from chlorambucil, cyclophosphamide, lomustine (CCNU), melphalan, procarbazine, thiotepa, carmustine (BCNU), busulfan, daunorubicin, doxorubicin, idarubicin, epirubicin, mitoxantrone, bleomycin, cisplatin, carboplatin, oxaliplatin, camptothecins, irinotecan, topotecan, amsacrine, etoposide; etoposide phosphate, teniposide, vincristine, vinblastine, vinorelbine, vindesine, paclitaxel, mechlorethamine, ifosfamide, nitrosurea, dactinomycin, plicomycin, mitomycin, tamoxifen, raloxifene, estrogen receptor binding agents, gemcitabien, navelbine, farnesyl-protein transferase inhibitors, transplatinum, 5-fluorouracil, methotrexate, and temazolomide.

67. The method of claim 65, wherein chemotherapy comprises high-dose chemotherapy.

68. The method of claim 67, wherein high-dose chemotherapy further comprises a transplant therapy.

69. The method of claim 68, wherein the transplant therapy is a bone marrow transplant, a cord blood transplant, a hematopoietic stem cell transplant, or an autologous peripheral blood cell progenitor transplant.

70. The method of claim 61 or 62, wherein the subject has a condition that is associated with increased red blood cell count.

71. The method of claim 70, wherein the conditions is living at a high altitude, smoking, congenital heart disease, failure of the right side of the heart, pulmonary fibrosis, polycythemia vera, dehydration, kidney disease, kidney, cancer, exposure to carbon monoxide, anabolic steroid use, COPD, or erythropoietin (EPO) doping.

72. The method of claim 61 or 62, wherein the subject has a condition that is associated with decreased red blood count.

73. The method of claim 61 or 62, wherein the subject has a condition that is associated with increased lymphocyte count.

74. The method of claim 61 or 62, wherein the subject has a condition that is associated with decreased lymphocyte count.

75. The method of claim 61 or 62, wherein the subject has a condition that is associated with increased granulocyte count.

76. The method of claim 61 or 62, wherein the subject has a condition that is associated with decreased granulocyte count.

77. The method of claim 61 or 62, wherein the subject has a condition that is associated with increased platelet count.

78. The method of claim 61 or 62, wherein the subject has a condition that is associated with decreased platelet count.

79. A method of modulating hematopoiesis in a subject, comprising administering to the subject an effective concentration of an aminoacyl-tRNA synthetase (AARS) polypeptide, thereby modulating hematopoiesis in the subject.

80. The method of claim 79, comprising increasing the hematopoiesis-stimulatory activity of at least one of an osteoblast cell, a vascular cell, or a neutrophil.

81. The method of claim 79, comprising reducing the hematopoiesis-stimulatory activity of at least one of an osteoblast cell, a vascular cell, or a neutrophil.

82. The method of claim 79, comprising increasing the hematopoiesis-inhibitory activity of at least one of an osteoblast cell or a vascular cell.

83. The method of claim 79, comprising reducing the hematopoiesis-inhibitory activity of at least one of an osteoblast cell or a vascular cell.

85. The method of claim 79, comprising increasing the mobilization of hematopoietic cells from the bone marrow to the periphery of the subject.

86. The method of claim 85, wherein the subject is a peripheral blood cell donor.

87. The method of claim 79, wherein the subject is about to undergo, is undergoing, or has undergone a transplant therapy.

88. The method of claim 79, wherein the subject is about to undergo, is undergoing, or has undergone chemotherapy or radiotherapy.

89. The method of claim 79, wherein the subject has a condition that is associated with at least one of increased red blood cell, increased lymphocyte count, increased granulocyte count, or increased platelet count.

90. The method of claim 79, wherein the subject has a condition that is associated with at least one of decreased red blood cell count, decreased lymphocyte count, decreased granulocyte count, or decreased platelet count.

91. The method of claim 79, comprising co-administering to the subject one or more thrombopoiesis-stimulatory agents selected from thrombopoietin (TPO), a TPO agonist, a TPO mimetic, an mpl-signaling agonist, a cytokine, a chemokine, a chemokine receptor ligand, and an adhesion molecule.

92. The method of claim 91, wherein the co-administration of the agent achieves a synergistic effect.

93. The method of claim 92, wherein the agent is TPO, a TPO agonist, a TPO mimetic, or an mpl-signaling agonist, and the synergistic effect comprises increased thrombopoiesis.

94. The method of claim 91, wherein the agent is eltrombopag or romiplostim.

95. The method of claim 91, wherein the AARS polypeptide is a thrombopoietic YRS polyeptide comprising the sequence set forth in SEQ ID NO:2 (Y341A).

96. A composition, comprising a thrombopoietic AARS polypeptide and a thrombopoiesis-stimulatory agent selected from thrombopoietin (TPO), a TPO agonist, a TPO mimetic, an mpl-signaling agonist, a cytokine, a chemokine, a chemokine receptor ligand, and an adhesion molecule.

97. The composition of claim 96, wherein the agent is TPO, a TPO agonist, a TPO mimetic, or an mpl-signaling agonist.

98. The composition of claim 96, wherein the agent is eltrombopag or romiplostim.

99. The composition of claim 96, wherein the AARS polypeptide is a YRS polyeptide comprising the sequence set forth in SEQ ID NO:2 (Y341A).

100. An isolated polynucleotide encoding a polypeptide according to any one of claims 1-51.

101. A vector comprising the polynucleotide of claim 100.

102. A host cell comprising the vector of claim 101.

103. A fusion polypeptide comprising a polypeptide of any one of claims 1-51 and a heterologous fusion partner.

104. A method of modulating cell division of one or more platelets, comprising contacting the platelet(s) with an aminoacyl-tRNA synthetase (AARS) polypeptide having a hematopoiesis-modulating activity, thereby modulating cell division of the platelet(s).

105. The method of claim 104, comprising increasing cell division of the platelet(s).

106. The method of claim 105, comprising reducing cell division of the platelet(s).

107. An antibody that exhibits binding specificity for an isolated YRS polypeptide of SEQ ID NO:2 or 3, a cellular binding partner of the YRS polypeptide, or both.

108. The antibody of claim 107, wherein affinity of the antibody for the YRS polypeptide of SEQ ID NO:2 or 3 is at least about 10× stronger than its affinity for the YRS polypeptide of SEQ ID NO:1.

109. A binding agent that exhibits binding specificity for an isolated YRS polypeptide of SEQ ID NO:2 or 3, a cellular binding partner of the YRS polypeptide, or both.

110. The binding agent of claim 109, wherein affinity of the binding agent for the YRS polypeptide of SEQ ID NO:2 or 3 is at least about 10× stronger than its affinity for the YRS polypeptide of SEQ ID NO:1.

111. The binding agent of claim 109, selected from a peptide, a soluble receptor, an adnectin, a small molecule, and an aptamer.

112. The antibody or binding agent of any one of claims 107-111, which antagonizes a hematopoiesis-modulating activity of the YRS polypeptide of SEQ ID NO:2 or 3.

113. The antibody or binding agent of any one of claims 107-111, which agonizes a hematopoiesis-modulating activity of the YRS polypeptide of SEQ ID NO:2 or 3.

114. The antibody or binding agent of claim 112 or 113, wherein the hematopoiesis-modulating is a thrombopoietic activity.

115. A method of reducing platelet count in a subject, comprising administering to the subject an antibody or binding agent according to claim 114, thereby reducing platelet count in the subject.

116. The method of claim 115, wherein the subject has thrombocythemia or thrombocytosis.

117. A method of reducing cell division of one or more platelets, comprising contacting the platelet(s) with an antibody or binding agent according to claim 114, thereby reducing cell division of the platelet(s).

118. A method of reducing platelet count in a subject, comprising administering to the subject an AARS polypeptide, wherein the AARS polypeptide reduces thrombopoiesis or megakaryopoiesis, thereby reducing platelet count in the subject.

119. The method of claim 118, wherein the AARS polypeptide is a WRS polypeptide or a YRS polypeptide.

120. The method of claim 119, wherein the YRS polypeptide is a variant of SEQ ID NO:2 which reduces thrombopoiesis or megakaryopoiesis.

121. The method of claim 118, wherein the subject has thrombocythemia or thrombocytosis.


SEQUENCE LISTING (108 sequences)

SEQ ID NO: 1 - protein, 528 aa, Homo sapiens
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 2 - protein, 528 aa, Homo sapiens
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAAPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 3 - protein, 364 aa, Homo sapiens
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVI

SEQ ID NO: 4 - DNA, 1683 bp, Homo sapiens
atgggggacgctcccagccctgaagagaaactgcaccttatcacccggaacctgcaggaggttctgggggaagagaagctgaaggagatactgaaggagcgggaacttaaaatttactggggaacggcaaccacgggcaaaccacatgtggcttactttgtgcccatgtcaaagattgcagacttcttaaaggcagggtgtgaggtaacaattctgtttgcggacctccacgcatacctggataacatgaaagccccatgggaacttctagaactccgagtcagttactatgagaatgtgatcaaagcaatgctggagagcattggtgtgcccttggagaagctcaagttcatcaaaggcactgattaccagctcagcaaagagtacacactagatgtgtacagactctcctccgtggtcacacagcacgattccaagaaggctggagctgaggtggtaaagcaggtggagcaccctttgctgagtggcctcttataccccggactgcaggctttggatgaagagtatttaaaagtagatgcccaatttggaggcattgatcagagaaagattttcacctttgcagagaagtacctccctgcacttggctattcaaaacgggtccatctgatgaatcctatggttccaggattaacaggcagcaaaatgagctcttcagaagaggagtccaagattgatctccttgatcggaaggaggatgtgaagaaaaaactgaagaaggccttctgtgagccaggaaatgtggagaacaatggggttctgtccttcatcaagcatgtcctttttccccttaagtccgagtttgtgatcctacgagatgagaaatggggtggaaacaaaacctacacagcttacgtggacctggaaaaggactttgctgctgaggttgtacatcctggagacctgaagaattctgttgaagtcgcactgaacaagttgctggatccaatccgggaaaagtttaatacccctgccctgaaaaaactggccagcgctgcctacccagatccctcaaagcagaagccaatggccaaaggccctgccaagaattcagaaccagaggaggtcatcccatcccggctggatatccgtgtggggaaaatcatcactgtggagaagcacccagatgcagacagcctgtatgtagagaagattgacgtgggggaagctgaaccacggactgtggtgagcggcctggtacagttcgtgcccaaggaggaactgcaggacaggctggtagtggtgctgtgcaacctgaaaccccagaagatgagaggagtcgagtcccaaggcatgcttctgtgtgcttctatagaagggataaaccgccaggttgaacctctggaccctccggcaggctctgctcctggtgagcacgtgtttgtgaagggctatgaaaagggccaaccagatgaggagctcaagcccaagaagaaagtcttcgagaagttgcaggctgacttcaaaatttctgaggagtgcatcgcacagtggaagcaaaccaacttcatgaccaagctgggctccatttcctgtaaatcgctgaaaggggggaacattagctagccagcccagcatcttccccccttcttccaccactgagtcatctgctgtctcttcagtctgctccatccatcacccatttacccatctctcaggaca

SEQ ID NO: 5 - protein, 8 aa, Artificial Sequence
  C-terminal tag
LEHHHHHH

SEQ ID NO: 6 - protein, 348 aa, Homo sapiens
  VARIANT 1, 2, 3, 4, 5, 6, 7, 8, 9: Xaa = Any Amino Acid
XXXXXXXXXKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 7 - DNA, 2178 bp, Homo sapiens
ttcagaaagtggtggagggaagacttcctttttcccagagacagaaggttatgcacccagtggcctgggaccattgttctgggctttttttcccttcgacatggatttgcttctcactgtgtaccccaaccaccaaaaccaccctgagatcaatgctggtgctcctgcatcagatggcttagagatccttccacctcttaacacaagcatctaggtccactttactcaaatctggcctcagttgagagcagagtataccatcagagcccattctcctgtctgctgtctgggacgtggaaagaaagttagctctagggggtctttccaggggcctctgtaaggactggatgctcctttccggaatccaagagttcaccaggctgcttctctaatggacgatgatcctcttcctcctgacgtctctccctggcagcacccagatgcagacagcctgtatgtagagaagattgacgtgggggaagctgaaccacggactgtggtgagcggcctggtacagttcgtgcccaaggaggaactgcaggacaggctggtagtggtgctgtgcaacctgaaaccccagaagatgagaggagtcgagtcccaaggcatgcttctgtgtgcttctatgtgagtgaggacttggagtggggcacaggacctggggaggccaggaagagtagggaatcagcccatatgatgtccttccacacaccaggtggaagctctgagaacacgtgcctcttccttgctgatgccaaaagttgatgcatgaaggacttatcgtacaagtactgttaatgaagcattttacctacagttaattttgttaaaatagaaatggagggctcaaaccagtacatacccaagtcttactactagtaaggagtggagcagggattcaaatcccagttttgatgtctataaagtcctcgctacgttattttatacttcctcccctagaaacacagattttggtatcttgacacacaattttggtatagcctgggttaatgtaaccctggtgatatgcagggatgtagcaagataagaggacctcctggggctctggtactgaggatgccctaaatcccatcagggcccctgtgtaaaggcccggattgctttggcctccacagtcactggaacccatccatagcctcactcttctcttgtcctgtgtcttcccagagaagggataaaccgccaggttgaacctctggaccctccggcaggctctgctcctggtgagcacgtgtttgtgaagggctatgaaaagggccaaccagatgaggagctcaagcccaagaggaaagtcttcgagaagttgcaggctgacttcaaaatttctgaggagtgcatcgcacagtggaagcaaaccaacttcatgaccaagctgggctccatttcctgtaaatcgctgaaaggggggaacattagctagccagcccagcatcttccccccttcttccaccactgagtcatctgctgtctcttcagtctgctccacccatcacccatttacccatctctcaggacacggaagcagcgggtttggactctttattcggtgcagaactcggcaaggggcagcttaccctccccagaacccaggatcatcctgtctggctgcagtgagagaccaacccctaacaagggctgggccacagcagggagtccagccctaccttcttcccttggcagctggagaaatctggtttcaatataactcatttaaaaatttatgccacagtccttataattggaaaaatactggtgcccaggttttcttggagttatccaagcagctgcgcccctagctgggatctggtacctggactaggctaattacagcttctccccaacaggaaactgtgggatttgaaaaggaaagggaagggaaaacagagaacctagtggtctaccaagtggttggcaactttcccaatgtctgcttactctgaggcttggcactgggggccagggcctgccccagggctcctggaatttcccttgatccagctaggctgggacactccctaaatcagctgcgtgttgttagcatcaggcagaatgaatggcagagagtgattctgtcttcatagagggtggggtacttctccataaggcatctcagtcaaatccccatcactgtcataaattcaaataaaatgtctgaac

SEQ ID NO: 8 - protein, 388 aa, Homo sapiens
  VARIANT 354, 355, 356, 357, 358, 359, 360, 361, 362, 363, 364,365, 366, 367, 368, 369, 370, 371, 372, 373, 374, 375, 376, 377,378, 379, 380, 381, 382, 383, 384, 385, 386, 387, 388: Xaa = Any Amino Acid
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGXXXXXXXXXXXXXXXXXXXXXXXXXXXXXXXXXXX

SEQ ID NO: 9 - DNA, 1167 bp, Homo sapiens
atgggggacgctcccagccctgaagagaaactgcaccttatcacccggaacctgcaggaggttctgggggaagagaagctgaaggagatactgaaggagcgggaacttaaaatttactggggaacggcaaccacgggcaaaccacatgtggcttactttgtgcccatgtcaaagattgcagacttcttaaaggcagggtgtgaggtaacaattctgtttgcggacctccacgcatacctggataacatgaaagccccatgggaacttctagaactccgagtcagttactatgagaatgtgatcaaagcaatgctggagagcattggtgtgcccttggagaagctcaagttcatcaaaggcactgattaccagctcagcaaagagtacacactagatgtgtacagactctcctccgtggtcacacagcacgattccaagaaggctggagctgaggtggtaaagcaggtggagcaccctttgctgagtggcctcttataccccggactgcaggctttggatgaagagtatttaaaagtagatgcccaatttggaggcattgatcagagaaagattttcacctttgcagagaagtacctccctgcacttggctattcaaaacgggtccatctgatgaatcctatggttccaggattaacaggcagcaaaatgagctcttcagaagaggagtccaagattgatctccttgatcggaaggaggatgtgaagaaaaaactgaagaaggccttctgtgagccaggaaatgtggagaacaatggggttctgtccttcatcaagcatgtcctttttccccttaagtccgagtttgtgatcctacgagatgagaaatggggtggaaacaaaacctacacagcttacgtggacctggaaaaggactttgctgctgaggttgtacatcctggagacctgaagaattctgttgaagtcgcactgaacaagttgctggatccaatccgggaaaagtttaatacccctgccctgaaaaaactggccagcgctgcctacccagatccctcaaagcagaagccaatggccaaaggcctgccaagaattcagaaccagaggaggtcatcccatcccggctggatatccgtgtggggaaaatcatcactgtggagaagcacccagatgcagacagcctgtatgtag

SEQ ID NO: 10 - protein, 318 aa, Homo sapiens
MNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 11 - DNA, 1736 bp, Homo sapiens
gaaagattttcacctttgcagagaagtacctccctgcacttggctattcaaaacgggtccatctgatgaatcctatggttccaggattaacaggcagcaaaatgagctcttcagaagaggagtccaagattgatctccttgatcggaaggaggatgtgaagaaaaaactgaagaaggccttctgtgagccaggaaatgtggagaacaatggggttctgtccttcatcaagcatgtcctttttccccttaagtccgagtttgtgatcctacgagatgagaaatggggtggaaacaaaacctacacagcttacgtggacctggaaaaggactttgctgctgaggttgtacatcctggagacctgaagaattctgttgaagtcgcactgaacaagttgctggatccaatccgggaaaagtttaatacccctgccctgaaaaaactggccagcgctgcctacccagatccctcaaagcagaagccaatggccaaaggccctgccaagaattcagaaccagaggaggtcatcccatcccggctggatatccgtgtggggaaaatcatcactgtggagaagcacccagatgcagacagcctgtatgtagagaagattgacgtgggggaagctgaaccacggactgtggtgagcggcctggtacagttcgtgcccaaggaggaactgcaggacaggctggtagtggtgctgtgcaacctgaaaccccagaagatgagaggagtcgagtcccaaggcatgcttctgtgtgcttctatagaagggataaaccgccaggttgaacctctggaccctccggcaggctctgctcctggtgagcacgtgtttgtgaagggctatgaaaagggccaaccagatgaggagctcaagcccaagaagaaagtcttcgagaagttgcaggctgacttcaaaatttctgaggagtgcatcgcacagtggaagcaaaccaacttcatgaccaagctgggctccatttcctgtaaatcgctgaaaggggggaacattagctagccagcccagcatcttccccccttcttccaccactgagtcatctgctgtctcttcagtctgctccatccatcacccatttacccatctctcaggacacggaagcagcgggtttggactctttattcggtgcagaactcggcaaggggcagcttaccctccccagaacccaggatcatcctgtctggctgcagtgagagaccaacccctaacaagggctgggccacagcagggagtccagccctaccttcttcccttggcagctggagaaatctggtttcaatataactcatttaaaaatttatgccacagtccttataattggaaaaatactggtgcccaggttttcttggagttatccaagcagctgcgcccctagctgggatctggtacctggactaggctaattacagcttctccccaacaggaaactgtgggatttgaaaaggaaagggaagggaaaacagagaacctagtggtctaccaagtggttggcaactttcccaatgtctgcttactctgaggcttggcactgggggccagggcctgccccagggctcctggaatttcccttgatccagctaggctgggacactccctaaatcagctgcgtgttgttagcatcaggcagaatgaatggcagagagtgattctgtcttcatagagggtggggtacttctccataaggcatctcagtcaaatccccatcactgtcat

SEQ ID NO: 12 - protein, 179 aa, Homo sapiens [sequence omitted]

SEQ ID NO: 13 - DNA, 1167 bp, Homo sapiens; identical to SEQ ID NO: 9

SEQ ID NO: 14 - protein, 188 aa, Homo sapiens
  VARIANT 1, 2, 3, 4, 5, 6, 7, 8: Xaa = Any Amino Acid
XXXXXXXXPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 15 - DNA, 2262 bp, Homo sapiens
gccagacacagtggctcacacctgtaatcttaacactttggaaggctgaggcaggcggatcacttgagcccaaaagttagagaccaaaacccagtctctacccaaaaaaaaaaaaaaaaaaaaaattagccaggcatagtagcacatgcctgtagtcccagctacttgggaggctgaggtgagaggatcacctgagcatggggaagttgagactgcagtgagccatgatcgcaccactgcactccagcctgggcaacagagtgagactctatgtctcaaaaaaagaaaaatgatagaaattagattagacctattatacccaaccggtatatagggtatcgatagtttcttacacagctgttgggcagagcctgcagagcttagagaagcttatctttagattctcccagtttccttctatgtgcatgggcctggctcttagttggccatccacttgtgcgtaatgctaagatattggcattgatagctttgtgcgacccttccagaaaaaaactcagtaactcagtaaaatttttttttttttttctaaaagagacagagtctggctctgttgcccagcctggtcttgaagtcctgggcttaagcaatcctcccgtctcagcctcccaaagtgctagaattacaggtgtgagctaccacacctggccaagactcagtaaattctatgtggaatgcatgaatggaaatacctaaaggaggcaaagctactactgctccctccccgctagtctaataattgagggagagaacagatgaaaatcaggtatgtcatgtctgaaaggttgccaacccagtattaaagaagttacaactcagtgtttagactctggggattctacactaaatcttacctaatctcagtgtcttaacgtggtgggatcagcagctgacctgccacagggaagaattctacctcatggggttcttctcattcccagagccaatggccaaaggccctgccaagaattcagaaccagaggaggtcatcccatcccggctggatatccgtgtggggaaaatcatcactgtggagaagcacccagatgcagacagcctgtatgtagagaagattgacgtgggggaagctgaaccacggactgtggtgagcggcctggtacagttcgtgcccaaggaggaactgcaggacaggctggtagtggtgctgtgcaacctgaaaccccagaagatgagaggagtcgagtcccaaggcatgcttctgtgtgcttctatagaagggataaaccgccaggttgaacctctggaccctccggcaggctctgctcctggtgagcacgtgtttgtgaagggctatgaaaagggccaaccagatgaggagctcaagcccaagaagaaagtcttcgagaagttgcaggctgacttcaaaatttctgaggagtgcatcgcacagtggaagcaaaccaacttcatgaccaagctgggctccatttcctgtaaatcgctgaaaggggggaacattagctagccagcccagcatcttccccccttcttccaccactgagtcatctgctgtctcttcagtctgctccatccatcacccatttacccatctctcaggacacggaagcagcgggtttggactctttattcggtgcagaactcggcaaggggcagcttaccctccccagaacccaggatcatcctgtctggctgcagtgagagaccaacccctaacaagggctgggccacagcagggagtccagccctaccttcttcccttggcagctggagaaatctggtttcaatataactcatttaaaaatttatgccacagtccttataattggaaaaatactggtgcccaggttttcttggagttatccaagcagctgcgcccctagctgggatctggtacctggactaggctaattacagcttctccccaacaggaaactgtgggatttgaaaaggaaagggaagggaaaacagagaacctagtggtctaccaagtggttggcaactttcccaatgtctgcttactctgaggcttggcactgggggccagggcctgccccagggctcctggaatttcccttgatccagctaggctgggacactccctaaatcagctgcgtgttgttagcatcaggcagaatgaatggcagagagtgattctgtcttcatagagggtggggtacttctccataaggcatctcagtcaaatccccatcactgtcataaattcaaataaaatgtctgaacaagggaaaaaaaaaaaaaaaa

SEQ ID NO: 16 - protein, 685 aa, Homo sapiens
MDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 17 - DNA, 2058 bp, Homo sapiens
atggacggcgcgggggctgaggaggtgctggcacctctgaggctagcagtgcgccagcagggagatcttgtgcgaaaactcaaagaagataaagcaccccaagtagacgtagacaaagcagtggctgagctcaaagcccgcaagagggttctggaagcaaaggagctggcgttacagcccaaagatgatattgtagaccgagcaaaaatggaagataccctgaagaggaggtttttctatgatcaagcttttgctatttatggaggtgttagtggtctgtatgactttgggccagttggctgtgctttgaagaacaatattattcagacctggaggcagcactttatccaagaggaacagatcctggagatcgattgcaccatgctcacccctgagccagttttaaagacctctggccatgtagacaaatttgctgacttcatggtgaaagacgtaaaaaatggagaatgttttcgtgctgaccatctattaaaagctcatttacagaaattgatgtctgataagaagtgttctgtcgaaaagaaatcagaaatggaaagtgttttggcccagcttgataactatggacagcaagaacttgcggatctttttgtgaactataatgtaaaatctcccattactggaaatgatctatcccctccagtgtcttttaacttaatgttcaagactttcattgggcctggaggaaacatgcctgggtacttgagaccagaaactgcacaggggattttcttgaatttcaaacgacttttggagttcaaccaaggaaagttgccttttgctgctgcccagattggaaattcttttagaaatgagatctcccctcgatctggactgatcagagtcagagaattcacaatggcagaaattgagcactttgtagatcccagtgagaaagaccaccccaagttccagaatgtggcagaccttcacctttatttgtattcagcaaaagcccaggtcagcggacagtccgctcggaaaatgcgcctgggagatgctgttgaacagggtgtgattaataacacagtattaggctatttcattggccgcatctacctctacctcacgaaggttggaatatctccagataaactccgcttccggcagcacatggagaatgagatggcccattatgcctgtgactgttgggatgcagaatccaaaacatcctacggttggattgagattgttggatgtgctgatcgttcctgttatgacctctcctgtcatgcacgagccaccaaagtcccacttgtagctgagaaacctctgaaagaacccaaaacagtcaatgttgttcagtttgaacccagtaagggagcaattggtaaggcatataagaaggatgcaaaactggtgatggagtatcttgccatttgtgatgagtgctacattacagaaatggagatgctgctgaatgagaaaggggaattcacaattgaaactgaagggaaaacatttcagttaacaaaagacatgatcaatgtgaagagattccagaaaacactatatgtggaagaagttgttccgaatgtaattgaaccttccttcggcctgggtaggatcatgtatacggtatttgaacatacattccatgtacgagaaggagatgaacagagaacattcttcagtttccctgctgtagttgctccattcaaatgttccgtcctcccactgagccaaaaccaggagttcatgccatttgtcaaggaattatcggaagccctgaccaggcatggagtatctcacaaagtagacgattcctctgggtcaatcggaaggcgctatgccaggactgatgagattggcgtggcttttggtgtcaccattgactttgacacagtgaacaagaccccccacactgcaactctgagggaccgtgactcaatgcggcagataagagcagagatctctgagctgcccagcatagtccaagacctagccaatggcaacatcacatgggctgatgtggaggccaggtatcctctgtttgaagggcaagagactggtaaaaaagagacaatcgaggaatga

SEQ ID NO: 18 - protein, 17 aa, Homo sapiens
PGYLRPETAQGIFLNFK

SEQ ID NO: 19 - protein, 16 aa, Homo sapiens
TGNDLSPPVSFNLMFK

SEQ ID NO: 20 - protein, 9 aa, Homo sapiens
NVVQFEPSK

SEQ ID NO: 21 - protein, 19 aa, Homo sapiens
LYVEEVVPNVIEPSFGLGR

SEQ ID NO: 22 - protein, 20 aa, Homo sapiens
AIYGGVSGLYDFGPVGCALK

SEQ ID NO: 23 - protein, 15 aa, Homo sapiens
QHFIQEEQILEIDCT

SEQ ID NO: 24 - skipped

SEQ ID NO: 25 - protein, 775 aa, Homo sapiens
MAALDSLSLFTSLGLSEQKARETLKNSALSAQLREAATQAQQTLGSTIDKATGILLYGLASRLRDTRRLSFLVSYIASKKIHTEPQLSAALEYVRSHPLDPIDTVDFERECGVGVIVTPEQIEEAVEAAINRHRPQLLVERYHFNMGLLMGEARAVLKWADGKMIKNEVDMQVLHLLGPKLEADLEKKFKVAKARLEETDRRTAKDVVENGETADQTLSLMEQLRGEALKFHKPGENYKTPGYVVTPHTMNLLKQHLEITGGQVRTRFPPEPNGILHIGHAKAINFNFGYAKANNGICFLRFDDTNPEKEEAKFFTAICDMVAWLGYTPYKVTYASDYFDQLYAWAVELIRRGLAYVCHQRGEELKGHNTLPSPWRDRPMEESLLLFEAMRKGKFSEGEATLRMKLVMEDGKMDPVAYRVKYTPHHRTGDKWCIYPTYDYTHCLCDSIEHITHSLCTKEFQARRSSYFWLCNALDVYCPVQWEYGRLNLHYAVVSKRKILQLVATGAVRDWDDPRLFTLTALRRRGFPPEAINNFCARVGVTVAQTTMEPHLLEACVRDVLNDTAPRAMAVLESLRVIITNFPAAKSLDIQVPNFPADETKGFHQVPFAPIVFIERTDFKEEPEPGFKRLAWGQPVGLRHTGYVIELQHVVKGPSGCVESLEVTCRRADAGEKPKAFIHWVSQPLMCEVRLYERLFQHKNPEDPTEVPGGFLSDLNLASLHVVDAALVDCSVALAKPFDKFQFERLGYFSVDPDSHQGKLVFNRTVTLKEDPGKV

SEQ ID NO: 26 - protein, 15 aa, Homo sapiens
DVVENGETADQTLSL

SEQ ID NO: 27 - protein, 10 aa, Homo sapiens
TPGYVVTPHT

SEQ ID NO: 28 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 29 - DNA, 183 bp, Homo sapiens
atggcagagcgtgcggcgctggaggagctggtgaaacttcagggagagcgcgtgcgaggcctcaagcagcagaaggccagcgccgagctgatcgaggaggaggtggcgaaactcctgaaactgaaggcacagctgggtcctgatgaaagcaaacagaaatttgtgctcaaaacccccaagtag

SEQ ID NO: 30 - protein, 60 aa, Homo sapiens
MAERAALEELVKLQGERVRGLKQQKASAELIEEEVAKLLKLKAQLGPDESKQKFVLKTPK

SEQ ID NO: 31 - DNA, 516 bp, Homo sapiens
atggcagagcgtgcggcgctggaggagctggtgaaacttcagggagagcgcgtgcgaggcctcaagcagcagaaggccagcgccgagctgatcgaggaggaggtggcgaaactcctgaaactgaaggcacagctgggtcctgatgaaagcaaacagaaatttgtgctcaaaacccccaaggctttggaggagaagatacggaccacggagacacaggtgcttgtggcatctgcacagaagaagctgctagaggaaagactaaagcttgtctcagaactgtgggatgctgggatcaaggctgagctgctgtacaagaagaacccaaagctactgaaccagttacagtactgtgaggaggcaggcatcccactggtggctatcatcggcgagcaggaactcaaggatggggtcatcaagctccgttcagtgacgagcagggaagaggtggatgtccgaagagaagaccttgtggaggaaatcaaaaggagaacaggccagcccctctgcatctgctga

SEQ ID NO: 32 - protein, 171 aa, Homo sapiens
MAERAALEELVKLQGERVRGLKQQKASAELIEEEVAKLLKLKAQLGPDESKQKFVLKTPKALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 33 - protein, 471 aa, Homo sapiens
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 34 - protein, 379 aa, Homo sapiens
MSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 35 - protein, 424 aa, Homo sapiens [sequence omitted]

SEQ ID NO: 36 - protein, 13 aa, Mus musculus
ETLKNEALSTQLR

SEQ ID NO: 37 - protein, 28 aa, Mus musculus
EAATQAHQILGSTIDKATGVLLYDLVSR

SEQ ID NO: 38 - protein, 41 aa, Mus musculus
ETLKNEALSTQLREAATQAHQILGSTIDKATGVLLYDLVSR

SEQ ID NO: 39 - protein, 27 aa, Mus musculus
DFEQECGVGVVVTPEQIEEAVESTINK

SEQ ID NO: 40 - protein, 11 aa, Mus musculus
FNMGLLMGEAR

SEQ ID NO: 41 - protein, 17 aa, Mus musculus
MIKNEVDMQVLHLLGPK

SEQ ID NO: 42 - protein, 14 aa, Mus musculus
NEVDMQVLHLLGPK

SEQ ID NO: 43 - protein, 15 aa, Mus musculus
TPGYVITPYTMDLLK

SEQ ID NO: 44 - protein, 12 aa, Mus musculus
FDDTNPEKEEAK

SEQ ID NO: 45 - protein, 15 aa, Mus musculus
VEELKGHNPLPSPWR

SEQ ID NO: 46 - protein, 15 aa, Mus musculus
DRPKEESLLLFEAMR

SEQ ID NO: 47 - protein, 30 aa, Mus musculus
VEELKGHNPLPSPWRDRPKEESLLLFEAMR

SEQ ID NO: 48 - protein, 14 aa, Mus musculus
LVMEDGKMDPVAYR

SEQ ID NO: 49 - protein, 11 aa, Mus musculus
VYCPVQWEYGR

SEQ ID NO: 50 - protein, 11 aa, Mus musculus
ILQLVAAGAVR

SEQ ID NO: 51 - protein, 28 aa, Mus musculus
DVLNDAAPRAMAVLEPLQVVITNFPAPK

SEQ ID NO: 52 - protein, 38 aa, Mus musculus
GFHQVPFASTVFIERSDFKEESEPGYKRLASGQPVGLR

SEQ ID NO: 53 - protein, 15 aa, Mus musculus
AFIHWVSQPLVCEIR

SEQ ID NO: 54 - protein, 19 aa, Mus musculus
LGYFSVDPDSHQGQIVFNR

SEQ ID NO: 55 - protein, 15 aa, Mus musculus
TPGYVITPYTMDLLK

SEQ ID NO: 56 - protein, 10 aa, Mus musculus
AINFNFGYAK

SEQ ID NO: 57 - protein, 12 aa, Mus musculus
FDDTNPEKEEAK

SEQ ID NO: 58 - protein, 18 aa, Mus musculus
FFTAIYDMVTWLGYTPYK

SEQ ID NO: 59 - protein, 30 aa, Mus musculus
FDDTNPEKEEAKFFTAIYDMVTWLGYTPYK

SEQ ID NO: 60 - protein, 15 aa, Mus musculus
DRPKEESLLLFEAMR

SEQ ID NO: 61 - protein, 11 aa, Mus musculus
VYCPVQWEYGR

SEQ ID NO: 62 - protein, 10 aa, Mus musculus
LNLHYAVVSK

SEQ ID NO: 63 - protein, 21 aa, Mus musculus
VYCPVQWEYGRLNLHYAVVSK

SEQ ID NO: 64 - protein, 11 aa, Mus musculus
ILQLVAAGAVR

SEQ ID NO: 65 - protein, 19 aa, Mus musculus
AMAVLEPLQVVITNFPAPK

SEQ ID NO: 66 - protein, 15 aa, Mus musculus
PLDIRVPNFPADETK

SEQ ID NO: 67 - protein, 34 aa, Mus musculus
AMAVLEPLQVVITNFPAPKPLDIRVPNFPADETK

SEQ ID NO: 68 - protein, 36 aa, Mus musculus
SDFKEESEPGYKRLASGQPVGLRHTGYVIELQNIVR

SEQ ID NO: 69 - protein, 15 aa, Mus musculus
AFIHWVSQPLVCEIR

SEQ ID NO: 70 - protein, 19 aa, Mus musculus
LGYFSVDPDSHQGQIVFNR

SEQ ID NO: 71 - protein, 13 aa, Mus musculus
KATGVLLYDLVSR

SEQ ID NO: 72 - protein, 10 aa, Mus musculus
SFLVSYIANK

SEQ ID NO: 73 - protein, 27 aa, Mus musculus
DFEQECGVGVVVTPEQIEEAVESTINK

SEQ ID NO: 74 - protein, 17 aa, Mus musculus
MIKNEVDMQVLHLLGPK

SEQ ID NO: 75 - protein, 28 aa, Mus musculus
EAATQAHQILGSTIDKATGVLLYDLVSR

SEQ ID NO: 76 - protein, 13 aa, Homo sapiens
ETLKNSALSAQLR

SEQ ID NO: 77 - protein, 28 aa, Homo sapiens
EAATQAQQTLGSTIDKATGILLYGLASR

SEQ ID NO: 78 - protein, 41 aa, Homo sapiens
ETLKNSALSAQLREAATQAQQTLGSTIDKATGILLYGLASR

SEQ ID NO: 79 - protein, 27 aa, Homo sapiens
DFERECGVGVIVTPEQIEEAVEAAINR

SEQ ID NO: 80 - protein, 15 aa, Homo sapiens
TPGYVVTPHTMNLLK

SEQ ID NO: 81 - protein, 15 aa, Homo sapiens
GEELKGHNTLPSPWR

SEQ ID NO: 82 - protein, 15 aa, Homo sapiens
DRPMEESLLLFEAMR

SEQ ID NO: 83 - protein, 30 aa, Homo sapiens
GEELKGHNTLPSPWRDRPMEESLLLFEAMR

SEQ ID NO: 84 - protein, 11 aa, Homo sapiens
ILQLVATGAVR

SEQ ID NO: 85 - protein, 28 aa, Homo sapiens
DVLNDTAPRAMAVLESLRVIITNFPAAK

SEQ ID NO: 86 - protein, 38 aa, Homo sapiens
GFHQVPFAPIVFIERTDFKEEPEPGFKRLAWGQPVGLR

SEQ ID NO: 87 - protein, 15 aa, Homo sapiens
AFIHWVSQPLMCEVR

SEQ ID NO: 88 - protein, 19 aa, Homo sapiens
LGYFSVDPDSHQGKLVFNR

SEQ ID NO: 89 - protein, 15 aa, Homo sapiens
TPGYVVTPHTMNLLK

SEQ ID NO: 90 - protein, 18 aa, Homo sapiens
FFTAICDMVAWLGYTPYK

SEQ ID NO: 91 - protein, 30 aa, Homo sapiens; identical to SEQ ID NO: 59

SEQ ID NO: 92 - protein, 15 aa, Homo sapiens
DRPMEESLLLFEAMR

SEQ ID NO: 93 - protein, 11 aa, Homo sapiens
ILQLVATGAVR

SEQ ID NO: 94 - protein, 19 aa, Homo sapiens
AMAVLESLRVIITNFPAAK

SEQ ID NO: 95 - protein, 15 aa, Homo sapiens
SLDIQVPNFPADETK

SEQ ID NO: 96 - protein, 34 aa, Homo sapiens
AMAVLESLRVIITNFPAAKSLDIQVPNFPADETK

SEQ ID NO: 97 - protein, 36 aa, Homo sapiens
TDFKEEPEPGFKRLAWGQPVGLRHTGYVIELQHVVK

SEQ ID NO: 98 - protein, 15 aa, Homo sapiens
AFIHWVSQPLMCEVR

SEQ ID NO: 99 - protein, 19 aa, Homo sapiens
LGYFSVDPDSHQGKLVFNR

SEQ ID NO: 100 - protein, 13 aa, Homo sapiens
KATGILLYGLASR

SEQ ID NO: 101 - protein, 10 aa, Homo sapiens
SFLVSYIASK

SEQ ID NO: 102 - protein, 27 aa, Homo sapiens
DFERECGVGVIVTPEQIEEAVEAAINR

SEQ ID NO: 103 - protein, 28 aa, Homo sapiens
EAATQAQQTLGSTIDKATGILLYGLASR

SEQ ID NO: 104 - protein, 86 aa, Homo sapiens
MPTVSVKRDLLFQALGRTYTDEEFDELCFEFGLELDEIKDLELLCPRCSLSTDHQHRCVWPIKKAASYQEKTGAIQCTVEGESGTN

SEQ ID NO: 105 - protein, 501 aa, Homo sapiens
MPSASASRKSQEKPREIMDAAEDYAKERYGISSMIQSQEKPDRVLVRVRDLTIQKADEVVWVRARVHTSRAKGKQCFLVLRQQQFNVQALVAVGDHASKQMVKFAANINKESIVDVEGVVRKVNQKIGSCTQQDVELHVQKIYVISLAEPRLPLQLDDAVRPEAEGEEEGRATVNQDTRLDNRVIDLRTSTSQAVFRLQSGICHLFRETLINKGFVEIQTPKIISAASEGGANVFTVSYFKNNAYLAQSPQLYKQMCICADFEKVFSIGPVFRAEDSNTHRHLTEFVGLDIEMAFNYHYHEVMEEIADTMVQIFKGLQERFQTEIQTVNKQFPCEPFKFLEPTLRLEYCEALAMLREAGVEMGDEDDLSTPNEKLLGHLVKEKYDTDFYILDKYPLAVRPFYTMPDPRNPKQSNSYDMFMRGEEILSGAQRIHDPQLLTERALHHGIDLEKIKAYIDSFRFGAPPHAGGGIGLERVTMLFLGLHNVRQTSMFPRDPKRLTP

SEQ ID NO: 106 - protein, 471 aa, Homo sapiens; identical to SEQ ID NO: 33

SEQ ID NO: 107 - protein, 424 aa, Homo sapiens [sequence omitted]

SEQ ID NO: 108 - protein, 401 aa, Homo sapiens
SNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ